SEQUENCE LISTING (4 sequences)

SEQ ID NO: 1 - protein, 532 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 2 - DNA, 6417 bp, Homo sapiens
aagagtcggcggcggtggcggcggccgctgcagagattggaatccgcctgccgggcttggcgaaggagaagggaggaggcaggagcgaggagggaggagggccaagggcgggcaggaaggcttaggctcggcgcgtccgtccgcgcgcggcgaagatcgcacggcccgatcgaggggcgaccgggtcggggccgctgcacgccaagggcgaaggccgattcgggccccacttcgccccggcggctcgccgcgcccacccgctccgcgccgagggctggaggatgcgttccctggggtccggacttatgaaaatatgcatcagtttaatactgtcttggaattcatgagatggaagcataggtcaaagctgtttggagaaaatcagaagtacagttttatctagccacatcttggaggagtcgtaagaaagcagtgggagttgaagtcattgtcaagtgcttgcgatcttttacaagaaaatctcactgaatgatagtcatttaaattggtgaagtagcaagaccaattattaaaggtgacagtacacaggaaacattacaattgaacaatgcctcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctgatggttaaaccatcggaggagaaactctagactgcaagaactgtttttacccatggcatgggtggaattagagtggaataaggatgttaacttggttctcagactctttcttcactacgtgttcacaggctgctaatattaaacctttcagtactcttattaggatacaagctgggaacttctaaacacttcattctttatatatggacagctttattttaaatgtggtttttgatgcctttttttaagtgggtttttatgaactgcatcaagacttcaatcctgattagtgtctccagtcaagctctgggtactgaattgcctgttcataaaacggtgctttctgtgaaagccttaagaagataaatgagcgcagcagagatggagaaatagactttgccttttacctgagactttcagttcgtttgtattctacctttgtaaaacagcctatagatgatgatgtgtttgggatactgcttattttatgatagtttgtcctgtgtccttagtgatgtgtgtgtgtctccatgcacatgcacgccgggattcctctgctgccatttgaattagaagaaaataatttatatgcatgcacaggaagatattggtggccggtggttttgtgctttaaaaatgcaatatctgaccaagattcgccaatctcatacaagccatttactttgcaagtgagatagcttccccaccagctttattttttaacatgaaagctgatgccaaggccaaaagaagtttaaagcatctgtaaatttggactgttttccttcaaccaccattttttttgtggttattatttttgtcacggaaagcatcctctccaaagttggagcttctattgccatgaaccatgcttacaaagaaagcacttcttattgaagtgaattcctgcatttgatagcaatgtaagtgcctataaccatgttctatattctttattctcagtaacttttaaaagggaagttatttatattttgtgtataatgtgctttatttgcaaatcacccactcctttacaaccatactttatatatgtacatacattcatactgtagaaaccagctcatgtgtacctcatatcccatccttaagagaagaaatgttataaagtagaactaaatataaattttcagaattaatgcattcaaagtaatatatcaaatccaggactttgttaacttcaggtaaaaacttcattagggtaatatcatctcaattttttcaaatgaaaggattctctaattagaaatttatatgtcagagctgttataaatttatcaactgtcaaatatgttctggacagctaaatcatttgagatttttggttttttgatttctattccctaacttgtgaagacaatgaaaaatcaggcagaaatatttagtatctagtcagtatctgtagctacactgtataactgttcttcaataaaatggttcatattttatagatgccttgttatctcaagaaatctgatttacataaacttatacttctttaatgctttttaaatatttattctgagcaaacaattcatgagtacatcaagtgagatagttttatttgattataacataaaataaatgtgattatatcacatcatcatcaaaaaggtttaaattaaatgggaggaaatcagcatatgtccacccattaccaaaatttgactatcatttaaggttaaaacttacaaatttgtctgcacatcaaatttcacaaatttgaaaattgccttaaccattttgattaataagtttcatctgccataattaaagtctgaagtgttcatcaagataagtaaatttgcatatggataatacccaataacttgttttttcagaatttttcaccatatgtatactgagaaatacaaatattttaatctgcgttgccgtatgatatgattgcacttagaacacccaatttacttaaatcttggtttacttttgacttgataccataatctttaaaatcatttgtcatctttttttttttttttttgagacggagtctcgctctgtcgcccaggctggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcaatctcctgacctcatgatccacctgcctcggcctcccaaagttcatttgtcatcttaataaaaatataaagacaggcaaagtttattggaaatgttcaaatggtgtgtggaagcaaaaaattacagccagtatatgagaccactattatggttttttaaaattaacttggtctagtaaaagtgatatcaagagttaatcttagaaacttgctcagtaaaaacattttctagtataacatgttctttaaaaagcaaatgctgccgtctttggaatcttaatctaaaaatgtggccgggcgcggtggctcacgcctgtaatcccaacactttgggaggctgaggcgggtggatcacaaggtcaggagttcaagaccagcctggccaacatggtgaaaccccatctctactaaaaataaaaaactcagccaggcgtggtggcgggtgcctgtaatcccagctactcgggaggctgaagcaggagaattgcttaaaatcagaaggtggaggttgcagtgagctgagatcgtgtcactgcactccagcctgggcaaaagagcgaaactccatctcaaataaacaaacaaataaataacaaaaaacaaaaatgttgcattaaacttagttcttgtctctcctttccactcttattcttaaatctgaagctcatcgactaagtgaaatatttaaagaatatgataggccagcaagaagaagtattatgtagtaccatagttagtaaattcgtaaaaccttggaagccattatttggtcccacttgcaatttagtgtttttgaagtgtgtagcttcattcagatagctctttaaataattaaaatataaaagcaaacaacccaaactacctgactataaacaggaaaagttaaccctcaaagagagttcttgtgaattctctttatgctggcaaatagctctaggattaaaggcacattagggtttccttcagtttgtttattctaagcttttactgtgctttttactgaacaagtttctgatgtataaaacttgcatctgatttctttggaaatattttcacaaaagttattttaatcagtatttttacattgcctttccagtgtccagaagtgtttctaaacttagaaagtgacctatagttttttaaaattatgttttcctagaacgtgccaaattttgatttactctaacaatcagtacttttcttcagatgctttgttctgtttagaacaaaaatgcactatagtttttaaagaatcatgcatctttgggttggcccaggatcaaatttgatattgaataatttattccagggcagctttcataaacatacttcatagatgttgttttgaaatgtttctaaatatctaaaatcatttcaacagcagaaatgatttttattttaacaaaagattatgatagcccttgtagtgtttaaaagtggtcatatttattactgactttgagtcaggtgttaaaatagcagtgccacagctcgtctcttgccttagtgtgctgctgtgagagtcacagtggaaactgcagggaggaggtgtgttcctaagaaccaaaatccagcacagcatcctgtgaagccacgtgtaatgatggtcccataaggaaagtatgtgaatatggctcttgtaaaggattaactattgtaattttagcttatgctctgtattctgttttctatggaattatttaagcccttttagtgacctttgtcctggcccatttaaaaactaaaatgtagtatatattgtataaaatggaaatatcattattgcttcattaggggaaactgtacataggcattgaaagaagggtaaaagcaagcagttttatcaggcagttgtaaaacaccaaaaatatagattcgtctttgacgtgtaacacactaaatgtattttgtacagcatctggtttaaaaggtgccttaagagtttaccattacttgctttgttctatatacagattatgtccaatgtatcattttgaagtaaataaccttattttagtata

SEQ ID NO: 3 - protein, 532 aa, Mus musculus
MTQLYTYIRLLGACLFIISHVQGQNLDSMLHGTGMKSDLDQKKPENGVTLAPEDTLPFLKCYCSGHCPDDAINNTCITNGHCFAIIEEDDQGETTLTSGCMKYEGSDFQCKDSPKAQLRRTIECCRTNLCNQYLQPTLPPVVIGPFFDGSIRWLVVLISMAVCIVAMIIFSSCFCYKHYCKSISSRGRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDIPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 4 - DNA, 5481 bp, Mus musculus
ccgcgcgagacgacgactgtacggccgcgcgaggggcgaccgggcccgggccgctgcacgccgagggcggaggccgagccgggccccgccgccccgcggctgtccgtgcccgcccgcgccgagcgccggaggatgagtttctcgggatcccgatttatgaaaatatgcatcgctttgatactgtcttggaattcatgagatggaagcataggtcaaagctgttcggagaaattggaactacagttttatctagccacatctctgagaattctgaagaaagcagcaggtgaaagtcattgccaagtgattttgttctgtaaggaagcctccctcattcacttacaccagtgagacagcaggaccagtcattcaaagggccgtgtacaggacgcgtgcgaatcagacaatgactcagctatacacttacatcagattactgggagcctgtctgttcatcatttctcatgttcaagggcagaatctagatagtatgctccatggcactggtatgaaatcagacttggaccagaagaagccagaaaatggagtgactttagcaccagaggataccttgcctttcttaaagtgctattgctcaggacactgcccagatgatgctattaataacacatgcataactaatggccattgctttgccattatagaagaagatgatcagggagaaaccacattaacttctgggtgtatgaagtatgaaggctctgattttcaatgcaaggattcaccgaaagcccagctacgcaggacaatagaatgttgtcggaccaatttgtgcaaccagtatttgcagcctacactgccccctgttgttataggtccgttctttgatggcagcatccgatggctggttgtgctcatttccatggctgtctgtatagttgctatgatcatcttctccagctgcttttgctataagcattattgtaagagtatctcaagcaggggtcgttacaaccgtgatttggaacaggatgaagcatttattccagtaggagaatcattgaaagacctgattgaccagtcccaaagctctgggagtggatctggattgcctttattggttcagcgaactattgccaaacagattcagatggttcggcaggttggtaaaggccgctatggagaagtatggatgggtaaatggcgtggtgaaaaagtggctgtcaaagtgttttttaccactgaagaagctagctggtttagagaaacagaaatctaccagacggtgttaatgcgtcatgaaaatatacttggttttatagctgcagacattaaaggcactggttcctggactcagctgtatttgattactgattaccatgaaaatggatctctctatgacttcctgaaatgtgccacactagacaccagagccctactcaagttagcttattctgctgcttgtggtctgtgccacctccacacagaaatttatggtacccaagggaagcctgcaattgctcatcgagacctgaagagcaaaaacatccttattaagaaaaatggaagttgctgtattgctgacctgggcctagctgttaaattcaacagtgatacaaatgaagttgacatacccttgaataccagggtgggcaccaagcggtacatggctccagaagtgctggatgaaagcctgaataaaaaccatttccagccctacatcatggctgacatctatagctttggtttgatcatttgggaaatggctcgtcgttgtattacaggaggaatcgtggaggaatatcaattaccatattacaacatggtgcccagtgacccatcctatgaggacatgcgtgaggttgtgtgtgtgaaacgcttgcggccaatcgtgtctaaccgctggaacagcgatgaatgtcttcgagcagttttgaagctaatgtcagaatgttgggcccataatccagcctccagactcacagctttgagaatcaagaagacacttgcaaaaatggttgaatcccaggatgtaaagatttgacaattaaacaattttgagggagaatttagactgcaagaacttcttcacccaaggaatgggtgggattagcatggaataggatgttgacttggtttccagactccttcctctacatcttcacaggctgctaacagtaaaccttaccgtactctacagaatacaagattggaacttggaacttcaaacatgtcattctttatatatggacagctttgttttaaatgtggggtttttttgttttgctttttttgttttgttttggttttgatgcttttttggtttttatgaactgcatcaagactccaatcctgataagaagtctctggtcaacctctgggtactcactatcctgtccataaagtggtgctttctgtgaaagccttaagaaaattaatgagctcagcagagatggaaaaaggcatatttgccttctaccagagaaaacatctgtctgtgttctgtctttgtaaacagcctatagattatgatctctttgggatactgcctggtttatgatggtgcaccatacctttgatatgcataccagaattctctgctgccctagggcttagaagacaagaatgtaaaggttgcacaggaaggtatttgtggccagtggtttaaatatgcaatatctagttgacaatcgccaatttcataaaagccatccaccttgtaactgtagtaacttctccactgactttatttttagcataatagttgtgaaggccaaactccatgtaaagtgtccatagacttggactgttttcccccagtcaccattttgttctccttttggtaattatttttgttataaaaagccacctatccagaattggagctctctgtcttgaaccatactttgaaagaaacgcctcttccgtactgcatctgatcacaatgtgcatacctatgatcaaattctggagtctttgttctcggtacctcctaaaaaggaaagttgattcttgtgtaacatgcttttattttcagaacctgcacagctgtcattctagccatgttttacctacacactcagttctatacaagacagcccatacactctgtctcacatctgatccttggtgggaagtgttttaaagtagaactatgtatgaatttcagaattcatgcattttaaaacttcactaagatattgtctcatatctttatgagaatgtcagctgacttttcaactaacagtaaatgtattttagatatctaaatcttttgaaatttggttttacaatttctggtccctaattgtgaagacaagaggcagaagtacccagtcactacccatatttacactgaacgttattaaataaaatgatgtgtattttattataaaataaatataggccttgttatctcaaaaaacagatctggttcaaacttattataccaatatcatactatttaaatgttctaagtaaacaagccatgtgagcatcaagtggcattggctctttggatgaaacataaacttaaggtgattgtatcaacacatagagtgactgaaattaaatgggaggcaggtagagcatatgtccatctgtccacctacaggcatgactaaactacagctcatattccacaaatttgagatttgtcttgcctggtttgtttagtgagtctcatctgatgtacctaaagcctgagagtactgaggtctgattttatatctttcctgaataaactaaatcttttttgtcacttatcatcttaatgatatacctaaggaataattctttggcatgtttcagttgtgtgtggcagccactgtaatgactcttctctaagaaaggctgtcaggagttaattataaggcaggcagtgagcgctctagtcactgccttcccacgctgccatcactgcattcatgggaatcagtgacgttctcgaaatggcaaacgctgctgcttttccttatttggaatcctaaaatcaaaagttgcattaaacttactgtgttctcttctccctctcagccataaatgtaaaattcagtaagtaaaaatatttaaagagtgtatcagccctttggccagtgagatagctcagtagataaaggcatttgctgccaagtcctcaacctcaattcagactctgggggacacatggcgaagggaggagccaactaccccatcattgtcctctgacttccacacactccatggcttgcgcccctccccacagacacacaccatgtactccacaaaagtagtttaaaggaaaaaaagaatagaacccactgtgtaatggaataagtattatgtagttacttaacaacttgtaaaaatctggaaactatgatttggttcccctttgaatctagagtttaaaaaacagatggctaaaatcagccatcatttaaataattaaaaataaaagccccaaaccccaaactgcctaaataaataccaagtaatccaggaagccgtcatgtgtggtttgtatgaccagtagttctctggtacagagcatgttaagatttgccccagcctgattctctgaggtctctgcattactgagtactgttctgagtataaaatctgaactgatttctctagaaatactgtaacaaaaaggtattttagtcagcatgttatgttaacccttccactgtctagaaacttgaataagcacataaagacaccttttgctgtcatcatctgttgtcctggaatgtgccagttttaatttattcattctaatgatattcaatttgcttttcttttttagatgtttttcttgtttagagtaaaaggacgaatttttcaagaaccttgcatctctgatttggcctaaggtcaaattggatattgagtagtctattccagggcagatttcctaagcaatacttgtcttttcagctatgtattgttttgaaatgtttccatttcaacagaggtgtaagtcatgtgaaaagaaaggtggtgtagcccttgtggtaatgacacaagttgacttgcgtcagatgttaagcagggacagttctcccacctcctggctgtaaggagtggaaactaggcaagcagtgtatcagtccacagaggacaggaagggtcatcccataaagaaagcctgtgagtatggctttggcaaaaaattagacataatactgtccttttaggttgtgctctgttctttcctttcagtggaattatttaagctctttagtggcctttgtttttcccacttaaaaactaaaatgtagcatatattgtataaaatggaaatattaatagcttagggaaactgtacataaggcattgacaggtttaaaaaaagcatttttattatgcagttgtaaaacaccaaaaatatagattcatcttgatatgtaacactaagtgtattttgtacagcatctgatttgaaaggtgccttatgaagtttaccattaattgctttgttctatatacagattatgtccaatgtatcatttttcagtaaataaccttattttagta

(19) United States
(12) Patent Application Publication
(10) Pub. No.: US 2024/0124845 A1
(43) Pub. Date: Apr. 18, 2024

(54) SKELETAL STEM CELL ISOLATION AND USES THEREOF

(71) Applicant: University of Rochester, Rochester, NY (US)

(72) Inventors: Wei Hsu, Cambridge, MA (US); Takamitsu Maruyama, Cambridge, MA (US)

(73) Assignee: University of Rochester, Rochester, NY (US)

(21) Appl. No.: 18/547,438

(22) PCT Filed: Feb. 22, 2022
(86) PCT No.: PCT/US22/17264

§ 371 Date: Aug. 22, 2023

Related U.S. Application Data
(60) Provisional application No. 63/154,293, filed on Feb. 26, 2021.

(51) Int. Cl. C12N 5/0775

(52) U.S. Cl. CPC C12N 5/0668; C12N 2500/25; C12N 2501/11; C12N 2501/115; C12N 2501/30; C12N 2509/00; C12N 2513/00; C12N 2539/00

(57) ABSTRACT

This application relates to stem cell biology and regenerative medicine. Disclosed herein methods for isolation of skeletal stem cells, related methods, related compositions, related products, and related uses.

### CROSS REFERENCE TO RELATED APPLICATION

This application claims priority to U.S. Provisional Application No. 63/154,293 filed on Feb. 26, 2021. The content of the application is incorporated herein by reference in its entirety.

### GOVERNMENT INTERESTS

This invention was made with government support under DE015654 and DE026936 awarded by the National Institutes of Health. The government has certain rights in the invention.

### FIELD OF THE INVENTION

This invention relates to stem cell biology and regenerative medicine.

### BACKGROUND

Skeletal stem cells (SSCs) are a type of self-renewing, multipotent, and skeletal lineage-committed progenitor cells that are capable of giving rise to cartilage, bone, and bone marrow stroma including marrow adipocytes and stromal cells. With the “trilineage” potentials to differentiate into chondrocytes, osteoblasts, marrow stromal cells, or adipocytes, SSCs play important roles in the development, homeostasis, and regeneration of bone tissues. For example, SSCs from the suture mesenchyme, which are referred to as suture stem cells (SuSCs), exhibit long-term self-renewal, clonal expansion, and multipotency. These SuSCs reside in the suture midline and serve as the skeletal stem cell population responsible for calvarial development, homeostasis, injury repair, and regeneration. Yet, the in vivo identity of SuSCs and SSCs remains largely elusive. Presently, the lack of a cell surface marker for such stem cell isolation and the inability to maintain stemness characteristics ex vivo are two critical hurdles that restrict further advances in the field of skeletal regeneration. Thus, there is a need for a cell surface marker, related cell identification and isolation methods, and in vitro or ex vivo maintaining methods.

### SUMMARY

This application addresses the need mentioned above in a number of aspects. In one aspect, the present application provides a method for identifying or isolating, or enriching a skeletal stem cell or a suture stem cell. The method comprises obtaining a start cell population identifying from the start cell population a cell expressing a BMPR1A polypeptide (BMPR1A⁺ cell), or expressing an mRNA encoding the BMPR1A polypeptide. In one embodiment, the method further comprises isolating the identified BMPR1A⁺ cell. In one embodiment, the method further comprises collecting a plurality of BMPR1A⁺ cells to obtain an enriched suture stem cell population or an enriched skeletal stem cell population.

In the methods described above, the cells can express one or more markers selected from the group consisting of Axin2, BMP2, BMP3, BMP4, BMP6, BMP7, BMP8b, and BMP15. The cells can also be positive for one or more positive markers selected from the group listed in Table 1 below. The cells can also be negative for one or more negative markers selected from the group listed in Table 2 below.

In some embodiments, the BMPR1A⁺ cell or cells can be isolated using a protein, a polypeptide, or a composition that specifically binds to BMPR1A. In one embodiment, the protein comprises an anti-BMPR1A antibody or an antigen-binding fragment thereof. In another embodiment, the protein, peptide, or composition comprises a ligand of BMPR1A or a BMPR1A-binding fragment thereof. Examples of the ligand include BMP2, BMP4, BMP6, BMP7, and GDF6. The BMPR1A⁺ cell or cells can be identified, isolated, or enriched by fluorescence activated cell sorting.

In the methods described herein, the start cell population can be from a tissue of a subject, such as a vertebrate, a mammal (including human and non-human mammal). The start cell population can comprise one or more selected from the group consisting of bone marrow, cord blood cells, embryonic stem cells or progenies thereof, mesenchymal stem cells or progenies thereof, and induced pluripotent stem cells (iPSCs) or progenies thereof. In a preferred embodiment, the mesenchymal stem cells are suture mesenchymal stem cells.

In a second aspect, the present application provides a method for maintaining suture stem cells or skeletal stem cells in vitro. The method comprises (i) providing a population of suture stem cells or skeletal stem cells; (ii) seeding the cells in a maintaining medium on a low attachment surface or ultra-low attachment surface; and (iii) culturing the cells or the progenies thereof for a period of time to form one or more spheres.

The population can be from a tissue of a subject, such as a vertebrate, a mammal (including human and non-human mammal). The period of time can be 5-20 days, such as 5-15 days and 7-10 days. The maintaining medium can comprise (i) a culture or nutrient medium and (ii) one or more of the following: about 5-125 μg/ml insulin, about 20-500 μg/ml transferrin, about 4-100 nM progesterone, about 5-150 nM sodium selenite, about 10-300 nM putrescine, about 4-100 ng/ml EGF, about 4-100 ng/ml bFGF, and about 4-100 ng/ml B27 supplement. The method for maintaining the sternness can further comprise: (iv) obtaining cells from a sphere formed from step (iii) described above, and then (v) repeating steps (ii)-(iii) described above.

In one embodiment, the maintaining medium comprises one or more of the following: about 10-50 μg/ml insulin, about 50-200 μg/ml transferrin, about 10-50 nM progesterone, about 10-100 nM sodium selenite, about 10-100 nM putrescine, about 10-50 ng/ml EGF, about 10-50 ng/ml bFGF, and about 10-50 ng/ml B27 supplement. In a further embodiment, the maintaining medium comprises one or more of the following: about 25 μg/ml insulin, about 100 μg/ml transferrin, about 20 nM progesterone, about 30 nM sodium selenite, about 60 nM putrescine, about 20 ng/ml EGF, about 20 ng/ml bFGF, and about 20 ng/ml B27 supplement. In yet another embodiment, the maintaining medium comprises about 1-30% FBS, such as about 5-25% and about 20%. The suture stem cells or skeletal stem cells comprise BMPR1A⁺ cells. The cells can be seeded in single cell suspension. To that end, the cells can be seeded at a density of about 500 cells/ml to about 10,000 cells/ml.

The present application also provides a sphere of cells comprising one or more BMPR1A⁺ cells. The sphere can be 20 to 200 μm in diameter, or have about 10 to 500 (e.g., 50-400, 100-300, etc.) cells per sphere. The sphere can comprise Axin2+ cells.

The present application further provides a composition comprising (i) a carrier and (ii) one or more BMPR1A⁺ cells, or one or more spheres described above, or cells (such as progeny cells) derived from the one or more spheres. The composition can be a pharmaceutical composition where the carrier is a pharmaceutically acceptable carrier. The composition can be an in vitro cell culture composition and the carrier can comprise a culture medium or a maintaining medium. The present application also provides a bone regeneration product or formulation comprising (i) the composition described above and (ii) a scaffold.

Also provided is a method for generating or regenerating cartilage or bone in a subject. The method comprises administering to a subject in need thereof an effective amount of the composition described above or the bone regeneration product or formulation described above at a site where regeneration of bone or cartilage is desired.

Further provided is a method of generating skeletal, stromal, or cartilaginous tissue. The method includes (i) providing one or more BMPR1A⁺ cells described above, or one or more spheres described above, or cells (such as progeny cells) derived from the one or more spheres described above, and (ii) inducing differentiation of the BMPR1A⁺ cells, or of the cells from the one or more spheres.

The details of one or more embodiments of the present application are set forth in the description below. Other features, objectives, and advantages of the present application will be apparent from the description and from the claims.

### BRIEF DESCRIPTION OF THE DRAWINGS

FIGS. 1A-1G show that stem cell-mediated calvarial development and homeostasis require Bmpr1a. Bmpr1a^{Ax2}, Bmpr1a^{-/-}, and Acvr1^{Ax2} mouse models examine BMP type I receptors in calvarial morphogenesis. (A) Diagram illustrates the procedure for inducing Bmpr1a or Acvr1 deletion during calvarial development in Axin2+ SuSCs using Axin2^{Cre-Dox} (Axin2-rtTA; TRE-Cre) mice. Control mice were Axin2-rtTA; Bmpr1a^{Fx/Fx} or TRE-Cre; Bmpr1a^{Fx/Fx} mice with Dox for Bmpr1a^{Ax2} mice, Bmpr1b^{+/-} mice for Bmpr1b^{-/-} mice, Axin2-rtTA; Acvr1^{Fx/Fx} or TRE-Cre; Acvr1^{Fx/Fx} mice with Dox for Acvr1^{Ax2} mice. Mice were examined at 2 months old (n=3 mice per group). (B) Photographs of mouse heads and faces. (C) μCT images of skulls of the indicated mice. Arrowheads indicate aberrant suture closure. (D) Hematoxylin and eosin staining of calvarial tissue at the sagittal suture. Scale bars, 400 (E) Diagram illustrates the deletion of Bmpr1a in SuSCs in mice after calvarial development. Control mice were Axin2-rtTA; Bmpr1a^{Fx/Fx} or TRE-Cre; Bmpr1a^{Fx/Fx} mice with Dox. Mice were examined after 3 months (n=3 mice per group). (F) μCT images of skulls of the indicated mice. Arrowheads indicate aberrant suture closure. (G) Hematoxylin and eosin staining of calvarial tissue at the sagittal and lambdoid sutures. Asterisks indicate aberrant suture closure. Scale bars, 200 μm. COR, coronal suture; LAM, lambdoid suture; SAG, sagittal suture.

FIGS. 2A and 2B show that craniosynostosis caused by SuSC-specific disruption of Bmpr1a involves an unusual suture closure process. Bmpr1a^{Ax2} mice (Axin2-rtTA; TRE-Cre) were induced with Dox from E16.5-P3. Control mice were Axin2-rtTA; Bmpr1a^{Fx/Fx} or TRE-Cre; Bmpr1a^{Fx/Fx} mice with Dox. Skulls of mice of the indicated conditions were analyzed at P0, P7, or P14 (n=3 mice per group). (A) Alizarin red staining of intact skulls. (B) Goldner's trichome staining of calvarial sections along sagittal suture. Arrowheads indicate mineralization between calvarial bone plates. Arrows indicate osteogenic fronts. Scale bar, 400 μm.

FIGS. 3A-3D show that the loss of Bmpr1a in SuSCs leads to aberrant intramembranous ossifications within the suture mesenchyme. Bmpr1a^{Ax2} mice (Axin2-rtTA; TRE-Cre) were induced with Dox from E16.5-P3. Control mice were Axin2-rtTA; Bmpr1a^{Fx/Fx} or TRE-Cre; Bmpr1a^{Fx/Fx} mice with Dox. Calvarial sections were analyzed at mice at either P0 or P3 as indicated (n=3 mice per group). (A) Ki67 immunostaining at the osteogenic front (OF, indicated by dotted lines). (B) Ki67 immunostaining at the suture mesenchyme. In A and B, scale bars, 60 μm. (C) Osterix (Osx) immunostaining. Scale bars in upper 4 images, 400 μm; scale bars in lower 2 images, 100 μm. (D) In situ hybridization of type 1 collagen (Col1). Scale bars, 200 μm. In C and D, arrows indicate osteogenic fronts.

FIGS. 4A-4D show that Bmpr1a regulates SuSCs and stem cell-dependent bone formation. (A-C) Kidney capsule transplantation with limiting dilution analysis of control and Bmpr1a^{Ax2} cells, isolated from the P5 suture mesenchyme of mice administered Dox from E16.5 to P3, to examine SuSC frequency (n=3 mice per group). Control mice were Axin2-rtTA; Bmpr1a^{Fx/Fx} or TRE-Cre; Bmpr1a^{Fx/Fx} mice with Dox. Ectopic bone formation is assessed by von Kossa staining in whole mounts (A) and histology in sections (B). Scale bars, 4 mm (A); 200 μm (B). Quantification of bone formation rate with transplantation of 10⁵, 10⁴, 10³, and 10² cells with a quantitative estimation for stem cell frequency using ELDA software (C). (D) Immunostaining for Axin2 of sections of the P7 sagittal suture with quantification of the average percentage of Axin2+ cells. Sections were counterstained with DAPI. Broken lines define the calvarial bones (scale bars, 100 μm). Quantified data are from 3 mice per group and are presented as mean±SEM (*, p<0.01 by student t-test).

FIGS. 5A-5D show that SuSC stemness is preserved in sphere culture. (A, B) Genetic cell-labeling with the Axin2^{Cre-Dox}; R26RTomato mouse model was used to trace the fate of Axin2+ SuSCs in sphere culture. Dox was administered to Axin2^{Cre-Dox}; R26RTomato mice from P7-P10. Panel A shows fluorescent images of Axin2+ SuSCs on day 1 of culture and day 14 of culture. Scale bars, 100 μm. Panel B shows the evaluation of the proportion of spheres derived from Axin2+ cells (arrows) and Axin2− cells (arrowhead) and the proportion of spheres derived from Axin2+ and Axin2− cells was quantified (p<0.01, n=3, mean±SEM, student t-test). Scale bar, 400 μm. (C) Whole-mount imaging of Tomato fluorescence of (Axin2+-derived) SuSC spheres 4 weeks after transplantation into the kidney capsule. Scale bars, 1 mm. (D) Whole-mount von Kossa staining of ectopic bones generated by SuSC spheres 8 weeks after transplantation. (E) Comparison of ectopic bone grown from SuSC spheres 8 weeks after transplantation into a kidney capsule (left) and calvarial bone plate from 2-month mouse skull (right). Sections were stained with hematoxylin and eosin. Scale bars, 200 μm. In C-E, images are representatives of three independent experiments.

FIGS. 6A-6N show that Bmpr1a is essential for SuSC self-renewal. (A-C) Ex vivo pulse-chase labeling analysis of cells isolated from Axin2^{GFP} mouse sutures labeled in vivo from P7-P10 by the administration of Dox and then cultured in the absence of Dox. Spheres from 1⁰, 2⁰, and 3⁰ cultures were examined for the presence of cells with GFP fluorescence (arrows). (D) Graph of the percentage of spheres with (Axin2+) or without (Axin2−) the label-retaining GFP+ cell in the indicated passages. (E) Diagrams illustrating the distribution of GFP intensity in cells generated by the asymmetric or symmetric division of SuSCs. (F-G) Ex vivo pulse-chase labeling of Axin2-positive cells (as described for A-C) was followed by immunostaining for Axin2 (F) or incubation of cells with EdU to label proliferating cells (G) in the suture spheres. Arrows indicate a single GFP+ label-retaining cell that is positive for Axin2 immunofluorescence (F) or is negative for EdU labeling (G). In F, scale bars, 100 μm; in G, scale bars, 50 μm. (H) Ex vivo pulse-chase labeling of Axin2-positive cells (as described for A-C) was followed by immunostaining for Bmpr1a in the suture spheres. Arrows indicate a single GFP+ label-retaining cell that is positive for Bmpr1a. Scale bars, 50 μm. (I, J) In vitro self-renewal was examined by serial culturing of spheres and sphere number (I) and size (J) were evaluated in spheres from suture cells from control mice or Bmpr1a^{Ax2} mice. For sphere number, the data are presented as mean±SEM (n=3; *, p<0.05, student t-test). For sphere size, individual spheres are shown with the average (middle line), 75% tile (top line), and 25% tile (bottom line) values. Statistical significance was determined by the two-sided student t-test [n values: 1⁰ control, 236 spheres, and Bmpr1a^{Ax2}, 188 spheres; 2⁰ control, 124 spheres, and Bmpr1a^{Ax2}, 66 spheres (N=3 independent experiments)]. (K-N) Kidney capsules were transplanted with the 1⁰ spheres cultured from control or Bmpr1a^{Ax2} cells, isolated from the P5 suture mesenchyme of mice administered Dox from E16.5 to P3 (n=3 mice per group). Control mice were Axin2-rtTA; Bmpr1a^{Fx/Fx} or TRE-Cre; Bmpr1a^{Fx/Fx} mice with Dox. Tissue was evaluated by whole-mount von Kossa staining (K-L) and histological (M-N) analyses. In K and L, scale bars, 2 mm; in M and N, scale bars 800 μm.

FIGS. 7A-7K show that self-renewal and osteogenic ability of human SuSCs. (A-C) Sections of the 14-month-old human coronal suture were examined by hematoxylin and eosin (H&E) staining (A) or by immunostaining for AXIN2 (B) or BMPR1A (C). Broken lines define the calvarial bones at the osteogenic front (OF). Scale bars, 500 μm in main images and 100 μm in enlarged images from B and C. Images are representative of ≥5 individuals. (D-F) Characterization of spheres formed by cells isolated from human suture tissue. A representative primary sphere is shown in (D). Scale bar, 100 μm. Graphs of average number (E; n=5, mean±SD) and size (F; n=5, >15 spheres in each passage, mean±SD) of spheres formed by the indicated culture of human suture cells starting with 10⁴ cells for each passage. (G-I) Immunostaining for AXIN2 (arrow) and EdU labeling of a human sphere. Scale bars, 100 μm. (J, K) Kidney capsules were transplanted with the 30 primary human spheres and evaluated by von Kossa staining of whole-mounts (J) and sections (K). In J, scale bar, 300 μm; in K, scale bar 100 μm. Data in G-K are representatives of at least five independent experiments.

FIGS. 8A-8L show that the osteogenic ability of mouse Bmpr1a-positive and human BMPR1A-positive suture cells. (A) Cell sorter isolation of Bmpr1a/BMPR1A^{High} and Bmpr1a/BMPR1A^{Low} cell populations from P10 mouse or human suture mesenchymes. (B-F) Sorted mouse cells (5×10³) were transplanted into kidney capsules and evaluated by von Kossa (VK) staining to identify bone tissue or immunostaining for Osterix (Osx) to identify osteoprogenitor cells. (G-L) Sorted human cells (5×10³) were transplanted into kidney capsules and evaluated by von Kossa (VK) staining to identify bone tissue or immunostaining for Osterix (Osx) to identify osteoprogenitor cells. In L, tissue was counterstained with DAPI and the dotted area represents bone. Images are the representatives of at least five independent experiments. Scale bars, 50 μm (B-C, H-M), 100 μm (D-F).

FIG. 9A-9C show mouse genetic models for the identification of Axin2-expressing cells and their derivatives. (A) Schematic of the genetic cell-labeling strategy for identifying the Axin2-expressing cells by GFP analysis. (B) Schematic of the genetic cell-labeling strategy to identify cells descending from Axin2-expressing cells. (C) The fluorescence image shows GFP analysis of the Axin2-rtTA; TRE-H2BGFP mice at P9, which reveals the Axin2-expressing cells in the midline of the suture mesenchyme (n=3). The fate of these cells can be traced using the cell tracing models based on (3-galactosidase 03-gal) positivity (Axin2^{Cre-Dox}; R26RlacZ) or by red fluorescence from Tomato (Axin2^{Cre-Dox}; R26RTomato). Broken lines define the calvarial bones and osteogenic fronts. Scale bar, 100 μm.

FIGS. 10A-10E. Enhanced BMP signaling in suture stem cells. A) Gene expression profiles of Axin2 positive (+) and negative (−) cells isolated from suture mesenchyme analyzed by active Z score using IPA software to predict the activity of signaling pathways. (B) Heatmap showing the expression of genes associated with BMP pathways—7 ligands, the receptor subunit Bmpr1a, and 2 negative regulators Smad7 and Smurf1—in Axin2+ and Axin2− suture cell populations. (C-E) Section of the sagittal suture expressing lacZ in Axin2+ cells was analyzed at postnatal (P) day 28. Sections were processed for β-gal activity and Bmpr1a immunoreactivity and counterstained with DAPI. In C, Axin2-expressing cells were identified with Axin2^{Cre-Dox}; R26RlacZ by staining for β-gal. In D, cells were stained with an antibody recognizing Bmpr1a, and nuclei were labeled with DAPI. In E, both β-gal and Bmpr1a positivity is shown. Broken lines define the calvarial bones at the osteogenic front (OF). Images are representatives of 3 independent experiments. Scale bars, 100 μm.

FIGS. 11A-11D. The efficacy of the Cre-mediated ablation of Bmpr1a and Bmpr1a antibody specificity. Dox was administered from E16.5 to P3 to activate Cre expression in the Axin2+ SuSCs. Sagittal suture sections were obtained at P7. (A) Absence of LacZ expression in calvarium labeled with β-gal from TRE-Cre; R26RlacZ mice. (B) Presence of LacZ expression in calvarium labeled with β-gal from Axin2^{Cre-Dox}; R26RlacZ mice. In A and B, sections were counterstained with nuclear fast red. (C, D) Bmpr1a staining of control and Bmpr1a^{Ax2} calvarium. Sections were counterstained with DAPI. Broken lines define the calvarial bones and osteogenic fronts. Scale bars, 100 μm.

FIGS. 12A-12F. Effects of Bmpr1a loss of function on the skull. Dox was administered from E16.5 to P3 to activate Cre expression in the Axin2+ SuSCs. Control mice were Axin2-rtTA; Bmpr1a^{Fx/Fx} or TRE-Cre; Bmpr1a^{Fx/Fx} mice with Dox for Bmpr1a^{Ax2} mice, Bmpr1b^{+/-} mice for Bmpr1b^{-/-} mice, and Axin2-rtTA; Acvr1^{Fx/Fx} or TRE-Cre; Acvr1^{Fx/Fx} mice with Dox for Acvr1^{Ax2} mice. n=4 mice per group. (A, B) Graphs of skull length and width at P28 of control mice or the indicated mutants. (C, D) Graphs of skull length and width of control mice or the Bmpr1a^{Ax2} mutant at the indicated postnatal days. In A-C, statistical significance was determined by student t-test (n=4; *, p<0.05). (E, F) μCT images of the skulls of control and Bmpr1a^{Ax2} mutant mice at 2 months old. Scale bars, 2 mm.

FIG. 13A-13H show multiple suture synostoses in skulls of mice with SuSC-specific deletion of Bmpr1a. Dox was administered from E16.5 to P3 to activate Cre expression in the Axin2+ SuSCs. Control mice were Axin2-rtTA; Bmpr1a^{Fx/Fx} or TRE-Cre; Bmpr1a^{Fx/Fx} mice with Dox. n=3 mice per group. Skulls from 2-month-old mice of the indicated genotypes were evaluated. (A, B) Whole-mount skeletal staining with Alizarin red. Scale bars, 2 mm (C-H) Sections were stained with hematoxylin and eosin. Scale bars, 200 μm. Arrowheads and asterisks indicate examples of calvarial bone fusion. COR, coronal; LAM, lambdoidal; SQU, squamosal.

FIG. 14 shows identification by μCT analysis of multiple suture synostosis in skulls of mice with SuSC-specific deletion of Bmpr1a. Dox was administered from E16.5 to P3 to activate Cre expression in the Axin2+ SuSCs. Control mice were Axin2-rtTA; Bmpr1a^{Fx/Fx} or TRE-Cre; Bmpr1a^{Fx/Fx} mice with Dox. n=3 mice per group. Images are shown as 3D reconstructions (blue background) and as 2D images (black backgrounds) for the indicated sections of skulls from 2-month-old control and Bmpr1a^{Ax2} mice. Asterisks indicate examples of aberrant suture closure. AF, anterior frontal; FN, frontonasal; IN, internasal; LAM, lambdoid; NM, nasomaxillary; SAG, sagittal; SQU, squamosal.

FIG. 15 shows chondrocyte analysis in Bmpr1a^{Ax2} mice. For Bmpr1a^{Ax2} mice, Dox was administered from E16.5 to P3 to activate Cre expression in the Axin2+ SuSCs. Control mice were Axin2-rtTA; Bmpr1a^{Fx/Fx} or TRE-Cre; Bmpr1a^{Fx/Fx} mice with Dox. n=3 mice per group. Sagittal suture sections were obtained from the indicated mice and stained for type II collagen (Col2) and counterstained with DAPI. Sections were obtained at P3 for the control and Bmpr1a^{Ax2} mice, and at P7 for the Axin2^{-/-}; Fgfr1^{+/-} mice. Broken lines define the calvarial bones and osteogenic fronts. Scale bars, 100 μm.

FIGS. 16A-16C show the effect of Bmpr1a ablation on signaling pathways in the developing suture. Dox was administered from E16.5 to P3 to activate Cre expression in the Axin2+ SuSCs. Control mice were Axin2-rtTA; Bmpr1a^{Fx/Fx} or TRE-Cre; Bmpr1a^{Fx/Fx} mice with Dox. n=3 mice per group. Calvarial sections were obtained at P3. (A) Immunostaining of phosphorylated Smad1, Smad5, and Smad8 with antibodies recognizing all 3 phosphorylated Smads (pSmad1/5/8). Lower images are enlargements of the boxed areas in the upper images. Scale bars in upper images, 100 μm; scale bars in lower images, 50 μm. (B) Immunostaining of phosphorylated Tak1. Scale bars, 100 μm. (C) Immunostaining of the active form of the indicated mitogen-activated protein kinase (MAPK) family member: pJNK, pP38, and pErk. Scale bars, 200 μm. All images were counterstained with DAPI. Arrows or broken lines indicated the osteogenic front.

FIGS. 17A-17S show ex vivo culture and differentiation of SuSCs. (A) Schematic representations illustrate the isolation of suture cells from the mouse sagittal suture, followed by sphere culture in primary (1⁰), secondary (2⁰), tertiary (3⁰), and up to 4⁰-5⁰ passages. (B) Image of suture cells before sphere culture. Scale bar, 50 (C-E) Representative images of sphere cultures at the indicated passages. Scale bars, 50 (F-K) Time-course analysis of sphere formation from day 1 to day 12 of culture. Scale bars, 50 (L) Quantification of sphere size at the indicated passages (n=3, >150 spheres in each passage, mean+SD). (M-O) Differentiation of 3⁰ sphere cells into ALP+ osteoblasts (M), mineralized nodules positive for von Kossa staining (N), and alcian blue-positive chondrocytes (O). Scale bars, 100 μm. (P-S) Whole-mount and sections stained with von Kossa of kidney capsules transplanted with 3⁰ spheres or control untransplanted kidney capsule. Arrows indicate ectopic bone formation. In P and Q, scale bars are 250 μm; in R and S, scale bars are 100 μm.

FIGS. 18A-18G. Lentiviral gene delivery to examine the loss of Bmpr1a in sphere-mediated bone formation. (A) Cells isolated from the suture mesenchyme of Bmpr1a^{Fx/Fx} mice were infected by a lentivirus expressing red fluorescent protein (RFP) at 0, 1, and 5 MOI. Infection efficiency was monitored by fluorescent imaging. Scale bars, 1 mm. (B, C) Analysis of the sphere size (B) and number (C) at the indicated MOI (n=3, mean+SD). (D) The efficacy of Cre-mediated recombination was examined by PCR [primers: 5′-ggtttggatcttaaccttagg-3′ and 5′-tggctacaatttgictcatgc-3′], generating a 180 bp product. The unmodified Bmpr1aFx allele was detected by PCR [primers: 5′-gcagctgctgctgcagcctcc-3′ and 5′-tggctacaatttgictcatgc-3′], generating a 230 bp product. (E-G) Whole-mount von Kossa staining of ectopic bone formation in the kidney capsule transplanted with Bmpr1a^{Fx/Fx} 1⁰ spheres infected with lentivirus-expressing GFP (E; Lenti-GFP) or Cre (F; Lenti-Cre). Quantitative evaluation of the size of the von Kossa-stained area (asterisk, p<0.05, n=3, mean±SEM, student t-test) (G). Images are representatives of three independent experiments. Scale bars, 2 mm (D-E).

FIGS. 19A-19E show the identification of cells positive for AXIN2 and BMPR1A in the human lambdoid suture. Sections of the 10-month-old human lambdoid suture were examined. (A) Hematoxylin and eosin (H&E) staining. Scale bar, 400 μm. (B-E) Immunostaining of AXIN2 or BMPR1A. Panels C and E show the enlargement of the boxed regions in panels B and D. Broken lines define the calvarial bones. In B and D, scale bars, 400 μm; in C and E, scale bars, 100 μm.

FIGS. 20A-20C show the purification of mouse and human Bmpr1a/BMPR1A-expressing cells. (A) The specificity of the antibody used for cell sorting was determined by FACS of wild-type (WT) and Bmpr1a^{Ax2} suture cells from P10 mice. (B, C) Cell sorting of Bmpr1a/BMPR1A^{High} and Bmpr1a/BMPR1A^{Low} cell populations from mouse (B) and human (C) suture mesenchyme. Human suture mesenchyme was isolated from 4 to 12-month patients. FACS was performed with (top row) and without (bottom row) the antibody addition. Data are representatives of three independent experiments. Approximately 5-6% of suture cells have high amounts of Bmpr1a/BMPR1A in mice/humans.

### DETAILED DESCRIPTION OF THE INVENTION

This application relates to stem cell biology and regenerative medicine. Certain aspects of this invention are based, at least in part, on the identification and use of one or more cell surface markers of SSCs and/or SuSCs. The ability of SSCs and/or SuSCs to engraft in injury sites to replace the damaged skeleton can be used for stem cell-based therapy.

### Cells and Markers

Skeletal stem cells (SSCs) from the suture mesenchyme, also referred to as suture stem cells (SuSCs), exhibit long-term self-renewal, clonal expansion, and multipotency. These SuSCs reside in the suture midline and serve as the skeletal stem cell population responsible for calvarial development, homeostasis, injury repair, and regeneration. The ability of SuSCs to engraft in injury site to replace the damaged skeleton support their potential use for stem cell-based therapy.

As disclosed herein, BMPR1A was identified as essential for SuSC self-renewal and SuSC-mediated bone formation. In addition, SuSC-specific disruption of Bmpr1a in mice caused precocious differentiation, leading to craniosynostosis initiated at the suture midline, which is the stem cell niche. Shown below are exemplary amino acid and nucleic acid sequences for human or mouse BMPR1A.

BMPR1A Sequences:

| Name | Sequence |
|---|---|
| Protein | MPQLYIYIRLLGAYLFIISRVQGQNLDSMLHGTGMKSDSDQKKSENGVTLAPEDTLP |
| sequence of | FLKCYCSGHCPDDAINNTCITNGHCFAIIEEDDQGETTLASGCMKYEGSDFQCKDSP |
| human BMPR1A | KAQLRRTIECCRTNLCNQYLQPTLPPVVIGPFFDGSIRWLVLLISMAVCIIAMIIFS |
| (SEQ ID NO: | SCFCYKHYCKSISSRRRYNRDLEQDEAFIPVGESLKDLIDQSQSSGSGSGLPLLVQR |
| 1) NCBI | TIAKQIQMVRQVGKGRYGEVWMGKWRGEKVAVKVFFTTEEASWFRETEIYQTVLMRH |
| Reference | ENILGFIAADIKGTGSWTQLYLITDYHENGSLYDFLKCATLDTRALLKLAYSAACGL |
| Sequence: | CHLHTEIYGTQGKPAIAHRDLKSKNILIKKNGSCCIADLGLAVKFNSDTNEVDVPLN |
| NP_004320.2 | TRVGTKRYMAPEVLDESLNKNHFQPYIMADIYSFGLIIWEMARRCITGGIVEEYQLP |
| | YYNMVPSDPSYEDMREVVCVKRLRPIVSNRWNSDECLRAVLKLMSECWAHNPASRLT |
| | ALRIKKTLAKMVESQDVKI |
| Nucleic acid | aagagtcggcggcggtggcggcggccgctgcagagattggaatccgcctgccgggct |
| sequence | tggcgaaggagaagggaggaggcaggagcgaggagggaggagggccaagggcgggca |
| encoding human | ggaaggcttaggctcggcgcgtccgtccgcgcgcggcgaagatcgcacggcccgatc |
| BMPR1A (SEQ ID | gaggggcgaccgggtcggggccgctgcacgccaagggcgaaggccgattcgggcccc |
| NO: 2) NCBI | acttcgccccggcggctcgccgcgcccacccgctccgcgccgagggctggaggatgc |
| Reference | gttccctggggtccggacttatgaaaatatgcatcagtttaatactgtcttggaatt |
| Sequence: | catgagatggaagcataggtcaaagctgtttggagaaaatcagaagtacagttttat |
| NM_004329.3 | ctagccacatcttggaggagtcgtaagaaagcagtgggagttgaagtcattgtcaag |
| | tgcttgcgatcttttacaagaaaatctcactgaatgatagtcatttaaattggtgaa |
| | gtagcaagaccaattattaaaggtgacagtacacaggaaacattacaattgaacaat |
| | gcctcagctatacatttacatcagattattgggagcctatttgttcatcatttctcg |
| | tgttcaaggacagaatctggatagtatgcttcatggcactgggatgaaatcagactc |
| | cgaccagaaaaagtcagaaaatggagtaaccttagcaccagaggataccttgccttt |
| | tttaaagtgctattgctcagggcactgtccagatgatgctattaataacacatgcat |
| | aactaatggacattgctttgccatcatagaagaagatgaccagggagaaaccacatt |
| | agcttcagggtgtatgaaatatgaaggatctgattttcagtgcaaagattctccaaa |
| | agcccagctacgccggacaatagaatgttgtcggaccaatttatgtaaccagtattt |
| | gcaacccacactgccccctgttgtcataggtccgttttttgatggcagcattcgatg |
| | gctggttttgctcatttctatggctgtctgcataattgctatgatcatcttctccag |
| | ctgcttttgttacaaacattattgcaagagcatctcaagcagacgtcgttacaatcg |
| | tgatttggaacaggatgaagcatttattccagttggagaatcactaaaagaccttat |
| | tgaccagtcacaaagttctggtagtgggtctggactacctttattggttcagcgaac |
| | tattgccaaacagattcagatggtccggcaagttggtaaaggccgatatggagaagt |
| | atggatgggcaaatggcgtggcgaaaaagtggcggtgaaagtattctttaccactga |
| | agaagccagctggtttcgagaaacagaaatctaccaaactgtgctaatgcgccatga |
| | aaacatacttggtttcatagcggcagacattaaaggtacaggttcctggactcagct |
| | ctatttgattactgattaccatgaaaatggatctctctatgacttcctgaaatgtgc |
| | tacactggacaccagagccctgcttaaattggcttattcagctgcctgtggtctgtg |
| | ccacctgcacacagaaatttatggcacccaaggaaagcccgcaattgctcatcgaga |
| | cctaaagagcaaaaacatcctcatcaagaaaaatgggagttgctgcattgctgacct |
| | gggccttgctgttaaattcaacagtgacacaaatgaagttgatgtgcccttgaatac |
| | cagggtgggcaccaaacgctacatggctcccgaagtgctggacgaaagcctgaacaa |
| | aaaccacttccagccctacatcatggctgacatctacagcttcggcctaatcatttg |
| | ggagatggctcgtcgttgtatcacaggagggatcgtggaagaataccaattgccata |
| | ttacaacatggtaccgagtgatccgtcatacgaagatatgcgtgaggttgtgtgtgt |
| | caaacgtttgcggccaattgtgtctaatcggtggaacagtgatgaatgtctacgagc |
| | agttttgaagctaatgtcagaatgctgggcccacaatccagcctccagactcacagc |
| | attgagaattaagaagacgcttgccaagatggttgaatcccaagatgtaaaaatctg |
| | atggttaaaccatcggaggagaaactctagactgcaagaactgtttttacccatggc |
| | atgggtggaattagagtggaataaggatgttaacttggttctcagactctttcttca |
| | ctacgtgttcacaggctgctaatattaaacctttcagtactcttattaggatacaag |
| | ctgggaacttctaaacacttcattctttatatatggacagctttattttaaatgtgg |
| | tttttgatgcctttttttaagtgggtttttatgaactgcatcaagacttcaatcctg |
| | attagtgtctccagtcaagctctgggtactgaattgcctgttcataaaacggtgctt |
| | tctgtgaaagccttaagaagataaatgagcgcagcagagatggagaaatagactttg |
| | ccttttacctgagactttcagttcgtttgtattctacctttgtaaaacagcctatag |
| | atgatgatgtgtttgggatactgcttattttatgatagtttgtcctgtgtccttagt |
| | gatgtgtgtgtgtctccatgcacatgcacgccgggattcctctgctgccatttgaat |
| | tagaagaaaataatttatatgcatgcacaggaagatattggtggccggtggttttgt |
| | gctttaaaaatgcaatatctgaccaagattcgccaatctcatacaagccatttactt |
| | tgcaagtgagatagcttccccaccagctttattttttaacatgaaagctgatgccaa |
| | ggccaaaagaagtttaaagcatctgtaaatttggactgttttccttcaaccaccatt |
| | ttttttgtggttattatttttgtcacggaaagcatcctctccaaagttggagcttct |
| | attgccatgaaccatgcttacaaagaaagcacttcttattgaagtgaattcctgcat |
| | ttgatagcaatgtaagtgcctataaccatgttctatattctttattctcagtaactt |
| | ttaaaagggaagttatttatattttgtgtataatgtgctttatttgcaaatcaccca |
| | ctcctttacaaccatactttatatatgtacatacattcatactgtagaaaccagctc |
| | atgtgtacctcatatcccatccttaagagaagaaatgttataaagtagaactaaata |
| | taaattttcagaattaatgcattcaaagtaatatatcaaatccaggactttgttaac |
| | ttcaggtaaaaacttcattagggtaatatcatctcaattttttcaaatgaaaggatt |
| | ctctaattagaaatttatatgtcagagctgttataaatttatcaactgtcaaatatg |
| | ttctggacagctaaatcatttgagatttttggttttttgatttctattccctaactt |
| | gtgaagacaatgaaaaatcaggcagaaatatttagtatctagtcagtatctgtagct |
| | acactgtataactgttcttcaataaaatggttcatattttatagatgccttgttatc |
| | tcaagaaatctgatttacataaacttatacttctttaatgctttttaaatatttatt |
| | ctgagcaaacaattcatgagtacatcaagtgagatagttttatttgattataacata |
| | aaataaatgtgattatatcacatcatcatcaaaaaggtttaaattaaatgggaggaa |
| | atcagcatatgtccacccattaccaaaatttgactatcatttaaggttaaaacttac |
| | aaatttgtctgcacatcaaatttcacaaatttgaaaattgccttaaccattttgatt |
| | aataagtttcatctgccataattaaagtctgaagtgttcatcaagataagtaaattt |
| | gcatatggataatacccaataacttgttttttcagaatttttcaccatatgtatact |
| | gagaaatacaaatattttaatctgcgttgccgtatgatatgattgcacttagaacac |
| | ccaatttacttaaatcttggtttacttttgacttgataccataatctttaaaatcat |
| | ttgtcatctttttttttttttttttgagacggagtctcgctctgtcgcccaggctgg |
| | actgcggactgcagtggcgcaatctcggctcactgcaagctccgcttcccgggttca |
| | cgccattctcctgcctcagcctcccgagtagctgggactacaggcgcccgccaccgc |
| | gcccggctaattttttgtatttttagtagagacggggtttcaccttgttagccagga |
| | tggtctcaatctcctgacctcatgatccacctgcctcggcctcccaaagttcatttg |
| | tcatcttaataaaaatataaagacaggcaaagtttattggaaatgttcaaatggtgt |
| | gtggaagcaaaaaattacagccagtatatgagaccactattatggttttttaaaatt |
| | aacttggtctagtaaaagtgatatcaagagttaatcttagaaacttgctcagtaaaa |
| | acattttctagtataacatgttctttaaaaagcaaatgctgccgtctttggaatctt |
| | aatctaaaaatgtggccgggcgcggtggctcacgcctgtaatcccaacactttggga |
| | ggctgaggcgggtggatcacaaggtcaggagttcaagaccagcctggccaacatggt |
| | gaaaccccatctctactaaaaataaaaaactcagccaggcgtggtggcgggtgcctg |
| | taatcccagctactcgggaggctgaagcaggagaattgcttaaaatcagaaggtgga |
| | ggttgcagtgagctgagatcgtgtcactgcactccagcctgggcaaaagagcgaaac |
| | tccatctcaaataaacaaacaaataaataacaaaaaacaaaaatgttgcattaaact |
| | tagttcttgtctctcctttccactcttattcttaaatctgaagctcatcgactaagt |
| | gaaatatttaaagaatatgataggccagcaagaagaagtattatgtagtaccatagt |
| | tagtaaattcgtaaaaccttggaagccattatttggtcccacttgcaatttagtgtt |
| | tttgaagtgtgtagcttcattcagatagctctttaaataattaaaatataaaagcaa |
| | acaacccaaactacctgactataaacaggaaaagttaaccctcaaagagagttcttg |
| | tgaattctctttatgctggcaaatagctctaggattaaaggcacattagggtttcct |
| | tcagtttgtttattctaagcttttactgtgctttttactgaacaagtttctgatgta |
| | taaaacttgcatctgatttctttggaaatattttcacaaaagttattttaatcagta |
| | tttttacattgcctttccagtgtccagaagtgtttctaaacttagaaagtgacctat |
| | agttttttaaaattatgttttcctagaacgtgccaaattttgatttactctaacaat |
| | cagtacttttcttcagatgctttgttctgtttagaacaaaaatgcactatagttttt |
| | aaagaatcatgcatctttgggttggcccaggatcaaatttgatattgaataatttat |
| | tccagggcagctttcataaacatacttcatagatgttgttttgaaatgtttctaaat |
| | atctaaaatcatttcaacagcagaaatgatttttattttaacaaaagattatgatag |
| | cccttgtagtgtttaaaagtggtcatatttattactgactttgagtcaggtgttaaa |
| | atagcagtgccacagctcgtctcttgccttagtgtgctgctgtgagagtcacagtgg |
| | aaactgcagggaggaggtgtgttcctaagaaccaaaatccagcacagcatcctgtga |
| | agccacgtgtaatgatggtcccataaggaaagtatgtgaatatggctcttgtaaagg |
| | attaactattgtaattttagcttatgctctgtattctgttttctatggaattattta |
| | agcccttttagtgacctttgtcctggcccatttaaaaactaaaatgtagtatatatt |
| | gtataaaatggaaatatcattattgcttcattaggggaaactgtacataggcattga |
| | aagaagggtaaaagcaagcagttttatcaggcagttgtaaaacaccaaaaatataga |
| | ttcgtctttgacgtgtaacacactaaatgtattttgtacagcatctggtttaaaagg |
| | tgccttaagagtttaccattacttgctttgttctatatacagattatgtccaatgta |
| | tcattttgaagtaaataaccttattttagtata |
| Protein sequence | MTQLYTYIRLLGACLFIISHVQGQNLDSMLHGTGMKSDLDQKKPENGVTLAPEDTLP |
| of mouse | FLKCYCSGHCPDDAINNTCITNGHCFAIIEEDDQGETTLTSGCMKYEGSDFQCKDSP |
| Bmpr1a (SEQ ID | KAQLRRTIECCRTNLCNQYLQPTLPPVVIGPFFDGSIRWLVVLISMAVCIVAMIIFS |
| NO: 3) | SCFCYKHYCKSISSRGRYNRDLEQDEAFIPVGESLKDLIDQSQSSGSGSGLPLLVQR |
| NCBI Reference | TIAKQIQMVRQVGKGRYGEVWMGKWRGEKVAVKVFFTTEEASWFRETEIYQTVLMRH |
| Sequence: | ENILGFIAADIKGTGSWTQLYLITDYHENGSLYDFLKCATLDTRALLKLAYSAACGL |
| NP_033888.2 | CHLHTEIYGTQGKPAIAHRDLKSKNILIKKNGSCCIADLGLAVKFNSDTNEVDIPLN |
| | TRVGTKRYMAPEVLDESLNKNHFQPYIMADIYSFGLIIWEMARRCITGGIVEEYQLP |
| | YYNMVPSDPSYEDMREVVCVKRLRPIVSNRWNSDECLRAVLKLMSECWAHNPASRLT |
| | ALRIKKTLAKMVESQDVKI |
| Nucleic acid | ccgcgcgagacgacgactgtacggccgcgcgaggggcgaccgggcccgggccgctgc |
| sequence | acgccgagggcggaggccgagccgggccccgccgccccgcggctgtccgtgcccgcc |
| encoding mouse | cgcgccgagcgccggaggatgagtttctcgggatcccgatttatgaaaatatgcatc |
| Bmpr1a (SEQ ID | gctttgatactgtcttggaattcatgagatggaagcataggtcaaagctgttcggag |
| NO: 4) | aaattggaactacagttttatctagccacatctctgagaattctgaagaaagcagca |
| NCBI Reference | ggtgaaagtcattgccaagtgattttgttctgtaaggaagcctccctcattcactta |
| Sequence: | caccagtgagacagcaggaccagtcattcaaagggccgtgtacaggacgcgtgcgaa |
| NM_009758.4 | tcagacaatgactcagctatacacttacatcagattactgggagcctgtctgttcat |
| | catttctcatgttcaagggcagaatctagatagtatgctccatggcactggtatgaa |
| | atcagacttggaccagaagaagccagaaaatggagtgactttagcaccagaggatac |
| | cttgcctttcttaaagtgctattgctcaggacactgcccagatgatgctattaataa |
| | cacatgcataactaatggccattgctttgccattatagaagaagatgatcagggaga |
| | aaccacattaacttctgggtgtatgaagtatgaaggctctgattttcaatgcaagga |
| | ttcaccgaaagcccagctacgcaggacaatagaatgttgtcggaccaatttgtgcaa |
| | ccagtatttgcagcctacactgccccctgttgttataggtccgttctttgatggcag |
| | catccgatggctggttgtgctcatttccatggctgtctgtatagttgctatgatcat |
| | cttctccagctgcttttgctataagcattattgtaagagtatctcaagcaggggtcg |
| | ttacaaccgtgatttggaacaggatgaagcatttattccagtaggagaatcattgaa |
| | agacctgattgaccagtcccaaagctctgggagtggatctggattgcctttattggt |
| | tcagcgaactattgccaaacagattcagatggttcggcaggttggtaaaggccgcta |
| | tggagaagtatggatgggtaaatggcgtggtgaaaaagtggctgtcaaagtgttttt |
| | taccactgaagaagctagctggtttagagaaacagaaatctaccagacggtgttaat |
| | gcgtcatgaaaatatacttggttttatagctgcagacattaaaggcactggttcctg |
| | gactcagctgtatttgattactgattaccatgaaaatggatctctctatgacttcct |
| | gaaatgtgccacactagacaccagagccctactcaagttagcttattctgctgcttg |
| | tggtctgtgccacctccacacagaaatttatggtacccaagggaagcctgcaattgc |
| | tcatcgagacctgaagagcaaaaacatccttattaagaaaaatggaagttgctgtat |
| | tgctgacctgggcctagctgttaaattcaacagtgatacaaatgaagttgacatacc |
| | cttgaataccagggtgggcaccaagcggtacatggctccagaagtgctggatgaaag |
| | cctgaataaaaaccatttccagccctacatcatggctgacatctatagctttggttt |
| | gatcatttgggaaatggctcgtcgttgtattacaggaggaatcgtggaggaatatca |
| | attaccatattacaacatggtgcccagtgacccatcctatgaggacatgcgtgaggt |
| | tgtgtgtgtgaaacgcttgcggccaatcgtgtctaaccgctggaacagcgatgaatg |
| | tcttcgagcagttttgaagctaatgtcagaatgttgggcccataatccagcctccag |
| | actcacagctttgagaatcaagaagacacttgcaaaaatggttgaatcccaggatgt |
| | aaagatttgacaattaaacaattttgagggagaatttagactgcaagaacttcttca |
| | cccaaggaatgggtgggattagcatggaataggatgttgacttggtttccagactcc |
| | ttcctctacatcttcacaggctgctaacagtaaaccttaccgtactctacagaatac |
| | aagattggaacttggaacttcaaacatgtcattctttatatatggacagctttgttt |
| | taaatgtggggtttttttgttttgctttttttgttttgttttggttttgatgctttt |
| | ttggtttttatgaactgcatcaagactccaatcctgataagaagtctctggtcaacc |
| | tctgggtactcactatcctgtccataaagtggtgctttctgtgaaagccttaagaaa |
| | attaatgagctcagcagagatggaaaaaggcatatttgccttctaccagagaaaaca |
| | tctgtctgtgttctgtctttgtaaacagcctatagattatgatctctttgggatact |
| | gcctggtttatgatggtgcaccatacctttgatatgcataccagaattctctgctgc |
| | cctagggcttagaagacaagaatgtaaaggttgcacaggaaggtatttgtggccagt |
| | ggtttaaatatgcaatatctagttgacaatcgccaatttcataaaagccatccacct |
| | tgtaactgtagtaacttctccactgactttatttttagcataatagttgtgaaggcc |
| | aaactccatgtaaagtgtccatagacttggactgttttcccccagtcaccattttgt |
| | tctccttttggtaattatttttgttataaaaagccacctatccagaattggagctct |
| | ctgtcttgaaccatactttgaaagaaacgcctcttccgtactgcatctgatcacaat |
| | gtgcatacctatgatcaaattctggagtctttgttctcggtacctcctaaaaaggaa |
| | agttgattcttgtgtaacatgcttttattttcagaacctgcacagctgtcattctag |
| | ccatgttttacctacacactcagttctatacaagacagcccatacactctgtctcac |
| | atctgatccttggtgggaagtgttttaaagtagaactatgtatgaatttcagaattc |
| | atgcattttaaaacttcactaagatattgtctcatatctttatgagaatgtcagctg |
| | acttttcaactaacagtaaatgtattttagatatctaaatcttttgaaatttggttt |
| | tacaatttctggtccctaattgtgaagacaagaggcagaagtacccagtcactaccc |
| | atatttacactgaacgttattaaataaaatgatgtgtattttattataaaataaata |
| | taggccttgttatctcaaaaaacagatctggttcaaacttattataccaatatcata |
| | ctatttaaatgttctaagtaaacaagccatgtgagcatcaagtggcattggctcttt |
| | ggatgaaacataaacttaaggtgattgtatcaacacatagagtgactgaaattaaat |
| | gggaggcaggtagagcatatgtccatctgtccacctacaggcatgactaaactacag |
| | ctcatattccacaaatttgagatttgtcttgcctggtttgtttagtgagtctcatct |
| | gatgtacctaaagcctgagagtactgaggtctgattttatatctttcctgaataaac |
| | taaatcttttttgtcacttatcatcttaatgatatacctaaggaataattctttggc |
| | atgtttcagttgtgtgtggcagccactgtaatgactcttctctaagaaaggctgtca |
| | ggagttaattataaggcaggcagtgagcgctctagtcactgccttcccacgctgcca |
| | tcactgcattcatgggaatcagtgacgttctcgaaatggcaaacgctgctgcttttc |
| | cttatttggaatcctaaaatcaaaagttgcattaaacttactgtgttctcttctccc |
| | tctcagccataaatgtaaaattcagtaagtaaaaatatttaaagagtgtatcagccc |
| | tttggccagtgagatagctcagtagataaaggcatttgctgccaagtcctcaacctc |
| | aattcagactctgggggacacatggcgaagggaggagccaactaccccatcattgtc |
| | ctctgacttccacacactccatggcttgcgcccctccccacagacacacaccatgta |
| | ctccacaaaagtagtttaaaggaaaaaaagaatagaacccactgtgtaatggaataa |
| | gtattatgtagttacttaacaacttgtaaaaatctggaaactatgatttggttcccc |
| | tttgaatctagagtttaaaaaacagatggctaaaatcagccatcatttaaataatta |
| | aaaataaaagccccaaaccccaaactgcctaaataaataccaagtaatccaggaagc |
| | cgtcatgtgtggtttgtatgaccagtagttctctggtacagagcatgttaagatttg |
| | ccccagcctgattctctgaggtctctgcattactgagtactgttctgagtataaaat |
| | ctgaactgatttctctagaaatactgtaacaaaaaggtattttagtcagcatgttat |
| | gttaacccttccactgtctagaaacttgaataagcacataaagacaccttttgctgt |
| | catcatctgttgtcctggaatgtgccagttttaatttattcattctaatgatattca |
| | atttgcttttcttttttagatgtttttcttgtttagagtaaaaggacgaatttttca |
| | agaaccttgcatctctgatttggcctaaggtcaaattggatattgagtagtctattc |
| | cagggcagatttcctaagcaatacttgtcttttcagctatgtattgttttgaaatgt |
| | ttccatttcaacagaggtgtaagtcatgtgaaaagaaaggtggtgtagcccttgtgg |
| | taatgacacaagttgacttgcgtcagatgttaagcagggacagttctcccacctcct |
| | ggctgtaaggagtggaaactaggcaagcagtgtatcagtccacagaggacaggaagg |
| | gtcatcccataaagaaagcctgtgagtatggctttggcaaaaaattagacataatac |
| | tgtccttttaggttgtgctctgttctttcctttcagtggaattatttaagctcttta |
| | gtggcctttgtttttcccacttaaaaactaaaatgtagcatatattgtataaaatgg |
| | aaatattaatagcttagggaaactgtacataaggcattgacaggtttaaaaaaagca |
| | tttttattatgcagttgtaaaacaccaaaaatatagattcatcttgatatgtaacac |
| | taagtgtattttgtacagcatctgatttgaaaggtgccttatgaagtttaccattaa |
| | ttgctttgttctatatacagattatgtccaatgtatcatttttcagtaaataacctt |
| | attttagta |

More significantly, it was found that BMPR1A is a cell surface marker of human SuSCs. Using an ex vivo system, the inventors showed that SuSCs maintained sternness properties for an extended period without losing the osteogenic ability. This study advances the knowledge base of congenital deformity and regenerative medicine mediated by skeletal stem cells. In addition to BMPR1A, additional positive markers and negative markers are disclosed herein.

Examples of positive markers for SSCs include BMPR1A, Axin2, BMP2, BMP3, BMP4, BMP6, BMP7, BMP8b, BMP15, Gremlin1, CD200, AlphaV, cathepsin K, Gli1, Ltf, Camp, Ear1, Lcn2, Ngp, Chil3, Anxa1, Prg2, Elane, and Alox15. Listed in Table 1 below are additional examples of positive markers. Examples of positive markers for SSCs include CD31, CD45, Ter-119, Tie 2, Thy, and CD105. Listed in Table 2 below are additional examples of negative markers. The markers and their respective homologues can be used to identify, enrich, or isolate skeletal stem cells and suture stem cells of an animal subject, such as human or mouse.

TABLE 1
Positive/Upregulated genes in SSC

| Gene.Symbol | mRNA.Accession |
|---|---|
| Ltf | NM_008522 |
| Chi3l3 | NM_009892 |
| Anxa1 | NM_010730 |
| 1100001G20Rik | NM_183249 |
| Elane | NM_015779 |
| Alox15 | NM_009660 |
| Cebpe | NM_207131 |
| C3 | NM_009778 |
| Hp | NM_017370 |
| Cnnm2 | NM_033569 |
| Lrg1 | NM_029796 |
| Serpinb2 | NM_001174170 |
| Pygl | NM_133198 |
| Gca | NM_145523 |
| Abhd5 | NM_026179 |
| Cd63 | NM_001042580 |
| Cd177 | NM_026862 |
| Slfn4 | NM_011410 |
| Chi3l1 | NM_007695 |
| Prtn3 | NM_011178 |
| Itgb2l | NM_008405 |
| Mpo | NM_010824 |
| Fam101b | NM_029658 |
| Col3a1 | NM_009930 |
| Dstn | NM_019771 |
| Gsr | NM_010344 |
| Olr1 | NM_138648 |
| Thbs1 | NM_011580 |
| Pilrb1 | NM_133209 |
| Vasp | NM_009499 |
| Ibsp | NM_008318 |
| Gadd45a | NM_007836 |
| Ankrd22 | NM_024204 |
| Anxa3 | NM_013470 |
| Col1a2 | NM_007743 |
| Mmp25 | NM_001033339 |
| Tkt | NM_009388 |
| G6pdx | NM_008062 |
| Padi4 | NM_011061 |
| Alas1 | NM_020559 |
| Dach1 | NM_007826 |
| Kcnj14 | NM_145963 |
| Cd300lf | NM_001169153 |
| Col1a1 | NM_007742 |
| Mrgpra2a | NM_001172588 |
| Pnkp | NM_021549 |
| Actn1 | NM_134156 |
| Plbd1 | NM_025806 |
| Pgd | NM_001081274 |
| Gpi1 | NM_008155 |

TABLE 2
Negative/Downregulated genes in SSC

| Gene.Symbol | mRNA.Accession |
|---|---|
| Spib | NM_019866 |
| Vpreb3 | NM_009514 |
| Cd74 | NM_001042605 |
| H2-Aa | NM_010378 |
| Blnk | NM_008528 |
| Cd72 | NM_001110320 |
| Cecr2 | NM_001128151 |
| Cd79a | NM_007655 |
| Vpreb1 | NM_016982 |
| Irf4 | NM_013674 |
| Tspan13 | NM_025359 |
| Ly6d | NM_010742 |
| Ikzf3 | NM_011771 |
| Rag1 | NM_009019 |
| H2-Ab1 | NM_207105 |
| Pyhin1 | NM_175026 |
| Scd1 | NM_009127 |
| Bcl7a | NM_029850 |
| Gpr171 | NM_173398 |
| Pou2af1 | NM_011136 |
| Cpm | NM_027468 |
| Il7r | NM_008372 |
| Cd2 | NM_013486 |
| H2-Eb1 | NM_010382 |
| Myl4 | NM_010858 |
| Fam129c | NM_001166213 |
| Il12a | NM_001159424 |
| Dpp4 | NM_010074 |
| Atp1b1 | NM_009721 |
| Serinc5 | NM_172588 |
| Slc1a5 | NM_009201 |
| Smarca4 | NM_001174078 |
| H2-Q6 | NM_207648 |
| Klhl24 | NM_029436 |
| Zfp706 | NM_026521 |
| Glcci1 | NM_133236 |
| Crip1 | NM_007763 |
| Fcrla | NM_001160215 |
| Cd69 | NM_001033122 |
| Cytip | NM_139200 |
| Cnp | NM_009923 |
| S1pr1 | NM_007901 |
| Ccl5 | NM_013653 |
| Strbp | NM_009261 |
| Slamf7 | NM_144539 |
| Igll1 | NM_001190325 |
| Ms4a1 | NM_007641 |
| Zfp36l1 | NM_007564 |
| Cd93 | NM_010740 |
| Nfkbie | NM_008690 |

### Identification, Isolation, and Enrichment of SSCs

In one aspect, the present application relates to identification, isolation and enrichment of SSCs and in particular SuSCs. For example, these cells can be separated from a complex mixture of cells by techniques that identify or enrich for cells expressing BMPR1A alone or in combination with other markers and characteristics as described herein.

Mammalian SSCs, such as mouse SSCs and human SSCs may be characterized with the functional homologs of BMPR1A, and optionally other markers, including CD200, 6C3, PDPN, CD105, CD90, CD45, Tie2, αv integrin, and others disclosed herein. Methods and compositions are provided for the separation and characterization of SSCs and bone progenitor cells. The cells may be separated from other cells by the expression of these specific cell surface markers.

Cells of interest, i.e. cells expressing a marker of choice, may be isolated or enriched for, that is, separated from the rest of the cell population, by a number of methods that are well known in the art. For example, flow cytometry, e.g. fluorescence activated cell sorting (FACS), may be used to separate the cell population based on the intrinsic fluorescence of the marker, or the binding of the marker to a specific fluorescent reagent, e.g. a fluorophor-conjugated antibody, as well as other parameters such as cell size and light scatter. In other words, the selection of the cells may be achieved by flow cytometry. Although the absolute level of staining may differ with a particular fluorochrome and reagent preparation, the data can be normalized to control. To normalize the distribution to control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype-matched control, but are not as intense as the most brightly staining cells normally found in the population. An alternative control may utilize a substrate having a defined density of marker on its surface, for example, a fabricated bead or cell line, which provides the positive control for intensity. Other methods of separation, i.e. methods by which selection of cells may be achieved, based upon markers include, for example, magnetic activated cell sorting (MACS), immunopanning, and laser capture microdissection.

Populations that are enriched by selecting for the expression of one or more markers will usually have at least about 80% cells of the selected phenotype, more usually at least 90% cells and can be 95% of the cells, or more, of the selected phenotype.

In some examples, for isolation of cells from a tissue, an appropriate solution may be used for dispersion or suspension. Such solution generally can be a balanced salt solution, e.g. normal saline, PBS, Hanks balanced salt solution, etc., supplemented with fetal calf serum or other naturally occurring factors, in conjunction with an acceptable buffer at low concentration, generally from 5-25 mM. Convenient buffers include HEPES, phosphate buffers, lactate buffers, etc. The tissue may be enzymatically and/or mechanically dissociated. In some embodiments, bone tissue is treated with a gentle protease, e.g. dispase, etc., for a period of time sufficient to dissociate the cells, then is gently mechanically dissociated.

An initial separation may select for cells by various methods known in the art, including elutriation, Ficoll-Hypaque or flow cytometry using the parameters of forward and obtuse scatter. Separation of the SSC population will then use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, 7-AAD). Any technique may be employed which is not unduly detrimental to the viability of the selected cells.

The affinity reagents may be specific receptors or ligands for the cell surface molecules indicated above. Of particular interest is the use of antibodies as affinity reagents. The details of the preparation of antibodies and their suitability for use as specific binding members are well known to those skilled in the art. Depending on the specific population of cells to be selected, antibodies having specificity for BMPRIA can be contacted with the starting population of cells. Optionally, reagents specific for one or more of the other markers disclosed herein can also be included.

As is known in the art, the antibodies can be selected to have specificity for the relevant species, i.e. antibodies specific for human markers are used for the selection of human cells; antibodies specific for mouse markers are used in the selection of mouse cells, and the like.

These antibodies can be conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Examples of fluorochromes that can be used include phycobiliproteins, e.g. phycoerythrin and allophycocyanins, fluorescein and Texas red. Frequently each antibody is labeled with a different fluorochrome, to permit independent sorting for each marker.

The antibodies can be added to a suspension of cells, and incubated for a period of time sufficient to bind the available cell surface antigens. The incubation usually can be at least about 5 minutes and usually less than about 2 hours. It is desirable to have a sufficient concentration of antibodies in the reaction mixture, such that the efficiency of the separation is not limited by lack of antibody. The appropriate concentration can be determined by titration. The medium in which the cells are separated can be any medium that maintains the viability of the cells. An exemplary medium can be phosphate buffered saline containing from about 0.1 to 20% BSA or FBS. Various media are commercially available and may be used according to the nature of the cells, including those described above, such as Dulbeccos Modified Eagle Medium (DMEM), Hank's Basic Salt Solution (HBSS), Dulbeccos phosphate buffered saline (dPBS), RPMI, Iscoves medium, PBS with 5 mM EDTA, etc., frequently supplemented with fetal calf serum, BSA, HSA, etc.

The labeled cells are then separated as to the phenotype described above. The separated cells may be collected in any appropriate medium that maintains the viability of the cells, usually having a cushion of serum at the bottom of the collection tube. Various media are commercially available and may be used according to the nature of the cells, including DMEM, HBSS, dPBS, RPMI, Iscoves medium, etc., frequently supplemented with fetal calf serum.

Compositions highly enriched for SSC can be achieved in this manner. The cell population can contain about 50% or more of the SSCs, and usually at or about 90% or more of SSCs, and can be as much as about 95% or more of SSCs. The enriched cell population may be used immediately, or be frozen at liquid nitrogen temperatures and stored for long periods of time, being thawed and capable of being reused. The cells can usually be stored in 10% DMSO, 50% FCS, 40% medium (e.g. RPMI 1640 medium). Once thawed, the cells may be expanded by the use of growth factors cells for proliferation and differentiation.

In some embodiments, the cells are identified, isolated, or enriched from a tissue. The tissue may be from any animal of interest. Examples include vertebrate animals, such as mammals and non-mammals, e.g., fishes, amphibians, and birds, which have similar skeletal structures and skeletal stem cells (Mork and Crump, Curr Top Dev Biol, 2015). Examples of mammals include humans, primates, domestic and farm animals, and zoo, laboratory or pet animals, such as dogs, cats, cattle, horses, sheep, pigs, goats, rabbits, rats, mice, etc. The cells may be established cell lines or they may be primary cells, where “primary cells”, “primary cell lines”, and “primary cultures” are used interchangeably herein to refer to cells and cells cultures that have been derived from a subject and allowed to grow in vitro for a limited number of passages.

The SSCs may be isolated from fresh or frozen cells, which may be from a neonate, a juvenile or an adult, and from tissues including skin, muscle, bone marrow, peripheral blood, umbilical cord blood, spleen, liver, pancreas, lung, intestine, stomach, adipose, and other tissues. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution may be used for dispersion or suspension. Such solution will generally be a balanced salt solution, e.g. normal saline, PBS, Hank's balanced salt solution, etc., supplemented with fetal calf serum or other naturally occurring factors, in conjunction with an acceptable buffer at low concentration, generally from 5-25 mM. Convenient buffers include HEPES, phosphate buffers, lactate buffers, etc.

### Cell Culturing, Expanding, and Differentiating

The SSCs described above may be cultured and maintained in vitro under various culture conditions. A suitable culture or nutrient medium may be liquid or semi-solid (e.g. containing agar, methylcellulose, etc.). The cell population may be suspended in an appropriate nutrient medium, such as DMEM, Iscove's modified DMEM or RPMI-1640, normally supplemented with fetal calf serum (about 1-25%), L-glutamine, a thiol, particularly 2-mercaptoethanol, and antibiotics. In one embodiment of the invention, the SSCs can be maintained in culture in the absence of feeder layer cells or in the absence of serum, etc. Examples of a culture or nutrient medium include DMEM, DMEM/F12, DMEM high glucose, MEM, IMDM, Gibco StemPro™-34 SFM, Gibco Essential 8, Gibco Essential 6, Gibco MesenPRO, Gibco StemPro MSC, Gibco CTS KnockOut SR XenoFree, Corning NutriStem hPSC XF, and HyClone AdvanceSTEM.

The culture may contain antibiotics to prevent the growth of bacteria and fungi include. Examples of antibiotics include penicillin, streptomycin, Amphotericin B, Gentamicin, Puromycin, Hygromycin B, Ciprofloxacin, Chloramphenicol, Kanamycin, Neomycin, Blasticidin S, G418 Sulfate, Ampicillin, and Carbenicillin.

The culture or medium may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

The growth factor may be any suitable growth factor. Examples include bone morphogenic protein (BMP); Indian hedgehog (IHH); transforming growth factor 13 (TGF (3); bone morphogenetic proteins (BMPs) for example BMP-2, 4, 6 and 9; fibroblast growth factors (FGFs) like FGF-1 and 2; an epithelial cell growth factor (EGF); Wnt ligands and (3-catenin; insulin-growth factors (IGFs) like IGF-1 and IGF-2; Collagen-1; Runx2; Osteopontin; Osterix; vascular endothelial growth factor (VEGF); platelet derived growth factor (PDGF); osteoprotegerin (OPG); NEL-like protein 1 (NELL-1); or any combination thereof. For in detail disclosure of such growth factors, see, for example, Devescovi, V. et al. “Growth factors in bone repair” Chir Organi Mov 92, 161, 2008; James A. W. “Review of Signaling Pathways Governing MSC Osteogenic and Adipogenic Differentiation” Scientifica (Cairo) 2013; 2013: 684736; Carofino B. C. et al. “Gene therapy applications for fracture healing” J Bone Joint Surg Am, 90 (Suppl 1) (2008), pp. 99-110; Javed A. et al. “Genetic and transcriptional control of bone formation” Oral Maxillofac Surg Clin North Am. 2010; 22: 283-93; Chen G. et al. “TGF-13 and BMP signaling in osteoblast differentiation and bone formation” Int. J. Biol. Sci. 2012; 8:272-288; Ornitz, D. M. et al. “FGF signaling pathways in endochondral and intramembranous bone development and human genetic disease. Genes Dev. 16, 1446-1465 (2002); Krishnan V. et al. “Regulation of bone mass by Wnt signaling” J. Clin. Invest. 116 1202-1209 (2006); and Boyce B. F. et al. Arch Biochem Biophys. 473(2):139-146 (2008). The entire content of each of these publications is incorporated herein by reference.

The SSCs may be directed to differentiate along a specific path under conditions known in art. For example, SSCs can be directed to chondrogenesis by differentiation factors, which may be referred to herein as chondrogenesis factors. In some examples, the SSCs can be differentiated to a desired skeletal lineage cell. Specific embodiments include the skewing of differentiation from a skeletal stem cell to a chondrocyte by contacting with an effective dose of one or both of a VEGF inhibitor and a TGFβ inhibitor; and the use thereof in tissue repair. Other examples of skeletal cells that may be generated by the methods described herein pre-bone cartilage and stromal progenitor (pre-BCSP), BCSP, committed cartilage progenitor (CCP), bone progenitor, B-cell lymphocyte stromal progenitors (BLSP); 6C3 stroma, hepatic leukemia factor expressing stromal cell (HEC); and progeny thereof

Cells contacted in vitro with the factors, e.g., the factors that promote reprogramming and/or promote the growth and/or differentiation of chondrocytes, and the like, may be incubated in the presence of the reagent(s) for about 30 minutes to about 24 hours, which may be repeated with a frequency of about every day to about every 4 days, e.g., every 2 days, every 3 days. The agent(s) may be provided to the cells one or more times, and the cells allowed to incubate with the agent(s) for some amount of time following each contacting event e.g. 16-24 hours, after which time the media is replaced with fresh media and the cells are cultured further.

After contacting the cells with the factors, the contacted cells may be cultured so as to promote the survival and differentiation of skeletal stem cells, chondrocytes, or progenitor cell populations defined herein. Methods and reagents for culturing cells are known in the art, any of which may be used in here to grow and isolate the cells. For example, the cells (either pre- or post-contacting with the factors) may be plated on Matrigel or other substrates as known in the art. The cells may be cultured in media, supplemented with factors. Alternatively, the contacted cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by the use of growth factors and/or stromal cells associated with skeletal survival and differentiation.

Induced skeletal or chondrogenic cells produced by the above methods may be used in cell replacement or cell transplantation therapy to treat diseases. Specifically, the cells may be transferred to subjects suffering from a wide range of diseases or disorders with a skeletal or cartilaginous component.

In some cases, the cells or a sub-population of cells of interest may be purified or isolated or enriched from the rest of the cell culture prior to transferring to the subject. In some cases, one or more antibodies specific for a marker of cells of the skeletal/chondrogenic lineage or a marker of a sub-population of cells of the skeletal lineage are incubated with the cell population and those bound cells are isolated. In other cases, the cells or a sub-population of the cells express a marker that is a reporter gene, e.g. EGFP, dsRED, lacz, and the like, that is under the control of a specific promoter, which is then used to purify or isolate the cells or a subpopulation thereof

Spheres

In one aspect, the present application relates to a sphere of cells comprising one or more BMPR1A⁺ cells. The sphere can be 20 to 200 μm in diameter, or have about 10 to 500 (e.g., 50-400, 100-300, etc.) cells per sphere. The sphere can comprises Axin2+ cells. To make such spheres, one can seed a population of suture stem cells or skeletal stem cells in a maintaining medium described above on a low attachment surface or ultra-low attachment surface and culture the cells or the progenies thereof for a period of time to form one or more spheres. The period of time can be 5-20 days, such as 5-15 days and 7-10 days.

A low attachment surface or ultra-low attachment surface refers to a surface for cell culture, which is treated to reduce or minimize cell adherence. Such a surface can have a neutral, hydrophilic hydrogel coating that greatly reduces binding of attachment proteins. This minimizes cell attachment and spreading. A covalently bound hydrogel layer effectively inhibits cellular attachment and minimizes protein absorption, enzyme activation, and cellular activation. Cell culture with such ultra-low attachment surface are available from manufactures such as CORNING.

The terms “sphere” and “sphere-like cell aggregate” are used interchangeably to refer a cell aggregate having a stereoscopic shape close to globular. Examples of the stereoscopic shape close to globular include a globular shape which is a shape having a three-dimensional structure and indicating, when projected onto a two-dimensional surface, for example, a circle or an ellipse, and a shape formed by fusing a plurality of globular shapes (indicating, for example, when projected onto a two-dimensional surface, a shape formed by overlapping two to four circles or ellipses). The term “cell aggregate” refers to a ball-shaped cluster of cells or block of cells including pluripotent stem cells, such as SSCs. The cell aggregate may have a spherical shape. The cell aggregate may be a sphere. The sphere or aggregate is preferably formed by suspension culturing. The sphere or cell aggregate is a cluster of cells including undifferentiated pluripotent stem cells. The sphere or cell aggregate has a capability of producing various types of cells when the sphere/cell aggregate is cultured.

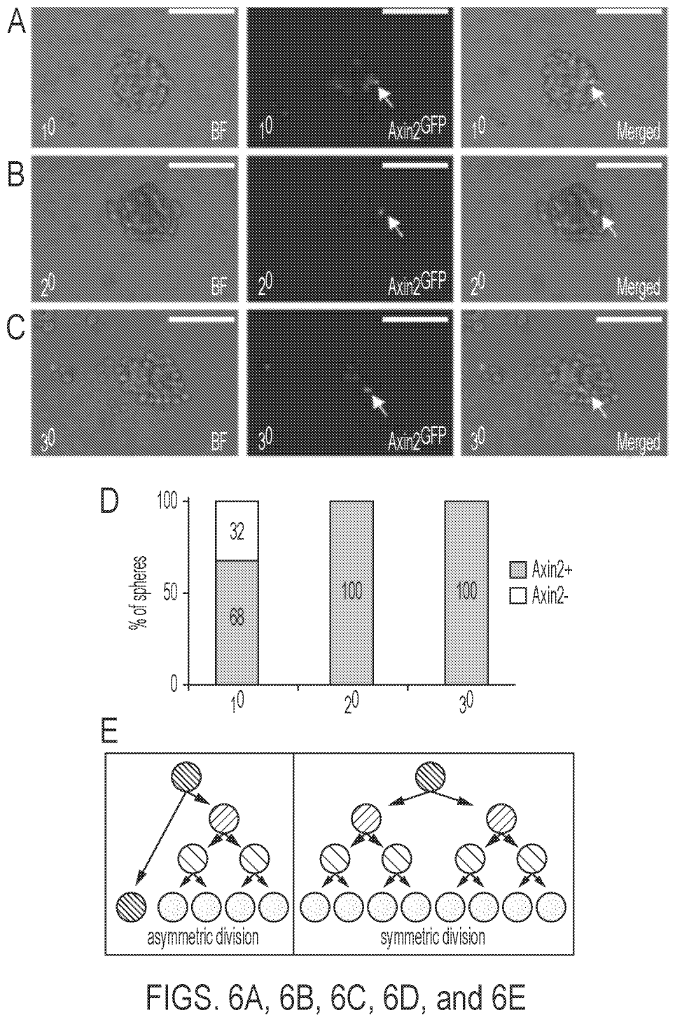

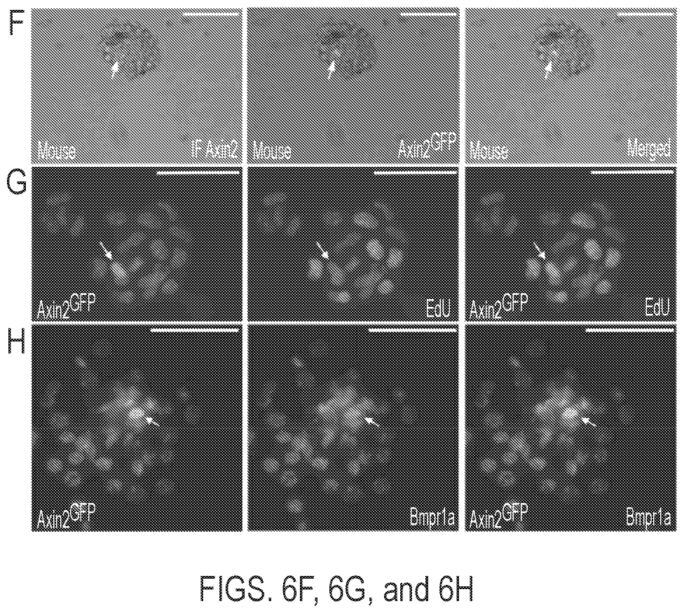

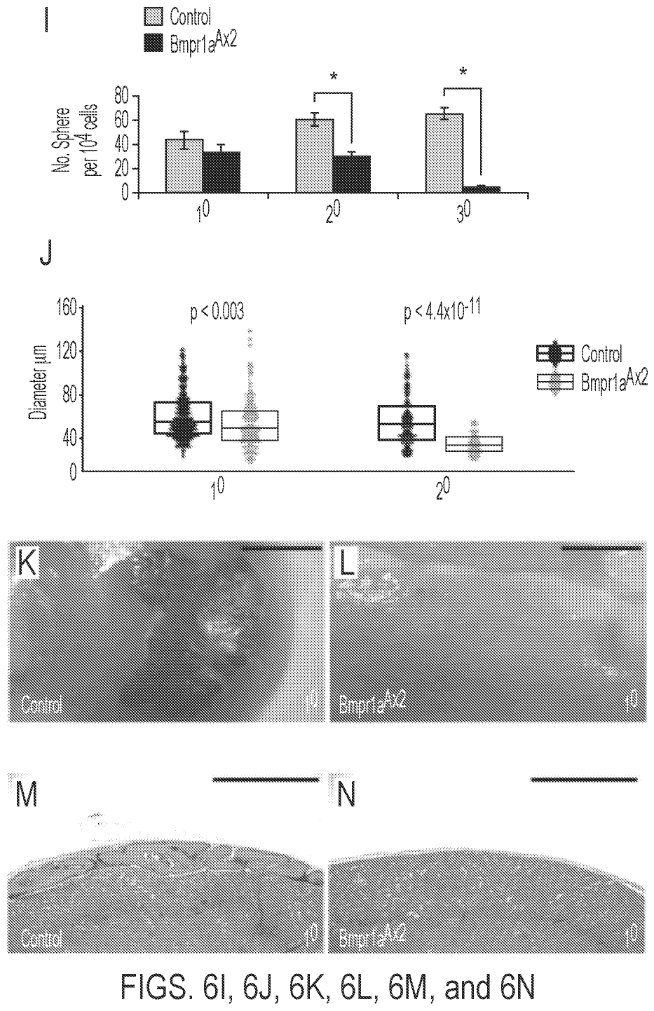

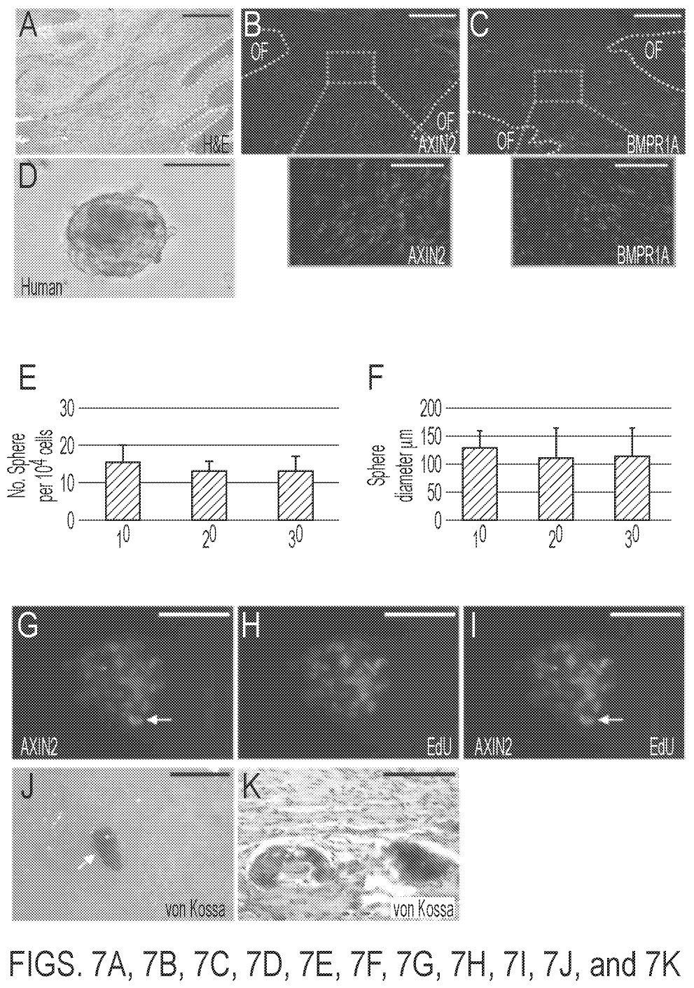

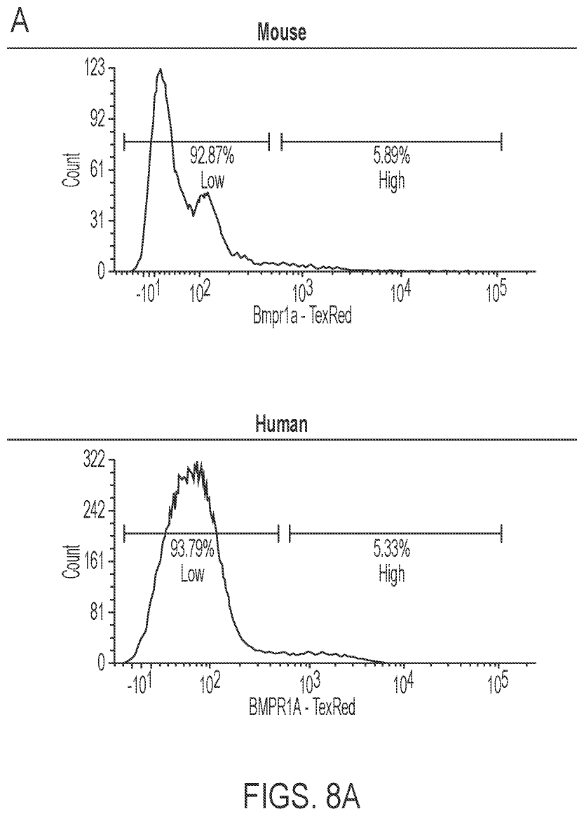

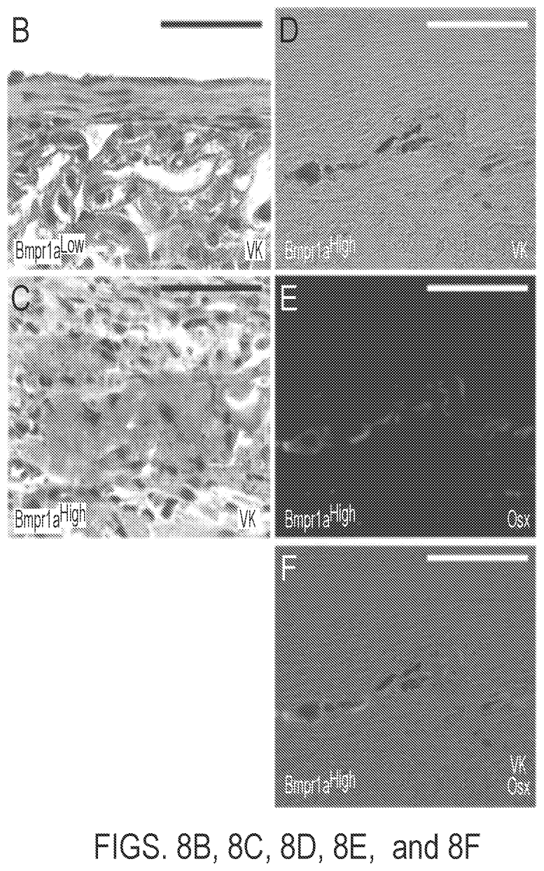

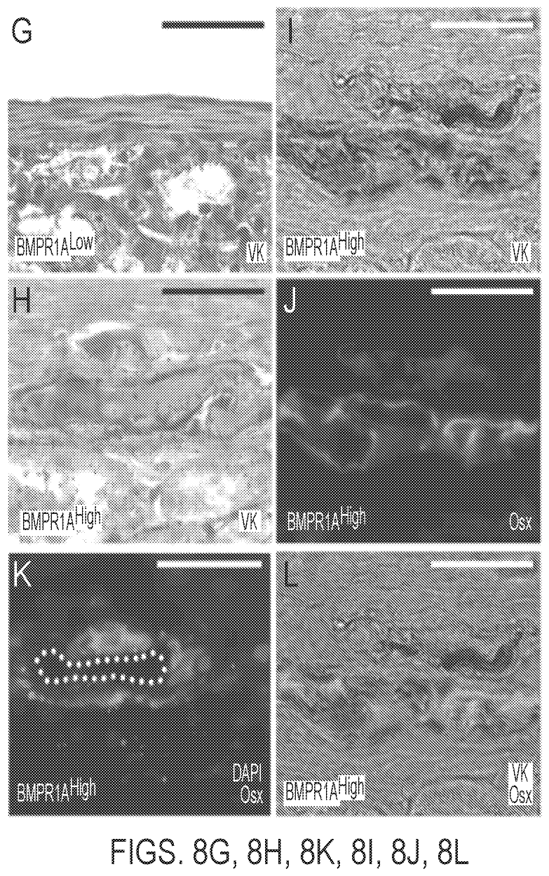

As disclosed in the examples below, about 5% of mouse suture mesenchymal cells can be Bmpr1A⁺ cells (FIG. 8A) and about 0.5% of suture mesenchymal cells may form a sphere (FIG. 6I, control). Most of the spheres include Bmpr1a+ cells (FIG. 6H). Therefore, approximately 10% of Bmpr1a+ cells form a sphere. For human stem cells, about 5% of suture mesenchymal cells are BMPR1A⁺ cells (FIG. 8A) and about 0.1% of suture mesenchymal cells form a sphere in current culture condition (FIG. 7E). Most of the spheres include BMPR1A⁺ cells. Therefore, about 2% of BMPR1A⁺ suture mesenchymal cells form a sphere.

As disclosed in the examples below, in primary sphere culture, there are 68% of spheres contain Axin2+ cells. In subsequent secondary and tertiary cultures, 100% of spheres contain Axin2+ cells. In theory, all spheres should be formed by Axin2+ cells as there are stem cells with “unlimited self-renewal” ability which require to form a sphere in sequential cultures for the true definition of the stem cell. An explanation for the primary culture is that some spheres (32%) are formed by progenitor cells with limited proliferation ability so can form sphere only in the primary culture but not secondary culture. Axin2+ stem cells are quiescent which gives rise to the sphere by asymmetric cell division. A sphere containing no Axin2+ cells means there is no Axin2+ stem cell with quiescent/slow-cycling features. See FIG. 6.

The cells, spheres, and methods described herein are useful in the development of an in vitro or in vivo model for bone function, for gene therapy, and for artificial organ construction. For example, the developing bones can serve as a source of growth factors and pharmaceuticals and for the production of viruses or vaccines, for in vitro toxicity and metabolism testing of drugs and industrial compounds, for gene therapy experimentation, for the construction of artificial transplantable bones, and for bone mutagenesis and carcinogenesis.

### Bone Regeneration Product

The cells described herein can be provided in combination with other types of cells, agents, materials, and structures for various uses, such as tissue engineering and treatment of any bone with defect. Accordingly, this application provides a bone regeneration product or bone regeneration formulation comprising at least one skeletal stem cell and optionally at least one of such other type of cells, agents, material, and structure. To that end, the cells can be in a three-dimensional (3D) synthetic, semi-synthetic, or living biological tissues.

In one example, the bone regeneration product/formulation comprises at least one SSC and at least one scaffold suitable for carrying the cell. The scaffold may comprise any 3D-printed scaffold suitable for carrying the cell. The bone regeneration product/formulation is suitable for a dense bone regeneration, a spongy bone regeneration, or a combination thereof. The bone regeneration product/formulation may further comprise a growth factor as mentioned above.

The scaffold can comprise various suitable materials, such as hydroxyapatite (HA) tricalcium phosphate (TCP), and a polymer. The polymer may be prepared by using photocurable polymers and/or monomers.

The scaffold may comprise a porous, 3D network of interconnected void spaces. The scaffold may be any scaffold suitable to incorporate the cells and/or growth factors disclosed herein to aid in forming a direct contact and/or an indirect contact of these cells and/or growth factors with a tissue (e.g. bone) for the regeneration of this tissue (e.g. bone). The scaffold may incorporate the cells and/or growth factors in any form, for example, by carrying, by supporting, by adsorbing, by absorbing, by encapsulating, by holding, and/or by adhering to the cells and/or growth factors.

The scaffold may have any shape or geometry. The scaffold may have any pore size. The scaffold may have any porosity (i.e. void volume.) The scaffold may have any form. The scaffold may have any mechanical strength.

Bone defects may form in different parts of an animal or a human body. These defects may have any shape and size. Scaffolds suitable for the treatment of such defects may have shapes, volumes and sizes that can, for example, fit to or resemble the defect shape and size. Such scaffolds may also have pore volumes, pore sizes, and/or pore shapes that resemble to the bone for which the bone regeneration products that comprise such scaffolds are designed for their treatment. Such scaffolds may also have pores with pore sizes sufficiently small such that these scaffolds can contain the cells described herein within their porous structures and allow the bone regeneration product to be implanted and the treatment can be successfully carried out. The bone regeneration products/formulation can have a mechanical strength sufficient enough to handle load bearing conditions of their implantation to a body. It can also have a mechanical strength sufficient enough to handle load bearing conditions of bones during motion (e.g. walking) and/or weight of the bodies.

The scaffold may comprise any material. For example, the scaffold may comprise a non-resorbable material, resorbable material, or a mixture thereof. The resorbable material may be resorbed by the body of a patient, and eventually replaced with healthy tissue. A “resorbable” material may comprise, for example, a biocompatible, bioabsorbable, biodegradable polymer, any similar material, or a mixture thereof.

A biocompatible material is a material that may be accepted by and to function of a body of a patient without causing a significant foreign body response (such as, for example, an immune, inflammatory, thrombogenic, or like response), and/or is a material that may not be clinically contraindicated for administration into a tissue or organ. The biodegradable material may comprise a material that is absorbable or degradable when administered in vivo and/or under in vitro conditions. Biodegradation may occur through the action of biological agents, either directly or indirectly.

The scaffold may comprise a solid, a liquid, or a mixture thereof. For example, the scaffold may be a paste. For example, the scaffold may comprise a paste comprising a mixture of hydroxyapatite and tricalcium phosphate (HA/TCP). This scaffold, for example, may be prepared by mixing hydroxyapatite (HA) and tricalcium phosphate (TCP) with a formulation comprising a liquid to prepare a paste. For example, the mesenchymal stem cell formulation may comprise a liquid; and mixing of such mesenchymal stem cell formulation with hydroxyapatite (HA) and tricalcium phosphate (TCP) may form a paste. This type of scaffold is called an HA/TCP scaffold herein.

A mixture comprising HA and TCP (HA/TCP) may be formed from equal amounts of HA and TCP in weight, for example 50 wt % HA and 50 wt % TCP, unless otherwise stated. However, the mixture comprising HA and TCP may have any composition, for example, varying in the range of 0 wt % HA to 100 wt % TCP. For example, an HA concentration higher than 10 wt %, higher than 20 wt %, higher than 30 wt %, higher than 40 wt %, higher than 50 wt %, higher than 60 wt %, higher than 70 wt %, higher than 80 wt %, or higher than 90 wt % is within the scope of this application.

The scaffolds described herein may comprise any biodegradable polymer. For example, the scaffold may comprise a synthetic polymer, naturally occurring polymer, or a mixture thereof. Examples of suitable biodegradable polymers may be polylactide (PLA), polyglycolide (PGA), poly(lactide-co-glycolide) (PLGA), poly-E-caprolactone, polydioxanone trimethylene carbonate, polyhybroxyalkonates (e.g., poly(hydroxybutyrate)), poly(ethyl glutamate), poly(DTH iminocarbony (bisphenol A iminocarbonate), poly(ortho ester), polycyanoacrylates, fibrin, casein, serum albumin, collagen, gelatin, lecithin, chitosan, alginate, poly-amino acids (such as polylysine), and a mixture thereof.

The scaffold may further comprise one or more of the growth factors described above. The growth factor may be continuously released to the surrounding (bone) tissue after the bone regeneration product is implanted into the bone defect site. The release rate of the growth factor may be controlled through the scaffolds' chemical composition and/or pore structure.

The scaffold may be manufactured by any technique. For example, the scaffold may be manufactured by hand, and/or by using a machine. For example, the scaffold may be manufactured by additive manufacturing and/or manufacturing. For example, the scaffold may be manufactured using a combination of more than one such manufacturing techniques.

In one embodiment, the cells are used in a “bio-printing” process to generate a spatially-controlled cell pattern using a 3D printing technology. Any bio-printing or bio-fabricating process known in the art can be used, e.g., as described in U.S. Pat. App. Pub. Nos. 20140099709, 20140093932, 20140274802, 20140012407, 20130345794, 20130190210 and 20130164339; and U.S. Pat. No. 8,691,974.

For example, in one embodiment, a printer cartridge is filled with a suspension of SSCs or SSC spheres and a gel. The alternating patterns of the gel and cells or spheres are printed using a standard print nozzle. In an alternative embodiment, a NOVOGEN (San Diego, Calif.) MMX™, or Organovo Holdings, Inc., bioprinters can be used for 3D bioprinting. This and equivalent “bio-printers” can be optimized to “print”, or fabricate, bone tissue, cartilage tissue, and other tissues, all of which are suitable for surgical therapy and transplantation.

Any 3D printing technique may be used to manufacture the scaffold. The 3D printing technique or the additive manufacturing (AM) may be a process for making a physical object from a 3D digital model, typically by laying down many successive thin layers of a material. Such thin layers of material may be formed under computer control. Examples of the 3D printing technologies may be Stereolithography (SLA), Digital Light Processing (DLP), Fused deposition modeling (FDM), Selective Laser Sintering (SLS), Selective laser melting (SLM), Electronic Beam Melting (EBM), Laminated object manufacturing (LOM), Binder jetting (BJ), Material Jetting (MJ) or Wax Casting (WC), or a combination thereof.

The scaffold, including the 3D printed scaffold, for example, may be manufactured by using a formulation comprising polycaprolactone dimethacrylate (PCLDA), calcium phosphate, HA, TCP, polyethylene glycol diacrylate (PEGDA), gelatin methacryloyl (GeIMA), or a mixture thereof.

This bone regeneration product/formulation can be used in regenerating bone. Such as bone regeneration method may comprise implanting the bone regeneration product/formulation in or across a bone defect. This bone defect may be any bone defect, such as a bone defect of a long bone. The size of this bone defect may be any size. For example, the size of this bone defect may be a critical size. The bone defect may be formed due to a congenital bone malformation. The congenital bone defect may be a defect related to a cleft lip and/or a cleft palate. The bone defect may be formed because of surgery, accident, and/or disease. This bone defect may be formed as a result of surgery carried out to treat craniosynostosis.

### Pharmaceutical Composition

The present application further provides a composition comprising (i) a carrier and (ii) one or more BMPR1A⁺ cells, or one or more spheres described above, or cells (such as progeny cells) derived from the one or more spheres. The composition can be a pharmaceutical composition where the carrier is a pharmaceutically acceptable carrier.

A composition for pharmaceutical use, e.g., a scaffold or implant with cells and/or factors, can include, depending on the formulation desired, pharmaceutically-acceptable, non-toxic carriers of diluents, which are defined as vehicles commonly used to formulate pharmaceutical compositions for animal or human administration. The diluent can be selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide (e.g., a growth factor), the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical composition described herein, e.g., SSCs alone or in combinations with various factors, can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

Data obtained from cell culture and/or animal studies can be used in formulating a range of dosages for humans. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxin, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing animal data, and other information available for the agent, a clinician can determine the maximum safe dose for an individual, depending on the route of administration. For instance, an intravenously administered dose may be more than a locally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Mammalian species that may be treated with the present methods include canines and felines; equines; bovines; ovines; etc. and primates, particularly humans. Animal models, particularly small mammals, e.g. murine, lagomorpha, etc. may be used for experimental investigations.

### Uses

The cells, compositions, formulations, and products disclosed herein can be used for various purposes including treating related disorders and in tissue engineering.

In some embodiments, the cells, compositions, formulations, and products disclosed herein can be used in the treatment of a subject, such as a human patient, in need of bone or cartilage replacement therapy. Examples of such subject can be subjects suffering from conditions associated with the loss of cartilage or bone from osteoporosis, osteoarthritis, genetic defects, disease, etc. Patients having diseases and disorders characterized by such conditions will benefit greatly from a treatment protocol of the pending claimed invention.

An effective amount of the pharmaceutical composition is the amount that will result in an increase in the number of chondrocytes, skeletal cells, cartilage or bone mass at the site of implant, and/or will result in a measurable reduction in the rate of disease progression in vivo. For example, an effective amount of a pharmaceutical composition will increase bone or cartilage mass by at least about 5%, at least about 10%, at least about 20%, preferably from about 20% to about 50%, and even more preferably, by greater than 50% (e.g., from about 50% to about 100%) as compared to the appropriate control, the control typically being a subject not treated with the composition.

The methods described above can be used in combined therapies with, e.g. therapies that are already known in the art to provide relief from symptoms associated with the aforementioned diseases, disorders and conditions. The combined use of a pharmaceutical composition described herein and these other agents may have the advantages that the required dosages for the individual drugs are lower, and the effect of the different drugs complementary.

In some embodiments, an effective dose of SSCs described herein, preferably SuSCs, are provided in an implant or scaffold for the regeneration of skeletal or cartilaginous tissue. An effective cell dose may depend on the purity of the population. In some embodiments, an effective dose delivers a dose of cells of at least about 10², about 10³, about 10⁴, about 10⁵, about 10⁶, about 10⁷, about 10⁸, about 10⁹ or more cells, which stem cells may be present in the cell population at a concentration of about 1%, about 5%, about 10%, about 20%, about 30%, about 40%, about 50%, about 60%, about 70%, about 80%, about 90% or more.

The present application provides methods and compositions for the differentiation of SSCs, including SuSCs, into cells such as chondrocytes. The cells produced by the methods are useful in providing a source of fully differentiated and functional cells for research, transplantation, and development of tissue engineering products for the treatment of human disease and traumatic injury repair.

Tissue Engineering

Tissue engineering is the use of a combination of cells, engineering and materials methods, and suitable biochemical and physico-chemical factors to improve or replace biological functions. For example, cells may be implanted or seeded into an artificial structure capable of supporting three-dimensional tissue formation. These structures, referred to herein as a matrix or scaffold, allow cell attachment and migration, deliver and retain cells and biochemical factors, enable diffusion of vital cell nutrients and expressed products. A high porosity and an adequate pore size are important to facilitate cell seeding and diffusion throughout the whole structure of both cells and nutrients. Biodegradability is often a factor since scaffolds may be absorbed by the surrounding tissues without the necessity of a surgical removal. The rate at which degradation occurs has to coincide as much as possible with the rate of tissue formation: this means that while cells are fabricating their own natural matrix structure around themselves, the scaffold is able to provide structural integrity within the body and eventually it will break down leaving the neotissue, newly formed tissue which will take over the mechanical load. Injectability is also important for clinical uses.

Many different materials (natural and synthetic, biodegradable and permanent) have been investigated and can be used for tissue engineering matrix or scaffold. Examples include Puramatrix, polylactic acid (PLA), polyglycolic acid (PGA) and polycaprolactone (PCL), and combinations thereof. Scaffolds may also be constructed from natural materials, e.g. proteins such as collagen, fibrin, etc; polysaccharidic materials, such aschitosan; alginate, glycosaminoglycans (GAGs) such as hyaluronic acid, etc. Functionalized groups of scaffolds may be useful in the delivery of small molecules (drugs) to specific tissues. Another form of scaffold under investigation is decellularised tissue extracts whereby the remaining cellular remnants/extracellular matrices act as the scaffold.

Treatment Methods

The above-described cells, compositions, formulations, products, and methods can be used to treat various bone defects such as large craniofacial bone defects, craniosynostosis, and bone fractures. Examples include (A) Bone fracture caused by various conditions, e.g. osteoporosis, and osteopenia, (B) Large bone defects caused by various conditions, including cancer surgeries, congenital malformation (e.g., Cleidocranial Dysplasia, Cleft Palate, Facial Cleft, Treacher-Collins, fibrous dysplasia), trauma, and progressive deforming diseases, and (C) the stem cell-based therapy may be used to substitute any procedure involving bone graft.

Large craniofacial bone defects, which are caused by various conditions, including trauma, infection, tumors, congenital disorders, and progressive deforming diseases, are major health issues (1). The autologous bone graft is a recommended procedure for extensive skeletal repairs but their success remains highly challenging owing to several limitations (1, 2). Consequently, alternative approaches have been explored (3, 4). Stem cell-based therapy is particularly attractive and promising, in light of the characterization of skeletal stem cells in craniofacial and body skeletons (5-11). Craniofacial bone is mainly formed through intramembranous ossification, a process different from the endochondral ossification required for the body skeleton (12). Because of the distinct properties of the stem cells of the craniofacial and body skeletons (5, 13), it is necessary to study each type of skeletal stem cells. Suture stem cells (SuSCs) are the stem cell population that is naturally programmed to form intramembranous bones during craniofacial skeletogenesis (5). The lack of a cell surface marker for stem cell isolation and the inability to maintain sternness characteristics ex vivo are two critical hurdles that restrict further advances in the field of skeletal regeneration. The cells, compositions, and methods disclosed herein address this unmet need.

Craniosynostosis, which affects one in ˜2,500 individuals, is one of the most common congenital deformities and is caused by premature suture closure (14). The suture serving as the growth center for calvarial morphogenesis is the equivalent of the growth plate in the long bone. Excessive intramembranous ossification caused by genetic mutation promotes suture fusion (15). An example is the genetic loss of function of AXIN2, which causes craniosynostosis in mice and humans (16, 17). In 2010, The inventors found that craniosynostosis can also be caused by mesenchymal cell fate switching, leading to suture closure through endochondral ossification (18). By regulating the interplay between bone morphogenetic protein (BMP) and fibroblast growth factor (FGF) pathways, Axin2-mediated Wnt signaling determines skeletogenic commitment into an osteogenic or chondrogenic lineage. The multipotency further supports the existence of skeletal stem cells within the suture mesenchyme (18). Because Axin2 expression in the presumptive niche site was tightly linked to suture patency, the inventors identified Axin2-expressing SuSCs as essential for calvarial development, homeostasis, and injury-induced repair (5). The Axin2-positive (Axin2+) SuSCs qualified for the modern, rigorous stem cell definition: They exhibit not only long-term self-renewal, clonal expansion, differentiation, and multipotency but also the ability to repair skeletal defects by direct engraftment and replacement of damaged tissue.

When used for tissue engineering or treatment methods, the SSCs and/or combinations of factors for lineage differentiation can be provided for in vivo use in a cellular solution, in which hydrating solutions, suspensions, or other fluids that contain the cells or factors that are capable of differentiating into bone or cartilage.

Bone or cartilage graft devices and compositions may be provided that are optimized in terms of one or more compositions, bioactivity, porosity, pore size, protein binding potential, degradability or strength for use in both load bearing and non-load bearing cartilage or bone grafting applications. Preferably, graft materials are formulated so that they promote one or more processes involved in bone or cartilage healing which can occur with the application of a single graft material: chondrogenesis, osteogenesis, osteoinduction, and osteoconduction. Chondrogenesis is the formation of new cartilaginous structures. Osteogenesis is the formation of new bone by the cells contained within the graft. Osteoinduction is a chemical process in which molecules contained within the graft (for example, bone morphogenetic proteins and TGF-β) convert the patient's or other bone progenitor cells into cells that are capable of forming bone. Osteoconduction is a physical effect by which the matrix of the graft forms a scaffold on which bone forming cells in the recipient are able to form new bone.

Inclusion of the factors and/or cells described herein can be used to facilitate the replacement and filling of cartilage or bone material in and around pre-existing structures. In some embodiments, the cells produce chondrocytes first, followed by the deposition of extra cellular matrix and bone formation. The bone grafts can provide an osteoconductive scaffold comprising calcium phosphate ceramics which provide a framework for the implanted progenitor cells and local osteocytes to differentiate into bone forming cells and deposit new bone. The use of calcium phosphate ceramics can provide for a slow degradation of the ceramic, which results in a local source of calcium and phosphate for bone formation. Therefore, new bone can be formed without calcium and phosphate loss from the host bone surrounding the defect site. Calcium phosphate ceramics are chemically compatible with that of the mineral component of bone tissues. Examples of such calcium phosphate ceramics include calcium phosphate compounds and salts, and combinations thereof.

In some embodiments, the cells and/or factors can be prepared as an injectable paste. A cellular suspension can be added to one or more cells to form an injectable hydrated paste. The paste can be injected into the implant site. In some embodiments, the paste can be prepared prior to implantation and/or store the paste in the syringe at sub-ambient temperatures until needed. In some embodiments, application of the composite by injection can resemble a bone cement that can be used to join and hold bone fragments in place or to improve adhesion of, for example, a hip prosthesis, for replacement of damaged cartilage in joints, and the like. Implantation in a non-open surgical setting can also be performed.

In other embodiments, the cells and/or factors can be prepared as formable putty. A cellular suspension can be added to one or more powdered minerals to form a putty-like hydrated graft composite. The hydrated graft putty can be prepared and molded to approximate any implant shape. The putty can then be pressed into place to fill a void in the cartilage, bone, tooth socket or another site. In some embodiments, graft putty can be used to repair defects in non-union bone or in other situations where the fracture, hole or void to be filled is large and requires a degree of mechanical integrity in the implant material to both fill the gap and retain its shape.

The methods described herein can be used for treating a cartilage or bone lesion, or injury, in a human or other animal subjects, comprising applying to the site a composition comprising cells and/or factors described herein, which may be provided in combinations with cements, factors, gels, etc. As referred to herein such lesions include any condition involving skeletal, including cartilaginous, tissue that is inadequate for physiological or cosmetic purposes. Such defects include those that are congenital, the result of disease or trauma, and consequent to surgical or other medical procedures. Such defects include, for example, a bone defect resulting from injury, defect brought about during the course of surgery, osteoarthritis, osteoporosis, infection, malignancy, developmental malformation, and bone breakages such as simple, compound, transverse, pathological, avulsion, greenstick and communuted fractures. In some embodiments, a bone defect is a void in the bone that requires filling with a bone progenitor composition.

The cells described herein can also be genetically altered in order to enhance their ability to be involved in tissue regeneration, or to deliver a therapeutic gene to a site of administration. To that end, a vector can be designed using the known encoding sequence for a desired gene, operatively linked to a promoter that is either pan-specific or specifically active in the differentiated cell type. Of particular interest are cells that are genetically altered to express a bone morphogenic protein, such as BMP-2 or BMP-4. See WO 99/39724. Production of these or other growth factors at the site of administration may enhance the beneficial effect of the administered cell, or increase proliferation or activity of host cells neighboring the treatment site.

Research Uses and Drug Screening

The cells described herein can also be used as a research or drug discovery tool, for example, to evaluate the phenotype of a genetic disease, e.g. to better understand the etiology of the disease, to identify target proteins for therapeutic treatment, to identify candidate agents with disease-modifying activity, e.g. to identify an agent that will be efficacious in treating the subject. For example, a candidate agent may be added to a cell culture comprising the SSC derived from a subject, and the effect of the candidate agent assessed by monitoring output parameters such as survival, the ability to form bone or cartilage, and the like, by methods described herein and in the art.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Examples of candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents can comprise cyclical carbon or heterocyclic structures and/or aromatic or polyaromatic structures substituted with one or more of the above functional groups. Candidate agents can be biomolecules, including peptides, polynucleotides, saccharides, fatty acids, steroids, purines, pyrimidines, derivatives, structural analogs or combinations thereof. Further examples include pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition.

In an example, the cells and methods described herein are useful for screening candidate agents for activity in modulating cell conversion into cells of a skeletal or chondrogenic lineage, e.g. chondrocytes, osteoblasts, or progenitor cells thereof. In screening assays for biologically active agents, the cells can be contacted with a candidate agent of interest in the presence of the cell reprogramming or differentiation system or an incomplete cell reprogramming or differentiation system, and the effect of the candidate agent is assessed by monitoring output parameters such as the level of expression of genes specific for the desired cell type, as is known in the art, or the ability of the cells that are induced to function like the desired cell type; etc. as is known in the art.

### Kits

Provided here are kits for identifying or isolating or enriching a suture stem cell or a skeletal stem cell.

The kit may comprise an agent that is specific to the BMPR1A protein or mRNA transcript. In one example, the kit comprises a system for contacting a biological sample comprising one or more an anti-BMPR1A antibody or an antigen-binding fragment thereof, or a ligand of BMPR1A or a BMPR1A-binding fragment thereof, and instructions for use thereof. In one embodiment, the kit further comprises a culturing system (e.g., a cell culture medium or a cell culture device or both) for culturing, maintaining, or expanding the population of cells, and instructions for use thereof. The kit may comprise one or more agents for measuring the BMPR1A protein level.

The agents may be, for example, reagents for carrying out analysis methods for detecting a protein or measuring protein levels. Examples of such analysis methods include, but are not limited to, FACS, immunohistostaining assay, Western blotting, ELISA, radioimmunoassay, radioimmunodiffusion, Ouchterlony immunodiffusion assay, rocket immunoelectrophoresis assay, immunoprecipitation assay, complement fixation assay, protein chip assay, etc. This kit may comprise reagents that detect antibodies forming antigen-antibody complexes, for example, labeled secondary antibodies, chromophores, enzymes (e.g., conjugated with antibodies) and their substrates. In addition, it may comprise an antibody specific to a control protein for quantification. The amount of antigen-antibody complexes formed may be quantitatively determined by measuring the signal intensity of a detection label. Such a detection label may be selected from the group consisting of, but not necessarily limited to, enzymes, fluorescent substances, ligands, luminescent substances, microparticles, redox molecules and radioactive isotopes.

For detecting mRNA transcripts, a kit may contain reagents for extracting mRNA, measuring the expression level of mRNA of BMPR1A (e.g., probes or PCR primers). The mRNA expression can be measured by any one selected from the group consisting of, but not limited to, in situ hybridization, polymerase chain reaction (PCR), reverse transcription-polymerase chain reaction (RT-PCR), real-time PCR, RNase protection assay (RPA), microarray, and Northern blotting.

Also provided is a kit comprising SSCs, spheres, a composition, a product, or a formulation described herein. Optionally, the kit contains one or more of the scaffold described herein. The SSC, spheres, composition, product, or formulation be presented in a kit, pack or dispenser, which may contain one or more unit dosage forms containing the active ingredient. The kit, for example, may comprise metal or plastic foil, such as a blister pack. The kit, pack, or dispenser may be accompanied by instructions for administration.

### Definitions

The terms “polypeptide” or “protein” are used interchangeably herein to refer to a polymer of amino acid residues and their derivatives. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally occurring and non-recombinant cell; or it is produced by a genetically engineered or recombinant cell, and comprise molecules having the amino acid sequence of the native protein, or molecules having deletions from, additions to, and/or substitutions of one or more amino acids of the native sequence. The terms “polypeptide” and “protein” specifically encompass antigen binding proteins, antibodies, or sequences that have deletions from, additions to, and/or substitutions of one or more amino acids of an antigen-binding protein. The term “polypeptide fragment” refers to a polypeptide that has an amino-terminal deletion, a carboxyl-terminal deletion, and/or an internal deletion as compared with the full-length protein. Such fragments may also contain modified amino acids as compared with the full-length protein. In certain embodiments, fragments are about five to 500 amino acids long. For example, fragments may be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains.

The term “isolated polypeptide” refers to a polypeptide that has been separated from at least about 50 percent of polypeptides, peptides, lipids, carbohydrates, polynucleotides, or other materials with which the polypeptide is naturally found when isolated from a source cell. Preferably, the isolated polypeptide is substantially free from any other contaminating polypeptides or other contaminants that are found in its natural environment that would interfere with its therapeutic, diagnostic, prophylactic or research use.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is composed of a heavy chain variable region (abbreviated herein as V_{H}) and a heavy chain constant region. The heavy chain constant region is composed of three domains, C_{H}1, C_{H}2 and C_{H}3. Each light chain is composed of a light chain variable region (abbreviated herein as V_{L}) and a light chain constant region. The light chain constant region is composed of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

Accordingly, the terms “antibody” and “antibodies” include full-length antibodies, antigen-binding fragments of full-length antibodies, and molecules comprising antibody CDRs, V_{H} regions or V_{L} regions. Examples of antibodies include monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), scFv-Fcs, camelid antibodies (e.g., llama antibodies), camelized antibodies, affybodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies disclosed herein refer to polyclonal antibody populations. Antibodies can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies disclosed herein are IgG antibodies, or a class (e.g., human IgG₁ or IgG₄) or subclass thereof. In a specific embodiment, the antibody is a humanized monoclonal antibody.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L} and CHI domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993)); (iv) a Fd fragment consisting of the V_{H} and CHI domains; (v) a Fv fragment consisting of the V_{L} and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds to a specific antigen is substantially free of antibodies that specifically bind antigens other than the specific antigen). An isolated antibody can be substantially free of other cellular material and/or chemicals.

As used herein, the term “administering” refers to the delivery of compositions of the present invention by any suitable route. Cells can be administered a number of ways including, but not limited to, parenteral (such term referring to intravenous and intra-arterial as well as other appropriate parenteral routes), intrathecal, intraventricular, intraparenchymal, intracisternal, intracranial, intrastriatal, intranigral, intranasal, intraperitoneal, intramuscular, subcutaneous, intradermal, transdermal, or transmucosal administration, among others which term allows cells to migrate to the ultimate target site where needed. Multiple units of cells can be administered simultaneously or consecutively (e.g., over the course of several minutes, hours, or days) to a patient.

The terms “grafting” and “transplanting” and “graft” and “transplantation” are used to describe the process by which cells are delivered to the site where the cells are intended to exhibit a favorable effect, such as repairing damage to a patient's bone or cartilage. Cells can also be delivered in a remote area of the body by any mode of administration as described above, relying on cellular migration to the appropriate area to effect transplantation.

The term “therapeutic composition” or pharmaceutical composition” refers to the combination of an active agent with a carrier, inert or active, making the composition especially suitable for diagnostic or therapeutic use in vivo or ex vivo.

As used herein, “therapeutic cells” refers to a cell population that ameliorates a condition, disease, and/or injury in a patient. Therapeutic cells may be autologous (i.e., derived from the patient), allogeneic (i.e., derived from an individual of the same species that is different from the patient) or xenogeneic (i.e., derived from a different species than the patient). Therapeutic cells may be homogenous (i.e., consisting of a single cell type) or heterogeneous (i.e., consisting of multiple cell types). The term “therapeutic cell” includes both therapeutically active cells as well as progenitor cells capable of differentiating into a therapeutically active cell.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc.) and a human). The term “subject” includes human and non-human animals. The preferred subject for treatment is a human. As used herein, the terms “subject” and “patient” are used interchangeably irrespective of whether the subject has or is currently undergoing any form of treatment. In one embodiment, the subject is a human. In another embodiment, the subject is an experimental, non-human animal or animal suitable as a disease model.

The term “patient” is used herein to describe an animal, preferably a human, to whom treatment, including prophylactic treatment, with the cells according to the present invention, is provided. The term “donor” is used to describe an individual (animal, including a human) who or which donates cells or tissue for use in a patient.

The term “primary culture” denotes a mixed cell population of cells from an organ or tissue within an organ. The word “primary” takes its usual meaning in the art of tissue culture.

A “tissue” refers to a group or layer of similarly specialized cells which together perform certain special functions.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. The terms “prevent,” “preventing,” “prevention,” “prophylactic treatment” and the like refer to reducing the probability of developing a disorder or condition in a subject, who does not have, but is at risk of or susceptible to developing a disorder or condition. “Ameliorating” generally refers to the reduction in the number or severity of signs or symptoms of a disease or disorder.

A “prophylactic treatment” includes a treatment administered to a subject who does not display signs or symptoms of a condition such that treatment is administered for the purpose of diminishing, preventing, or decreasing the risk of developing the condition. A “therapeutic treatment” includes a treatment administered to a subject who displays symptoms or signs of a condition and is administered to the subject for the purpose of reducing the severity or progression of the condition. A therapeutic treatment can also partially or completely resolve the condition.

An “effective amount” generally means an amount which provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

As used herein, the term “stem cells” refer to cells with the ability to both replace themselves and to differentiate into more specialized cells. Their self-renewal capacity generally endures for the life-span of the organism. A pluripotent stem cell can give rise to all the various cell types of the body. A multipotent stem cell can give rise to a limited subset of cell types. For example, a hematopoietic stem cell can give rise to the various types of cells found in blood, but not to other types of cells. Multipotent stem cells can also be referred to as somatic stem cells, tissue stem cells, lineage-specific stem cells, and adult stem cells. The non-stem cell progeny of multipotent stem cells are progenitor cells (also referred to as restricted-progenitor cells). Progenitor cells give rise to fully differentiated cells, but a more restricted set of cell types than stem cells. Progenitor cells also have comparatively limited self-renewal capacity; as they divide and differentiate they are eventually exhausted and replaced by new progenitor cells derived from their upstream multipotent stem cell.

The term “skeletal stem cell” refers to a multipotent and self-renewing cell capable of generating bone marrow stromal cells, skeletal cells, and chondrogenic cells. By self-renewing, it is meant that when they undergo mitosis, they produce at least one daughter cell that is a skeletal stem cell. By multipotent it is meant that it is capable of giving rise to progenitor cell (skeletal progenitors) that give rise to all cell types of the skeletal system. They are not pluripotent, that is, they are not capable of giving rise to cells of other organs in vivo.

Skeletal stem cells can be reprogrammed from non-skeletal cells, including without limitation mesenchymal stem cells, and adipose tissue containing such cells. Reprogrammed cells may be referred to as induced skeletal stem cells, or iSSC. “iSSC” arise from a non-skeletal cell by experimental manipulation. Induced skeletal cells have characteristics of functional SSCs derived from nature, that is, they can give rise to the same lineages. Human SSC cell populations can be negative for expression of certain markers such as CD45, CD235, Tie2, and CD31; and positively express others, such as podoplanin (PDPN). The mouse skeletal lineage can characterized as CD45−, Ter119−, Tie2−, αv integrin+. The mouse SSC can be further characterized as Thy1−6C3− CD105− CD200+.

Suture stem cells (SuSCs) refer to a population of skeletal stem cells from the suture mesenchyme that exhibit long-term self-renewal, clonal expansion, and multipotency. These SuSCs reside in the suture midline and serve as the skeletal stem cell population responsible for calvarial development, homeostasis, injury repair, and regeneration. Suture stem cells are the stem cell population that is naturally programmed to form intramembranous bones during craniofacial skeletogenesis.

Chondrocytes (cartilage cells)” refers to cells that are capable of expressing characteristic biochemical markers of chondrocytes, including but not limited to collagen type II, chondroitin sulfate, keratin sulfate and characteristic morphologic markers of smooth muscle, including but not limited to the rounded morphology observed in culture, and able to secrete collagen type II, including but not limited to the generation of tissue or matrices with hemodynamic properties of cartilage in vitro.

As used herein, the phrase “maintaining stem cells” refers not just to culturing the stem cells in a manner preserving their viability, but also to retaining their functionality as stem cells, that is, to being self-renewing and capable of giving rise to the full range of progenitor lineages appropriate to the particular type of stem cell (these two functions together “regenerative activity”). One way of demonstrating that stem cells have been successfully maintained is through an engraftment experiment in which all the appropriate cell types (bearing a genetic marker distinguishing them from the host) are observed to arise from the graft and remain present over an extended period of time, for example 4 months.

As used herein, the phrase “expanding stem cells” refers not just to maintaining the stem cells but to culturing the stem cells in a manner that the number of stem cells in the culture increases. One way of demonstrating that stem cells have been successfully expanded is an engraftment experiment comparing the percentage of donor-derived cells obtained from transplants of cultured and freshly isolated stem cells. The comparison is based on transplanting the same number of freshly isolated stem cells as were originally placed in culture. An increased percentage of donor-derived cells in the recipients of the cultured stem cells as compared to in the recipients of the freshly-isolated stem cells is consistent with the successful expansion of the stem cells in culture.

A “marker” or “biomarker” is a molecule useful as an indicator of a biologic state in a subject. The marker or biomarkers disclosed herein can be polypeptides that exhibit a change in expression or state, which can be correlated with the development, differentiation, or fate of a cell. In addition, the markers disclosed herein are inclusive of messenger RNAs (mRNAs) encoding the marker polypeptides, as measurement of a change in expression of an mRNA can be correlated with changes in expression of the polypeptide encoded by the mRNA. As such, determining an amount of a biomarker in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

In the context of skeletal stem cells, a “marker” or “biomarker” means that, in cultures or tissues comprising cells that have been programmed to become skeletal stem cells, the marker is expressed only by the cells of the culture or tissue that will develop, are developing, and/or have developed into skeletal stem cells. It will be understood by those of skill in the art that the stated expression levels reflect detectable amounts of the marker protein or mRNA on or in the cell. A cell that is negative for staining (the level of binding of a marker-specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive.”

Standard gene/protein nomenclature guidelines generally stipulate gene name abbreviations/symbols for human, as well as non-human primates, domestic species, and default for everything that is not a mouse, rat, fish, worm, or fly, are capitalized and italicized (e.g., BMPR1A) and protein name abbreviations are capitalized, but not italicized (e.g., BMPR1A). Further, standard gene/protein nomenclature guidelines generally stipulate mouse, rat, and chicken gene name abbreviations/symbols italicized with the first letter only capitalized (e.g., Bmpr1a) and protein name abbreviations capitalized, but not italicized (e.g., Bmpr1a).

In contrast, the gene/protein nomenclature used herein when referencing a specific biomarker uses one with all capital letters (e.g., BMPR1A) or one having lower letters (e.g., Bmpr1a) for the biomarker abbreviation, is intended to be inclusive of genes (including mRNAs and cDNAs) and proteins across animal species. That is, when referring to a maker in this application, the name abbreviations/symbols in capital letters and in non-capital letters are used interchangeably unless their context indicates otherwise (e.g., in the working examples below, related figures, and related descriptions). Accordingly, the exemplary human or mouse biomarkers described herein are not intended to limit the present subject matter to human or mouse polypeptide biomarkers or mRNA biomarkers only. Rather, the present subject matter encompasses biomarkers across animal species that are associated with SSCs.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Unless otherwise stated, the term “about” means within an acceptable error range for the particular value.

### EXAMPLES

### Example 1 Study Design, Materials, and Methods

This example describes the study design, materials, and methods used in Examples 2-11 below.

Study Design

This study was designed to identify regulators of SuSCs. Using a genomic approach, the inventors found BMP signaling was potentially involved in SuSC regulation. The inventors generated loss-of-function mouse models to individually examine three BMP type I receptors, revealing that Bmpr1a was essential for SuSC-mediated calvarial morphogenesis. The inventors used an inducible knockout system to specifically disrupt Bmpr1a in Axin2+ SuSCs in developing mice and evaluated the effect of disruption on suture fusion and studied the mechanism of the aberrant suture closure. To test if stem cell depletion caused precocious differentiation that mediated aberrant suture closure, the inventors examined stemness, self-renewal, quiescence, stem cell frequency, proliferation, clonal expansion, and bone-generating ability of SuSCs using ex vivo culture and in vivo transplantation assays. To provide translation to humans, the inventors analyzed a human SuSC population and determined if BMPR1A served as a cell surface marker for isolation of both mouse and human SuSCs with characteristics of skeletal stem cells. To ensure scientific reproducibility, all studies were performed multiple times with proper controls, including wild-type mice or mice carrying appropriate transgene(s). Mice of both sexes were examined because of the potential sensitivity of skeletogenesis to sex hormones. For mouse studies, at least three or five mice were used for each group. For culture studies and transplantation studies, at least 3-5 independent experiments were performed. Human samples were obtained from non-syndromic synostosis patients with at least five independent samples used for this study. The analysis of samples by μCT was performed by a technician who was blinded to the condition. No randomization, statistical method to predetermine the sample size or inclusion/exclusion-defining criteria for samples were used.

Animals and Models

The Axin2-rtTA, TRE-H2BGFP, TRE-Cre, R26RTomato, Bmpr1a^{Fx}, Acvr1^{Fx}, Bmpr1b^{-/-}, SCID mouse strains, and genotyping methods were reported previously (18, 25, 46-54). To create the Axin2^{GFP} strain (18, 55), mice carrying Axin2-rtTA and TRE-H2BGFP transgenes were obtained and treated with Dox (2 mg/ml plus 50 mg/ml sucrose) for 3 days as described (46, 47, 52). The Axin2^{Cre-Dox} mouse strain was generated by obtaining mice carrying Axin2-rtTA and TRE-Cre transgenes. The Axin2^{Cre-Dox} mice were then crossed with Bmpr1a^{Fx}, Acvr1^{Fx}, and R26RTomato mice to obtain Bmpr1a^{Ax2}, Acvr1^{Ax2}, and Axin2^{Cre-Dox}; R26RTomato mouse, respectively. The expression of Cre in the Axin2-expressing cells was then induced by Dox treatment (18, 56). Both male and female mice were used in this study. Care and use of experimental animals described in this work comply with guidelines and policies of the University Committee on Animal Resources at the University of Rochester.

Cell Isolation and Purification

Primary suture mesenchymal cells containing SuSCs were isolated from mouse calvaria as described (5). Briefly, an approximately 1.5 mm width tissue containing the sagittal suture and the adjacent parietal bones was dissected, and the suture was separated from the parietal bone parts. Suture parts were incubated with 0.2% collagenase in PBS (pH 7.0 to 7.6, #21-031-CV, CORNING) at 37° C. for 1.5 hours. The dissociated cells were filtered using CELL STRAINER 40 μm Nylon (#352340, FALCON), and then resuspended in DMEM media for transplantation analysis, in DMEM containing 5% fetal bovine serum (FBS) for cell sorting, or in DMEM containing 25 μg/ml insulin, 100 μg/ml transferrin, 20 nM progesterone, 30 nM sodium selenite, 60 nM putrescine, 20 ng/ml EGF, 20 ng/ml bFGF, ng/ml B27 supplement, and 1% penicillin-streptomycin for sphere culture.

For in vitro culture as spheres, cells were grown for 7-10 days in ultra-low attachment surface plates (#2023-03-23, CORNING) during which time the cells formed spheres (1⁰). The spheres were dissociated with 0.25% trypsin-EDTA and seeded as a single-cell suspension in ultra-low attachment surface plates for the culture of the next passage (2⁰). This dissociation and replating were repeated for up to 5 passages (5⁰). For differentiation, the spheres were transferred to 24-well plates with treated surfaces (662160, GREINER BIO-ONE, Monroe, NC) to enhance attachment and cultured in differentiation α-MEM medium containing ascorbic acid (50 μg/ml) and 4 mM β-glycerophosphate for 3 weeks.

For human suture cell isolation, the inventors obtained calvarial discards containing unfused suture from patients with nonsyndromic craniosynostosis (3 to 14 months). Bone fragments were removed to obtain the suture mesenchyme and the tissue was incubated with 0.2% collagenase in PBS (pH 7.0 to 7.6, #21-031-CV, CORNING) for 1.5 h at 37° C. The dissociated cells were then filtered through a 40-pin strainer, followed by resuspension in DMEM media containing 20% FBS for sphere culture, or in PBS containing 3% FBS for cell purification.

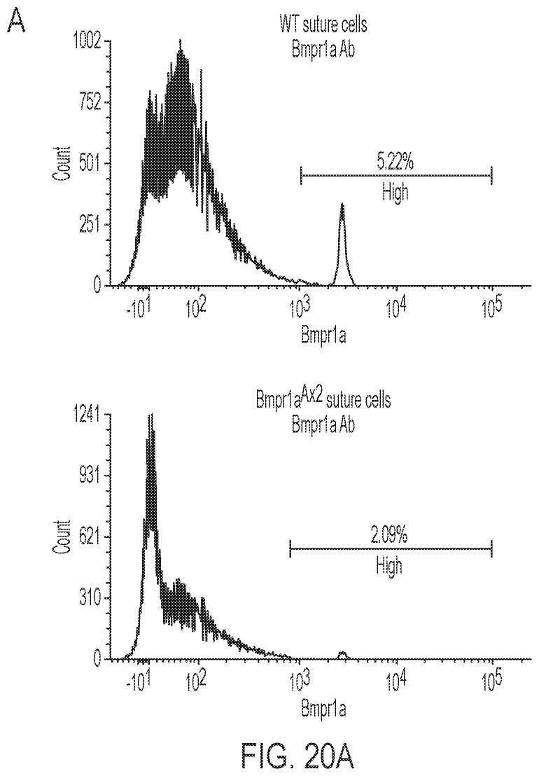

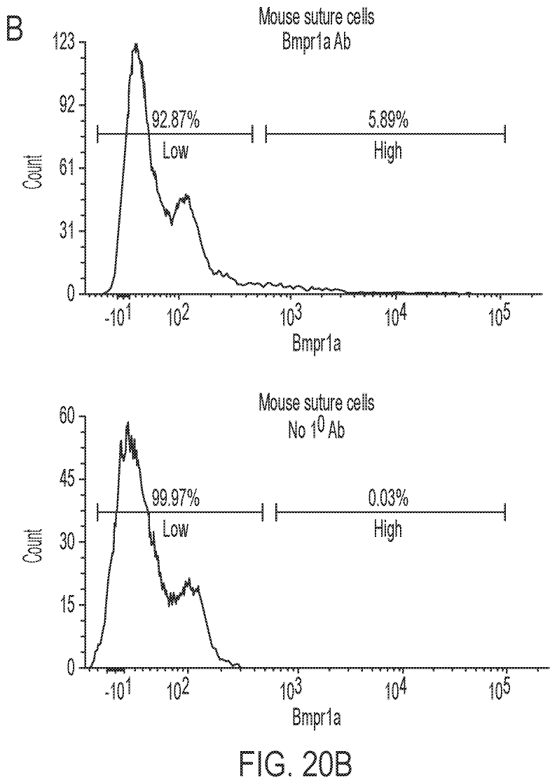

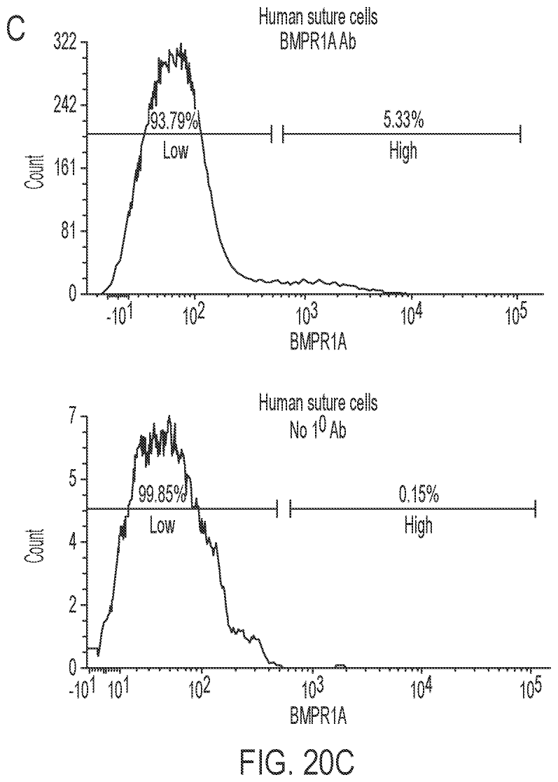

To purify Bmpr1a+ and Bmpr1a− cell populations, freshly isolated suture cells were stained with primary mouse monoclonal Bmpr1a antibody (MA5-17036, THERMO FISHER SCIENTIFIC, Waltham, MA), followed by sorting according to the intensity of secondary antibody-conjugated Texas Red using FACSAria-II (BD Biosciences, San Jose, CA). The specificity of this Bmpr1a antibody for the isolation of cells with high amounts of Bmpr1a was determined by fluorescence activated cell sorting (FACS) (FIG. 20).

Kidney Capsule Transplantation

The transplantation of freshly isolated suture cells or cultured sphere cells into the kidney capsule was performed as described (5). Freshly isolated cells were obtained from P5 mice. For limiting dilution analysis, 10²-10⁵ cells were transplanted. The frequency of stem cells was calculated with Extreme Limiting Dilution Analysis (ELDA) software with validation of the likelihood ratio test for a single-hit model (57). Spheres were tested from 1⁰ and 3⁰ passages by transplanting 30 spheres per kidney capsule.

Staining and Analysis

Skull preparation, fixation, and embedding for paraffin and frozen sections were performed as described (16, 18, 56, 58). Samples were subject to hematoxylin/eosin staining for histology, Goldner's Trichrome staining, GFP analysis, β-gal staining, van Kossa staining, or immunological staining with avidin:biotinylated enzyme complex (16, 18, 46, 47, 58-62). For antigen retrieval, samples were incubated with antigen unmasking solution (H3300, VECTOR) in pressured cooking for 10 min or 20 mM Tris-HCl (pH 9) for 16 hr at 70° C. For in vitro deletion of Bmpr1a, cells isolated from mouse Bmpr1a^{Fx/Fx} suture were infected by Lenti-GFP or Lenti-Cre viruses (MOI=1). The whole-mount von Kossa staining, immunological staining, in situ hybridization, and double labeling analyses were performed as described (5, 58, 63). For double labeling of von Kossa staining and immunostaining, samples were fixed with 2% paraformaldehyde and 0.02% NP-40 for 1 hour at room temperature, followed by incubation with 1% silver nitrate under ultraviolet light for 30 min, and with 5% sodium thiosulfate for 5 minutes. Then, the stained samples were processed for paraffin sections and subsequent immunological staining.

To detect proliferating cells, EdU was added to the sphere for 16 hours after four-day culture, followed by attachment using CYTOSPIN (THERMO FISHER SCIENTIFIC). After fixing with 95% ethanol for 5 min on ice and 2% paraformaldehyde for 20 min at room temperature, the spheres were treated with 0.5% TRITON-X100 for 10 min and incubated with EdU reaction buffer for 30 min according to the manufacturer's protocol (THERMO FISHER SCIENTIFIC). Rabbit polyclonal antibodies OSTERIX (ab22552, ABCAM, Cambridge, MA; 1:200), Bmpr1a (ABP-PAB-10536, ALLELE, San Diego, CA; 1:100), phospho-Tak1 (arb191688, BIORBYT, St Louis, MO; 1:200), phospho-ERK1/2 (4370, CELL SIGNALING TECHNOLOGY, 1:50); rabbit monoclonal antibodies Ki67 (RM-9106, THERMO FISHER SCIENTIFIC; 1:200), Axin2 (2151, CELL SIGNALING TECHNOLOGY; 1:500), phospho-p38 MAPK (4511, CELL SIGNALING TECHNOLOGY, 1:200), phospho-JNK (4668, CELL SIGNALING TECHNOLOGY, 1:100) were used for immunostaining. The Bmpr1a antibody (MA5-17036, THERMO FISHER SCIENTIFIC, 1:200) was used for FASC sorting and immunostaining studies. Images were taken using a ZEISS AXIO OBSERVER microscope (CARL ZEISS, Thornwood, NY) or LEICA DM2500 microscope with a DFC7000T digital imaging system (LEICA BIOSYSTEMS Inc., Buffalo Grove, IL).

Statistical Analysis

R software version 3.2.1 or MICROSOFT EXCEL 2010 was used for statistical analysis. The significance was determined by two-sided student's t-tests. A p-value of less than 0.05 was considered statistically significant. Before performing the t-tests, the normality of the data distribution was first validated by the Shapiro-Wilk normality test. The activity of signaling pathways in SuSCs was estimated by the active Z score using IPA software (Ingenuity® Systems). Statistical data were presented as mean±SEM or SD. The stem cell frequency was examined by Extreme Limiting Dilution Analysis (ELDA) software with validation of the likelihood ratio test for a single-hit model (57).

### Example 2 Identification of a BMP Pathway in Axin2-Expressing SuSCs

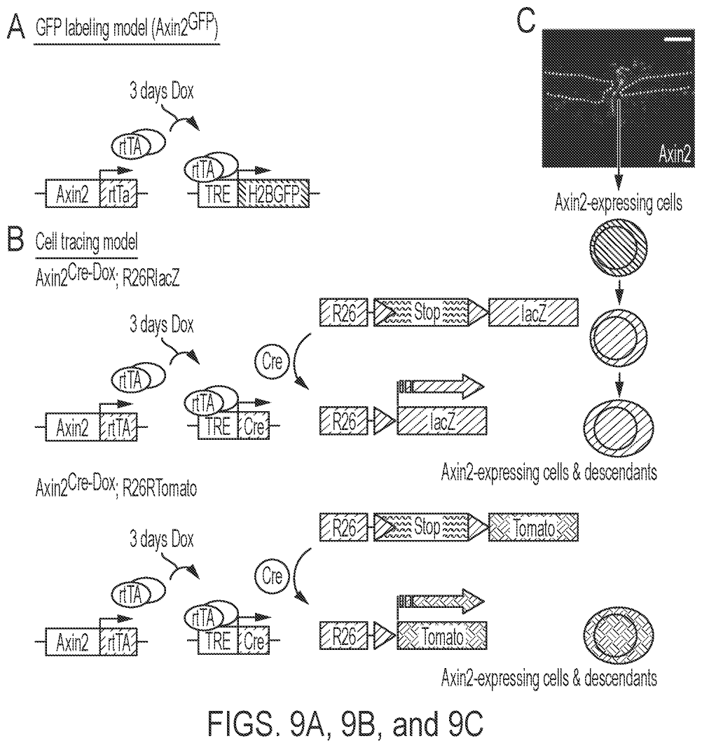

To identify and isolate Axin2-positive (Axin2+) cells in the suture mesenchyme and to track the descendants of these cells, the inventors genetically engineered mice that inducibly express a green fluorescent protein (GFP) to reflect the activity from the Axin2 promoter in a spatiotemporal-specific manner (Axin2^{GFP}) (FIG. 9A). Using a similar system to inducibly expression of Cre to drive either lacZ for β-galactosidase (β-gal) labeling or the fluorescent protein Tomato, the inventors established mouse models permitting the tracing of Axin2+ cells (FIG. 9B). From Axin2^{GFP} mice at postnatal day 28 (P28), the inventors isolated the Axin2+ cell population with high-intensity GFP signal (Axin2⁺/GFP^{hi}) (FIG. 9C) and the non-expressing cell population that is negative for GFP (Axin2⁻/GFP⁻) from the suture mesenchyme.

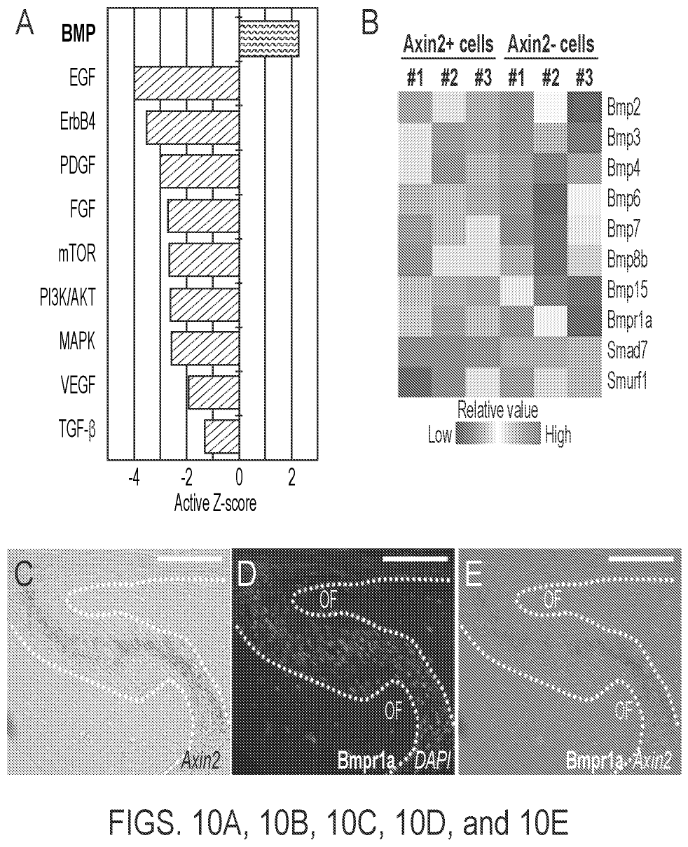

Microarray analysis comparing SuSCs (Axin2+) and non-SuSCs (Axin2−) revealed approximately 9,000 genes with significant differences (p-value <0.05, n=3). With pathway analysis using INGENUITY PATHWAY (IPA) software, The inventors obtained two scores: an enrichment score, representing the statistically significant accumulation of genes in each pathway, and a Z-score, representing the activation state of the signal by matching observed and predicted patterns of upregulation and downregulation (19). In SuSCs, most of the identified signaling pathways were inactive but the BMP pathway exhibited significant activation (FIG. 10A, p-value <10⁻¹³, z-score >2.3). Detailed analysis of expression of genes encoding BMP signaling components showed that seven BMP ligands and the type I receptor Bmpr1a are upregulated and two negative regulators (Smad7 and Smurf1) are downregulated in SuSCs (FIG. 10B). The results suggested that BMP ligands signal through Bmpr1a to activate the pathway in SuSCs. Therefore, The inventors examined SuSCs for Bmpr1a in the Axin2^{Cre}; R26RlacZ model in which SuSCs are marked by lacZ (FIG. 9B). Double labeling identified Bmpr1a in Axin2+ SuSCs at P28 (FIG. 10C-E), consistent with a role for BMP Bmpr1a signaling in SuSC regulation.

### Example 3 Identification of a Requirement of Bmpr1a for SuSC-Mediated Calvarial Development and Homeostasis

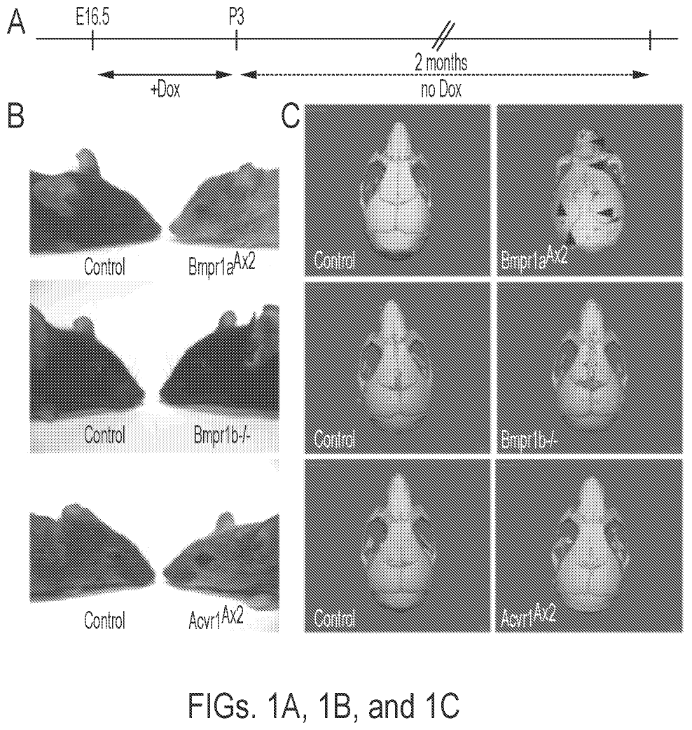

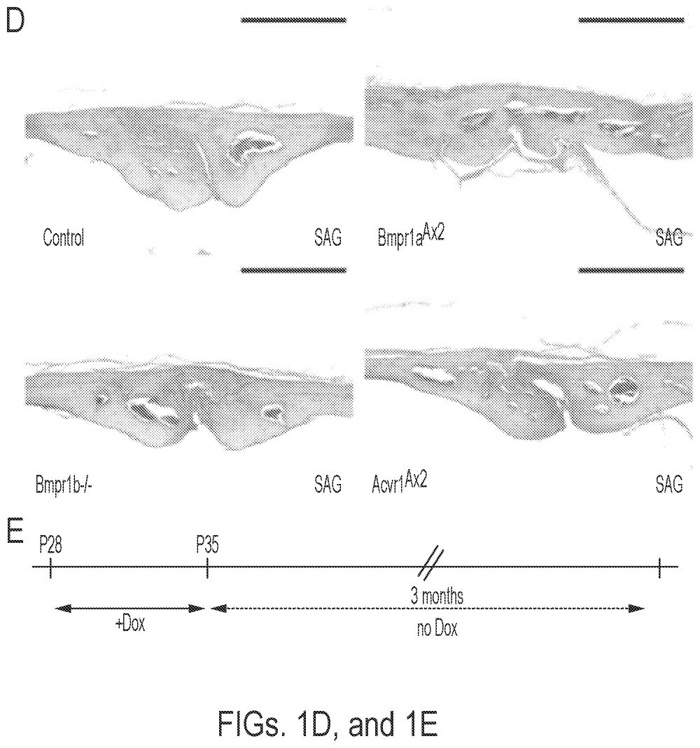

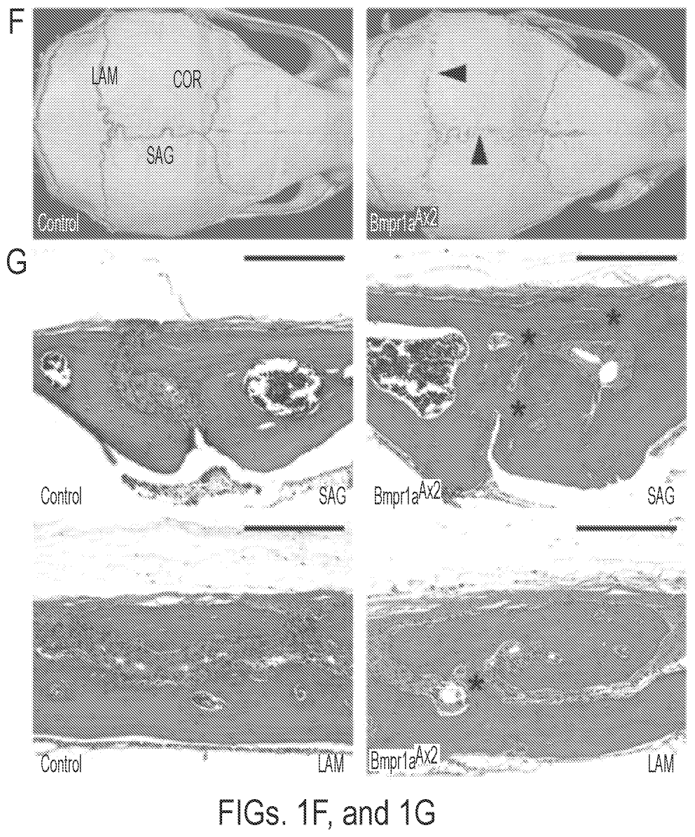

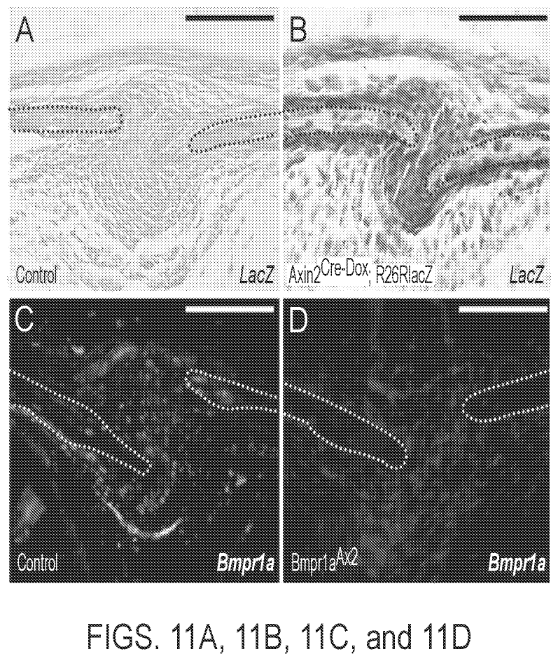

To delineate the functional importance of BMP signaling in SuSCs, The inventors studied the type I receptors in calvarial morphogenesis (20, 21). Most BMP family members signal through one of three type I receptors, Bmpr1a, Bmpr1b, and Acvr1 (20, 21). The inventors focused on the receptors because there are many BMP ligands, making genetic studies challenging. Mice with global inactivation of Bmpr1b are viable, whereas the null mutation of Bmpr1a or Acvr1 is associated with embryonic lethality due to defective mesodermal formation (22-25). Therefore, the inventors developed Bmpr1a^{Ax2} (Axin2^{Cre-Dox}; Bmpr1a^{Fx/Fx}) and Acvr1^{Ax2} (Axin2^{Cre-Dox}; Acvr1^{Fx/Fx}) models, enabling doxycycline (Dox)-inducible deletion of Bmpr1a or Acvr1 in the Axin2+ SuSCs. For studying the calvarial formation, Dox was administered from embryonic day (E) 16.5 to P3 to initiate Cre-dependent gene deletion (FIG. 1A). The efficiency of Cre-mediated recombination in Axin2+ SuSCs and their descendent cells was demonstrated using an R26RlacZ reporter strain (FIG. 11A, B). Immunostaining showed not only the efficacy of Bmpr1a ablation in the mutant but also the specificity of the Bmpr1a antibody (FIG. 11C, D).

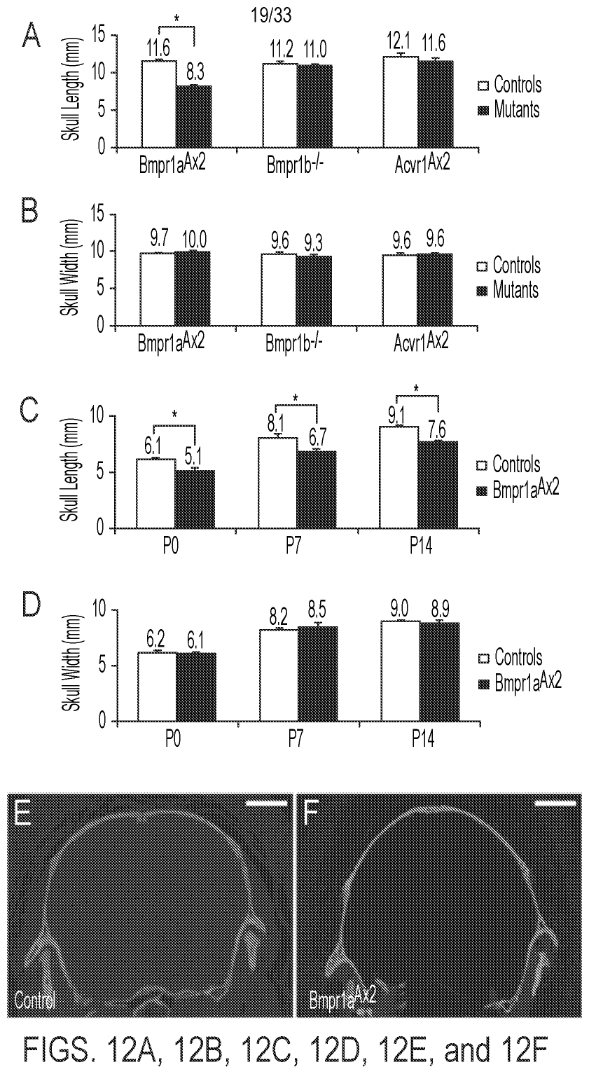

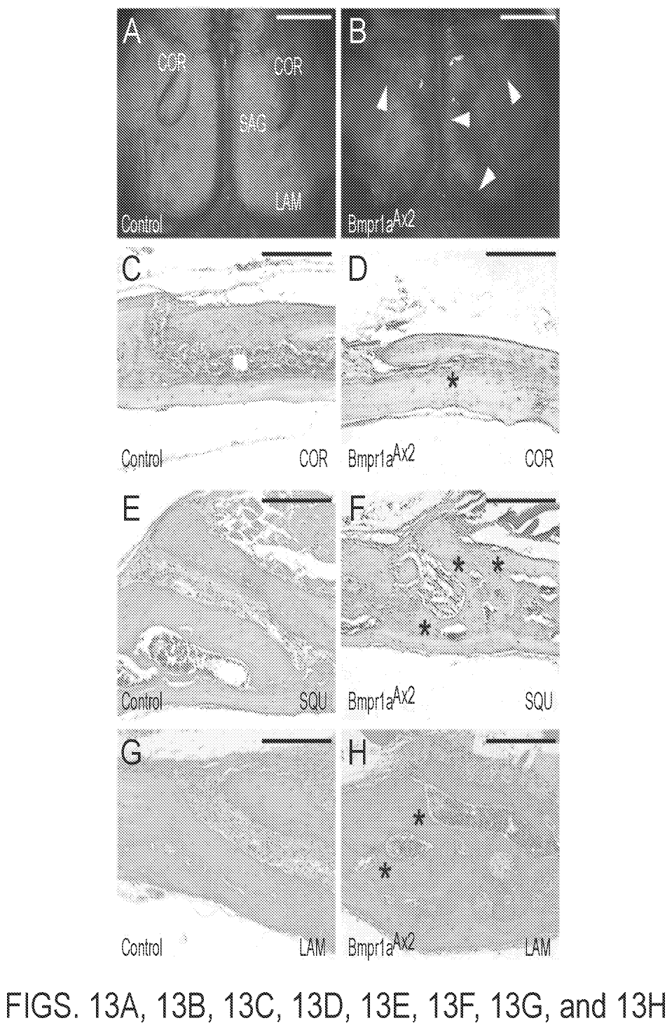

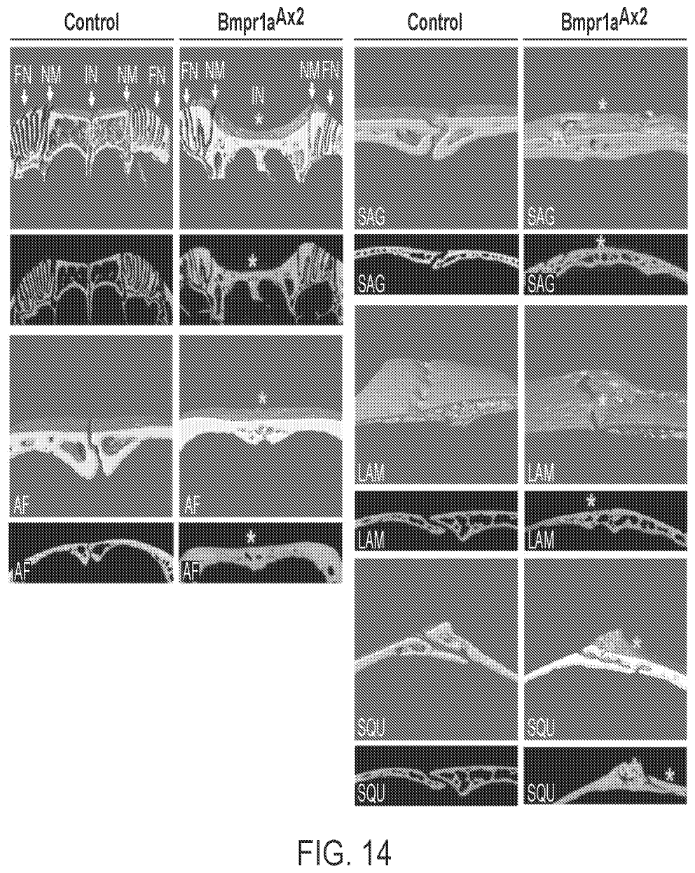

Bmpr1a^{Ax2}, but not Bmpr1b^{-/-} or Acvr1^{Ax2}, mice displayed craniofacial anomalies at 2 months (FIG. 1B and FIGS. 12A-B). Indeed, the Bmpr1a mutants were easy to identify by the abnormal skull shape. Micro-computed tomography (μCT) analysis and histology revealed calvarial bone and suture closure abnormalities that were specifically caused by the loss of Bmpr1a (FIG. 1C, D and FIG. 12). The Bmpr1a^{Ax2} skull was significantly shorter without any significant difference in width throughout the first 14 days of postnatal development (FIGS. 12A-D, p-value <0.05). Consequently, the skulls of the Bmpr1a^{Ax2} mutant mice were dome-shaped compared to the flatter shape of the skulls of the control mice (FIGS. 12E, F). Analysis of skulls stained with alizarin red (FIGS. 13A, B), histological analysis (FIGS. 13C-H), and μCT analyses (FIG. 14) revealed multiple synostoses in the internasal, anterior frontal, sagittal, lambdoid, and squamosal sutures of Bmpr1a^{Ax2} mice. The results indicated a specific requirement of Bmpr1a in SuSCs during calvarial morphogenesis.

The inventors previously demonstrated that Axin2+ cells function as skeletal stem cells in calvarial development and homeostasis (5). To test if Bmpr1a regulates adult SuSCs, the inventors induced its deletion in the mature skull. In humans, the growth of the skull reaches 90% of adult size in the first year, and 95% of adult size by 6 years of age (26). The skull size in teenagers is identical to that of adults. In mice, 90% of the skull development is completed at P28 where SuSCs are restricted to the suture midline (5, 27). Therefore, the inventors administrated Dox to the P28 Bmpr1a^{Ax2} mice for 7 days (FIG. 1E). Three months after the Dox treatment, the mutants were examined by μCT and histology. Deletion of Bmpr1a in adult SuSCs resulted in aberrant suture morphogenesis and multiple sutural synostoses (FIG. 1F, G), suggesting an essential role of Bmpr1a in SuSC-mediated calvarial homeostasis. Thus, together the analysis of mice lacking Bmpr1a during embryonic and early postnatal development along with those lacking Bmpr1a after skull maturation indicated that Bmpr1a was critical for both calvarial development and homeostasis.

### Example 4 Craniosynostosis is Initiated in the Midline of the Bmpr1a^{Ax2} Suture

A time-course study was performed to decipher the suture closure process. Dox-inducible deletion of Bmpr1a was conducted from E16.5-P3. Skulls from mice were evaluated by alizarin red staining (FIG. 2A) and Goldner's trichrome staining (FIG. 2B) at P0, P7, and P14 (FIG. 2). At P0, Bmpr1a deletion caused a wider suture. However, abnormal ossification within the suture mesenchyme was evident at P7 and P14 in the absence of Bmpr1a, ultimately leading to suture closure at 2 months (FIG. 1C, D). This finding suggested that aberrant ossification is initiated in the suture midline and moves toward the osteogenic fronts.

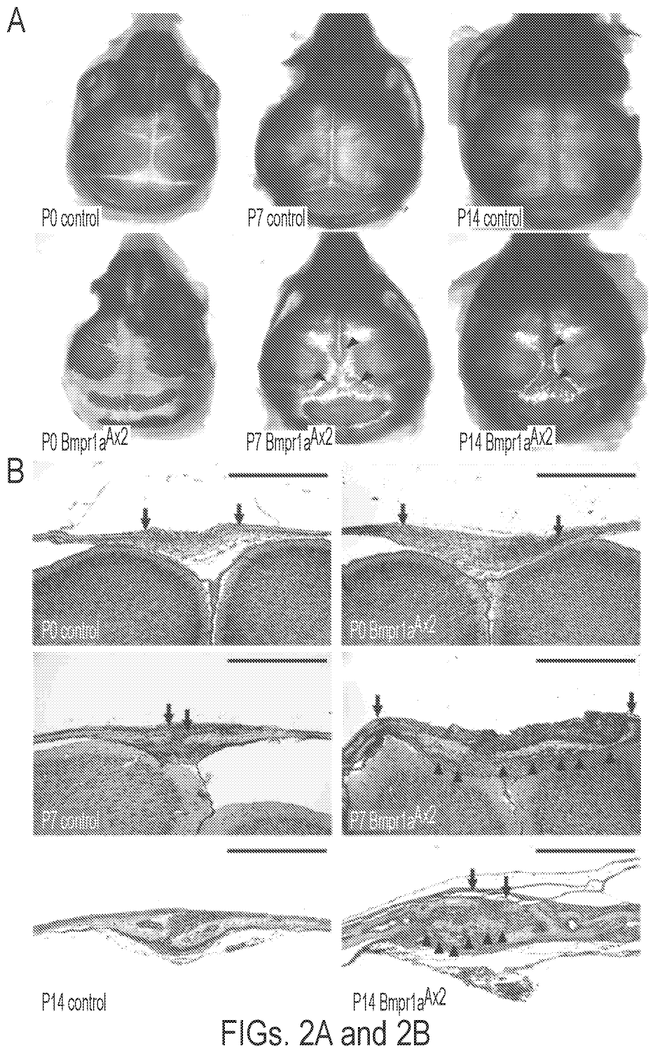

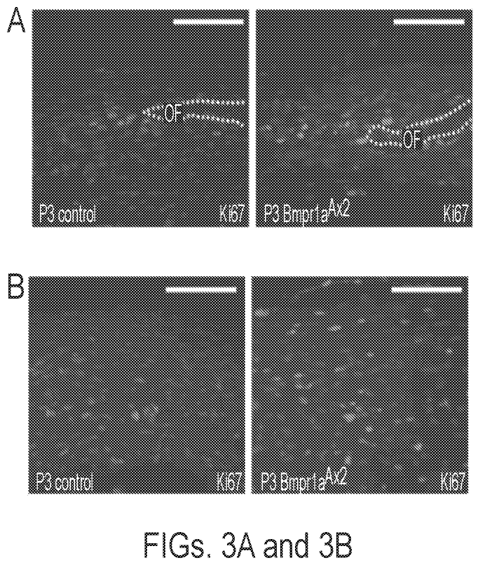

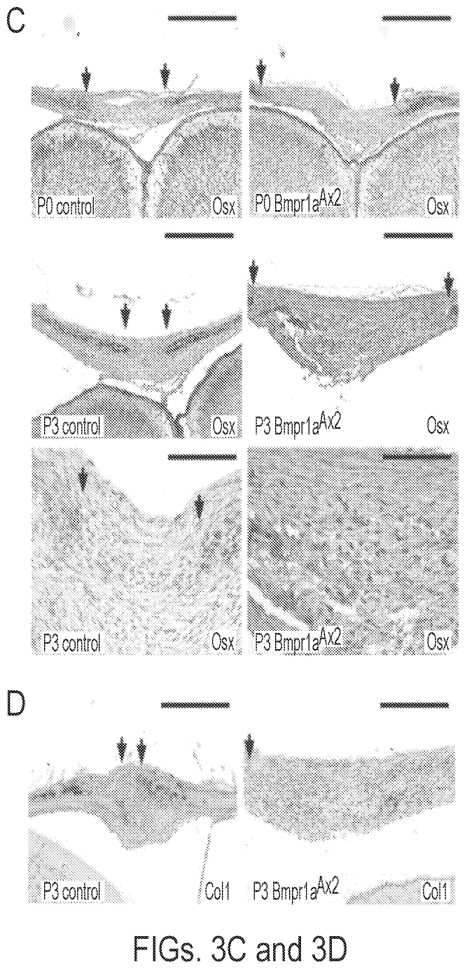

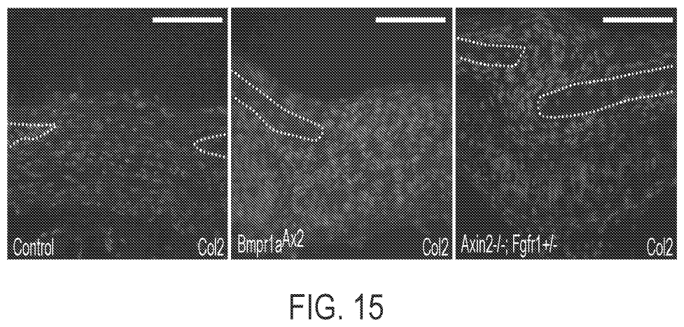

Calvarial bones are formed through osteoblast-mediated intramembranous ossification. To delineate the aberrant ossification process caused by the SuSC-specific deletion of Bmpr1a, the inventors examined osteoblast proliferation and differentiation. In the suture of control mice at P3, immunostaining of Ki67, a marker for cells undergoing mitosis, revealed that most cells are quiescent in the suture mesenchyme but actively proliferating at the osteogenic fronts (FIG. 3A), which is the site where intramembranous ossification occurs towards the suture midline. In the Bmpr1a^{Ax2} mice, the number of Ki67+ cells was aberrantly increased in the suture mesenchyme (FIG. 3B). To examine osteoprogenitor cells, the inventors immunostained for Osterix (Osx); to detect osteoblast cells, the inventors performed in situ hybridization of type I collagen (Col1). At P0, Osx+ osteoprogenitors were detected only at the osteogenic fronts of both control and Bmpr1a^{Ax2} mice (FIG. 3C). However, at P3, the inventors detected increased numbers of Osx+ osteoprogenitors in the suture mesenchyme in response to Bmpr1a deletion in SuSCs (FIG. 3C). Rather than clusters of Col1+ osteoblasts at the osteogenic fronts as were observed in control mouse calvaria; Col1+ osteoblasts were found throughout the suture mesenchyme of Bmpr1a^{Ax2} calvaria (FIG. 3D). Compared with sutures of Axin2^{-/-}; Fgfr^{1+/-} mice, no type II collagen (Col2)-positive chondrocytes were detected in the mutant (FIG. 15), suggesting that the loss of Bmpr1a function does not promote stem cell fate change and the aberrant suture closure was not caused by ectopic chondrogenesis and endochondral ossification. The findings indicated that aberrant ossification is initiated in the suture mesenchyme rather than the osteogenic fronts.

### Example 5 Signaling Effects of Bmpr1a on SuSCs in the Developing Suture

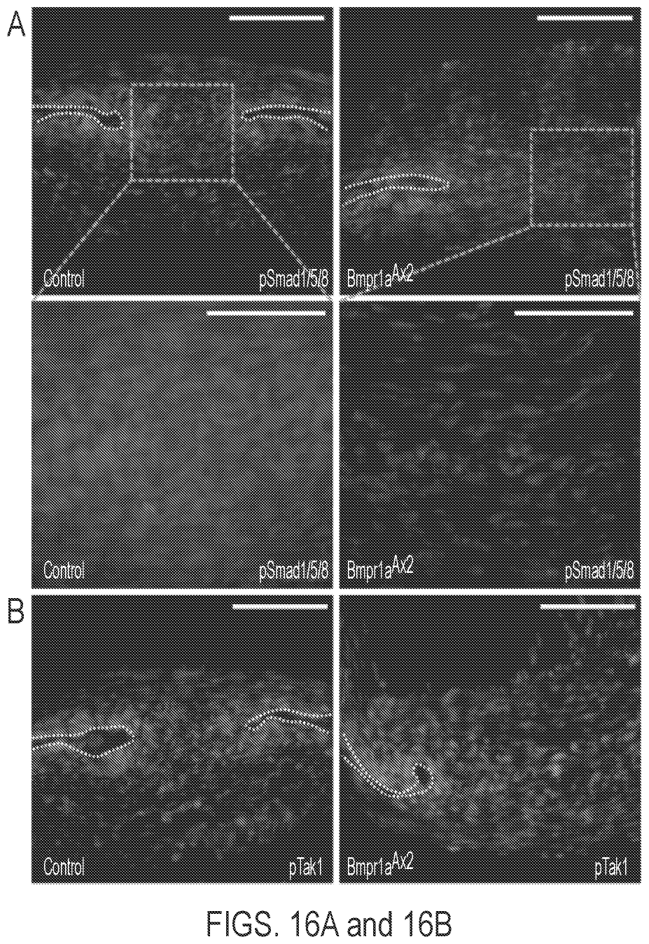

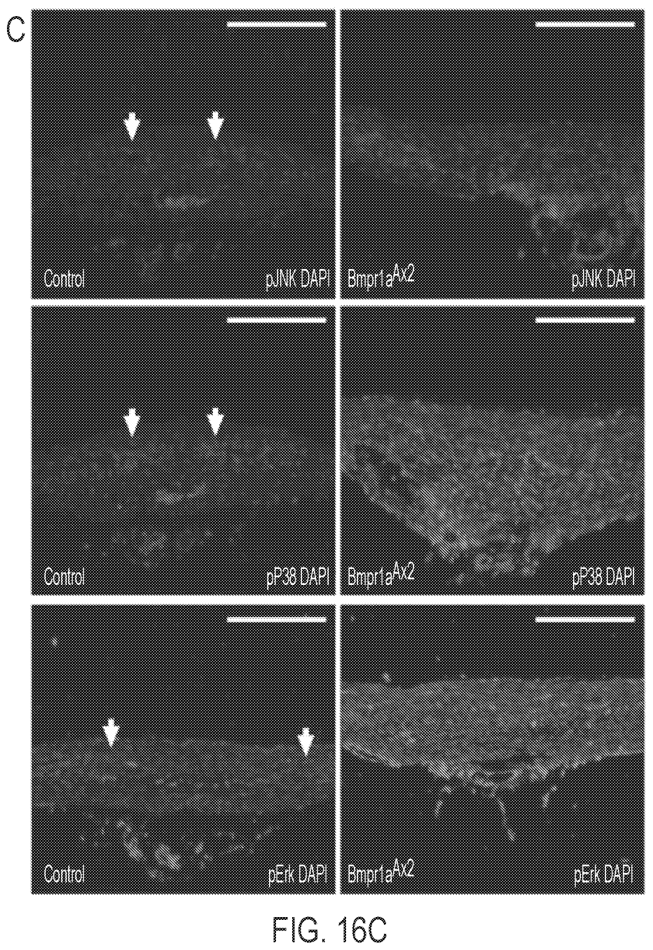

To examine the downstream pathways affected by the loss of Bmpr1a function in SuSCs, The inventors analyzed “canonical” BMP signaling mediated by Smad proteins (28), as well as “noncanonical” signaling through kinases (29). BMPs that signal through Bmpr1a activate the transcriptional regulators Smad1, Smad5, or Smad8, or some combination thereof (collectively, Smad1/5/8). Immunostaining showed comparable amounts of phosphorylated Smad1/5/8 in the osteogenic front and periosteum of control and Bmpr1a^{Ax2} (FIG. 16, upper). However, the amount of phosphorylated Smad1/5/8 appeared less in the Bmpr1a^{Ax2} suture midline (FIG. 16A, lower). Immunostaining of phosphorylated TAK1 indicated activation of this kinase mainly in the osteogenic front and similar amounts of phosphorylated TAK1 were present in the osteogenic front and suture region of control and Bmpr1a^{Ax2} mice (FIG. 16B). Examination of activation of mitogen-activated protein kinases (MAPKs) downstream of Tak1 showed strong activation (phosphorylation) of p38 but not of JNK (FIG. 16C). The other MAP kinase Erk was also abundant in the mutant (FIG. 16C). These results indicated that deletion of Bmpr1a reduced canonical but enhanced noncanonical signaling in SuSCs, leading to precocious differentiation in the suture midline and craniosynostosis.

### Example 6 Bmpr1a is Required for Maintenance of SuSCs

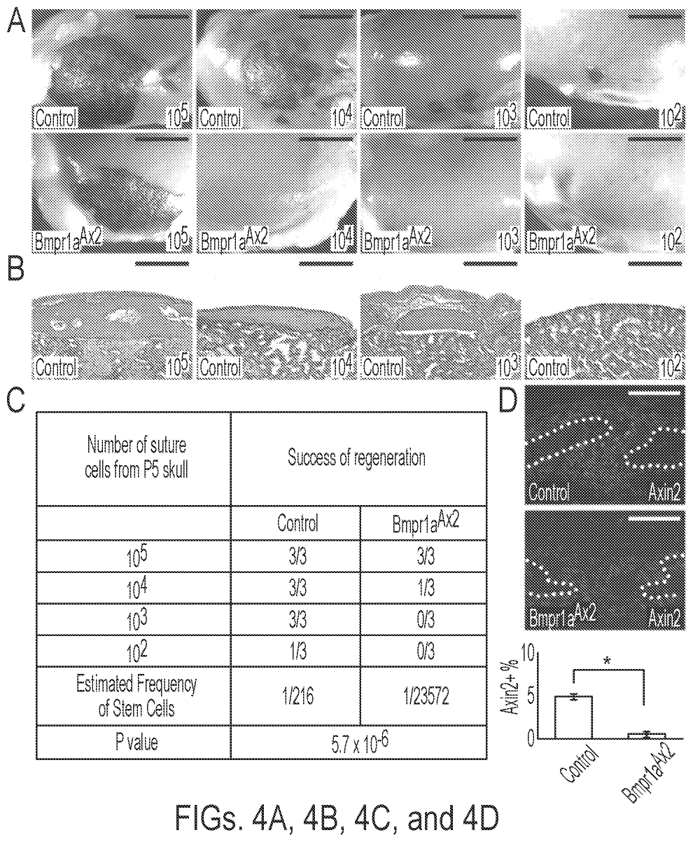

The enhanced cell proliferation and numbers of osteoprogenitors and osteoblasts in the Bmpr1a^{Ax2} suture mesenchyme (FIG. 3) implied a potential role of Bmpr1a in stem cell maintenance. The inventors hypothesized that the loss of Bmpr1a depletes the stem cell population leading to precocious osteoblast differentiation and intramembranous ossification. To test this hypothesis, the inventors examined SuSC characteristics of Bmpr1a^{Ax2}. By in vivo clonal expansion analysis, the inventors showed the ability of a single Axin2+ SuSC to generate calvarial bone upon implantation into the kidney capsule (5). With limiting dilution analysis, the inventors further established a quantitative method to examine stem cell clonal expansion in the transplanted kidney to measure stem cell frequency (5). The inventors used this assay to investigate if Bmpr1a deficiency affects the clonal expansion and number of SuSCs. Various amounts of cells isolated from the control and Bmpr1a^{Ax2} sutures from P5 mice were implanted into the kidney capsule, then the implanted site was evaluated by von Kossa staining to detect mineralized ectopic bones (FIG. 4A) and by histological analysis to examine the bone structure (FIG. 4B). Transplantation of 10³-10⁵ control cells had a 100% success rate on bone formation (FIG. 4C). At 10² cells from control mice, ectopic bone tissue formed although at a lower frequency than with the higher number of cells (FIG. 4C). In contrast, ectopic bone tissue was not detected in kidneys transplanted with 10²-10³ Bmpr1a^{Ax2} cells, and only at 10⁵ cells did all of the kidneys exhibit bone formation (FIG. 4C). Estimating stem cell frequency using ELDA software (33), the inventors found the loss of Bmpr1a significantly decreases SuSC frequency in the P5 suture (FIG. 4C; control: 1 in 216 cells and Bmpr1a^{Ax2}:1 in 23,572 cells, p-value=5.7×10⁻⁶).

Furthermore, immunostaining analysis revealed a significant loss of Axin2+ SuSCs in the Bmpr1a^{Ax2} suture (FIG. 4D; control, 4.9±0.3%; Bmpr1a^{Ax2} 0.6±0.2%, p-value <0.01). These data suggested that Bmpr1a plays an essential role in the maintenance of SuSC stemness such that the loss of Bmpr1a induces their precocious differentiation and aberrant ossification in the suture midline, leading to craniosynostosis.

### Example 7 Preservation of SuSC Stemness in Culture

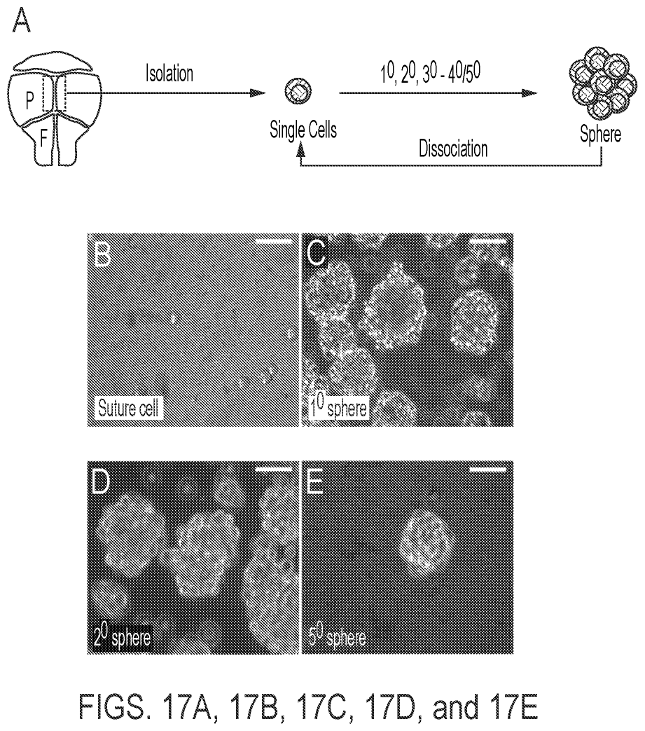

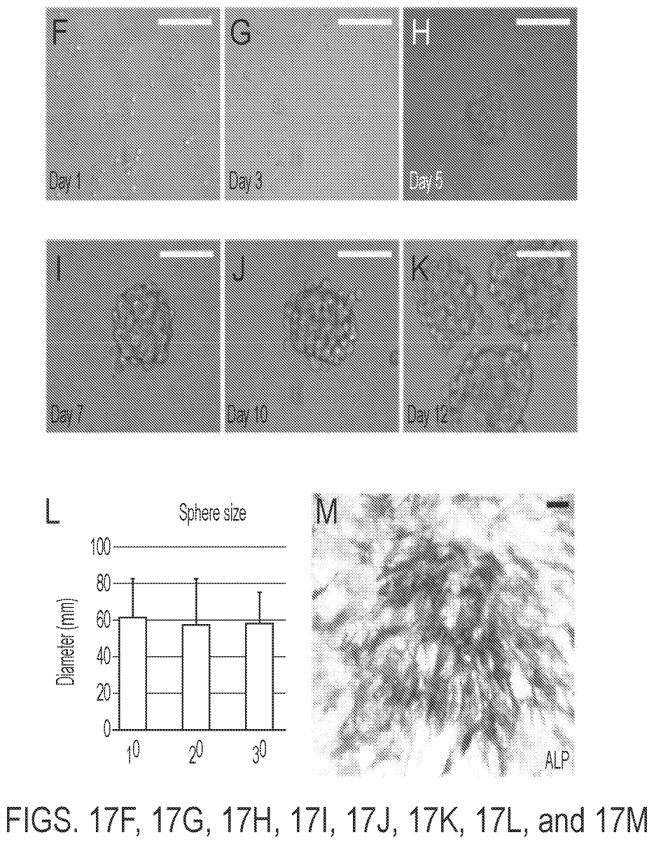

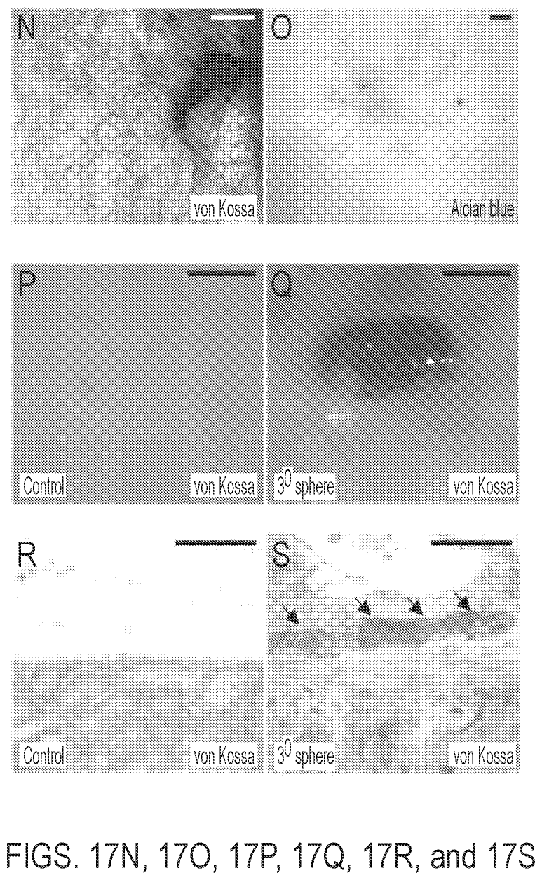

A protocol for maintaining SuSC sternness in vitro is needed because conventional culture methods for mesenchymal stromal cells (MSCs) do not preserve SuSC sternness. Sphere culture can maintain the properties of neural and mammary stem cells, recapitulating in vivo characteristics (34). The inventors established a culture protocol for cells isolated from the suture mesenchyme (FIG. 17A) that maintains stem cell characteristics. The inventors found that the isolated suture cells formed primary (1⁰) spheres when grown in single-cell suspension culture at very low seeding density (FIGS. 17B, C). When 1⁰ spheres were dissociated into single cells, the cells formed secondary (2⁰) spheres (FIG. 17D). For each passage, 10⁴ cells were seeded and suture cell spheres continued to form without notable decreases in number for up to 5 passages, implying the presence of SuSCs with self-renewing ability (FIG. 17E). The time-course analysis suggested that each sphere formed from a single cell (FIGS. 17F-K). The average sphere size remained comparable in different passages (FIG. 17L).

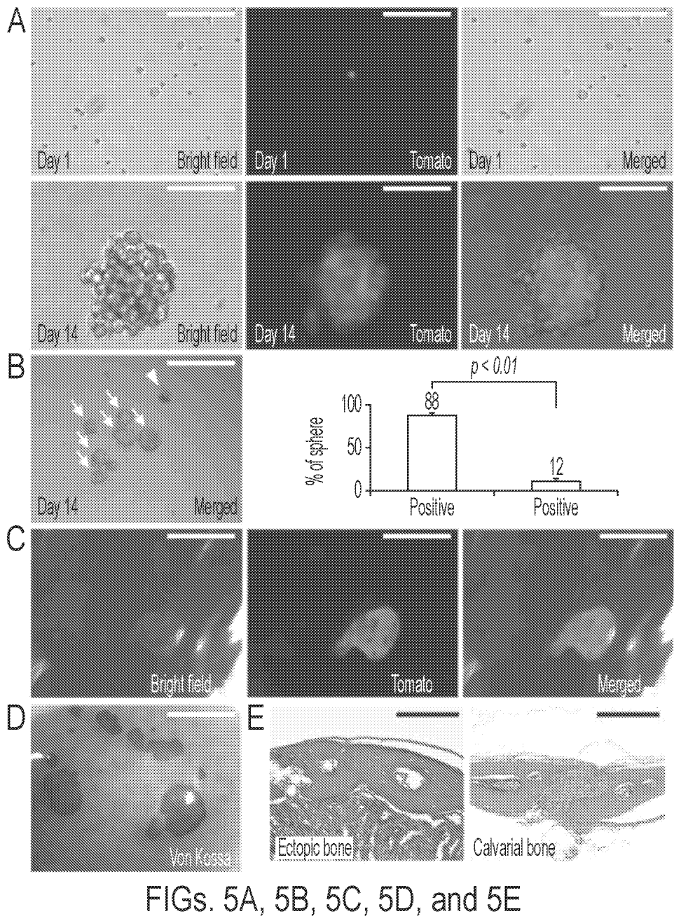

To determine the cellular origin of the sphere-forming cells, the inventors used the Axin2^{Cre-Dox}; R26RTomato model (FIG. 9B). Suture cells, isolated from the Axin2^{Cre-Dox}; R26RTomato mice with Dox treatment for 3 days from P7 to P10, were cultured in the absence of Dox. A small portion of cells was positive for Tomato at the beginning of the single-cell suspension culture (FIG. 5A, top row; 5±0.3%, n=3, mean±SEM). After 2 weeks, most spheres consisted of all Tomato+ cells, suggesting they derived from a single Axin2+ cell with clonal expansion ability (FIG. 5A, B). The inventors did not detect chimeric spheres with a mixture of Tomato+ and Tomato− cells, indicating that suture spheres are not formed by cell aggregation.

The inventors evaluated the multipotency of the suture spheres by culturing the spheres under conditions that promote differentiation. These multipotency tests showed that 3⁰ spheres differentiated into osteoblast cells and formed mineralized nodules or into chondrogenic cells (FIGS. 17M-O). To examine the clonal expansion and bone-forming abilities of suture spheres in vivo, the inventors performed kidney capsule transplantation analysis. The inventors implanted thirty spheres, which were formed from cells isolated from the Axin2^{Cre-Dox}; R26RTomato suture, into the kidney capsule. The spheres successfully expanded, colonized, and engrafted (FIG. 5C). Like transplanted freshly isolated suture cells undergoing intramembranous ossification (5), the transplanted 1⁰ spheres (FIG. 5D, E) or 3⁰ spheres (FIGS. 17P-S) generated bone tissue resembling calvarial bones (FIG. 5E). The results indicated that the newly developed culture system preserves SuSC sternness and differentiation properties, enabling their analyses in an ex vivo setting.

### Example 8 Ex Vivo Characterization of SuSCs

Previous in vivo examination of mouse SuSCs at 1-month-old, as well as data for P3 suture mesenchyme (FIG. 3A), indicated their quiescence (5). These quiescent SuSCs should be included with the isolation procedure disclosed herein for sphere culture. To test if a subpopulation of the isolated SuSCs exhibit quiescence in culture, the inventors performed pulse-chase labeling analyses by labeling the cells in vivo and then chasing them in culture. Using the Axin2^{Dox-GFP} (Axin2-rtTA; TRE-H2BGFP) mouse model (FIG. 9A), the inventors performed a pulse-chase analysis to examine Axin2+ SuSCs. Axin2-expressing cells were labeled in vivo with GFP by the administration of Dox from P7-P10 (5). Suture cells were isolated at P10 and cultured in the absence of Dox for sphere formation. GFP analysis of 1⁰ spheres revealed only a single cell with strong fluorescence intensity (FIG. 6A). Similar results were obtained in the subsequent 2⁰ and 3⁰ cultures (FIG. 6B-C). Although 32% of the 1⁰ spheres lacked any GFP+ cells, all spheres found in the 2⁰ and 3⁰ passages contained one strongly GFP+ cell (FIG. 6D). The presence of some GFP-negative spheres in the 1⁰ culture is likely due to the presence of skeletal precursors with limited proliferation ability that only generate spheres in the 1⁰ culture and not in the subsequent passages. The inventors hypothesized that the persistent presence of a single cell that is strongly GFP positive arises from asymmetric division; whereas symmetric division dilutes the GFP signal (FIG. 6E). The inventors confirmed that the GFP+ cell was also positive for Axin2 by immunofluorescence analysis (FIG. 6F). Labeling the spheres for proliferating cells showed that the GFP+ cell was not actively proliferating (FIG. 6G). These results support the hypothesis that SuSCs undergo asymmetric division in which one daughter cell remains undifferentiated, thus showing label-retaining ability, and the rest of the cells in the sphere arise from the other daughter cell (FIG. 6E).

### Example 9 Requirement of Bmpr1a for Self-Renewal and Bone Formation of SuSCs

Using ex vivo pulse-chase labeling analysis, the inventors examined the distribution of Bmpr1a in the spheres. Spheres immunostained for Bmpr1a showed that this receptor is present in the GFP+ cell and a few neighboring cells (FIG. 6H). These results are consistent with the in vivo double-labeling analysis showing only partial overlap of the Bmpr1a and Axin2 reporter signals (FIG. 10C-E). Next, the inventors examined the necessity of Bmpr1a for SuSC self-renewal using serial culture analysis. The culture of cells isolated from the P5 control and Bmpr1a^{Ax2} sutures showed comparable sphere formation in 1⁰ cultures (FIG. 6I). However, the number of 2⁰ and 3⁰ spheres was significantly reduced in cultures from the mutant mice, suggesting that the self-renewing ability of SuSCs is compromised by the loss of Bmpr1a (FIG. 6I, p-value <0.05, n=3, mean±SEM, student t-test). The size of the mutant spheres was also smaller compared to the control (FIG. 6J). Thus, the data indicated that Bmpr1a plays an essential role in SuSC self-renewal and maintenance of sternness properties in sphere culture.

Our prior study showed that SuSC self-renewal is linked to clonal expansion and bone regeneration in vivo, especially when a small number of cells are used for transplantation analysis (5). To test if clonal expansion and osteogenic abilities are affected in Bmpr1a-deficient SuSCs, the inventors implanted 30 spheres into the kidney capsule. In this assay, the inventors used 1⁰ spheres due to the impaired formation of the mutant spheres in subsequent passages (FIG. 6I). Ectopic bone formation mediated by Bmpr1a^{Ax2} 1⁰ suture spheres was severely impaired (FIG. 6K-N). In 1 of 3 transplants with mutant spheres, the inventors detected a tiny area that stained with von Kossa.

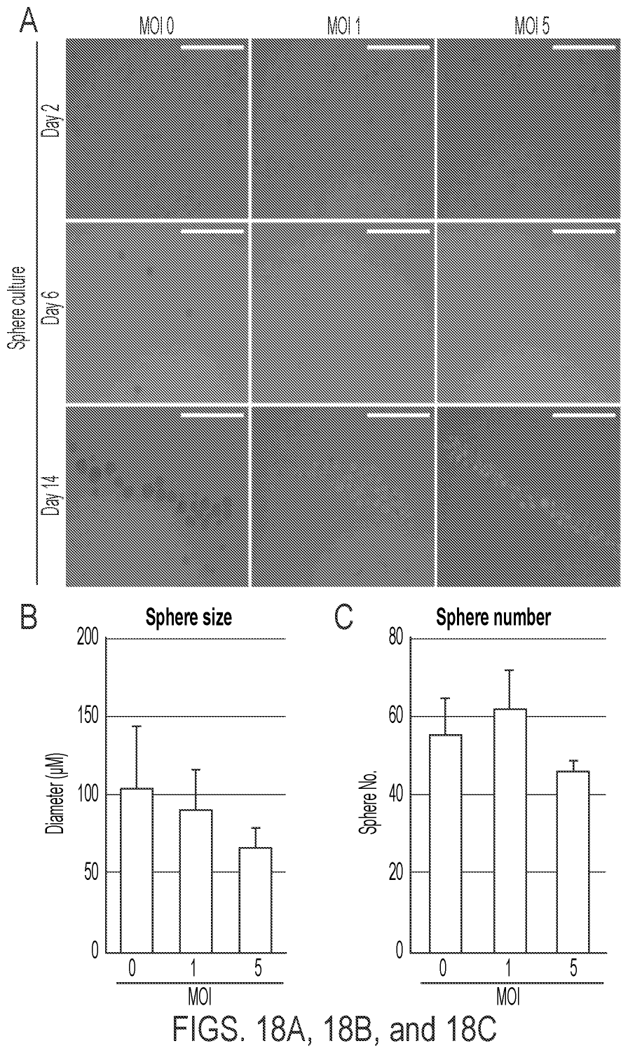

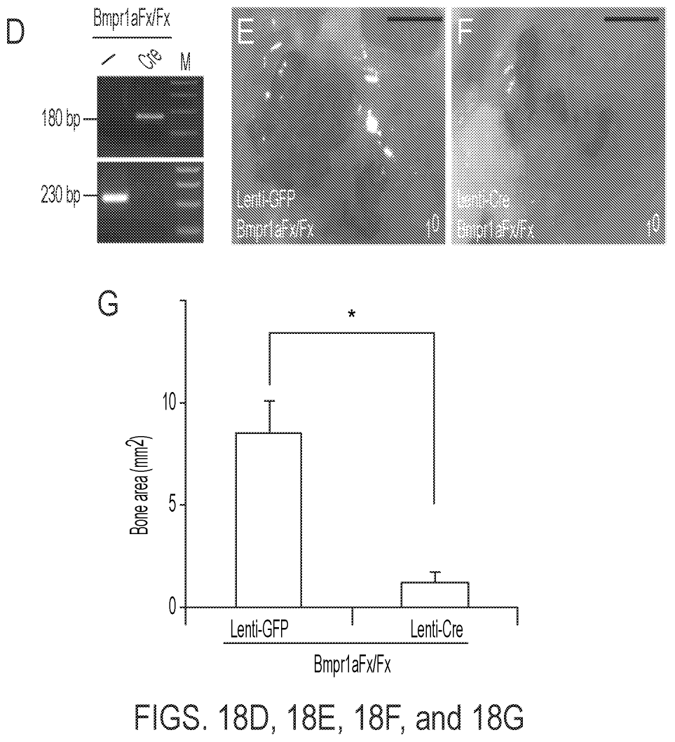

To exclude potential non-cell-autonomous effects on SuSCs that occur prior to isolation from the Bmpr1a-deficient mice using the Bmpr1a^{Ax2} model, the inventors used suture cells isolated from Bmpr1a^{Fx/Fx} mice, infected the cells in culture with lentivirus expressing GFP (control) or Cre, grew the cells until they formed spheres, and then used the 1⁰ spheres for kidney capsule transplantation. The efficiency of lentivirus-mediated expression that had minimal toxicity was determined with lentivirus expressing RFP: At a multiplicity of infection (MOI) of 1, the expression seemed optimal without notable changes in sphere size or number (FIG. 18A-C). Cre-dependent deletion of Bmpr1a in suture spheres was highly efficient (FIG. 18D) and drastically reduced the size of the generated bone tissue when transplanted into the kidney capsule (FIG. 18E-G; p-value <0.05, n=3, mean±SEM, student t-test).

These results with those from the Bmpr1a^{Ax2} model suggested that Bmpr1a has two key roles: Bmpr1a supports asymmetric division and clonal expansion of SuSCs (FIG. 6) and bone formation of SuSCs in a cell-autonomous manner (FIG. 6 and FIG. 18). Thus, the data indicated that Bmpr1a regulates not only SuSC self-renewal but also SuSC-mediated skeletogenesis.

### Example 10 Characterization of Human SuSCs

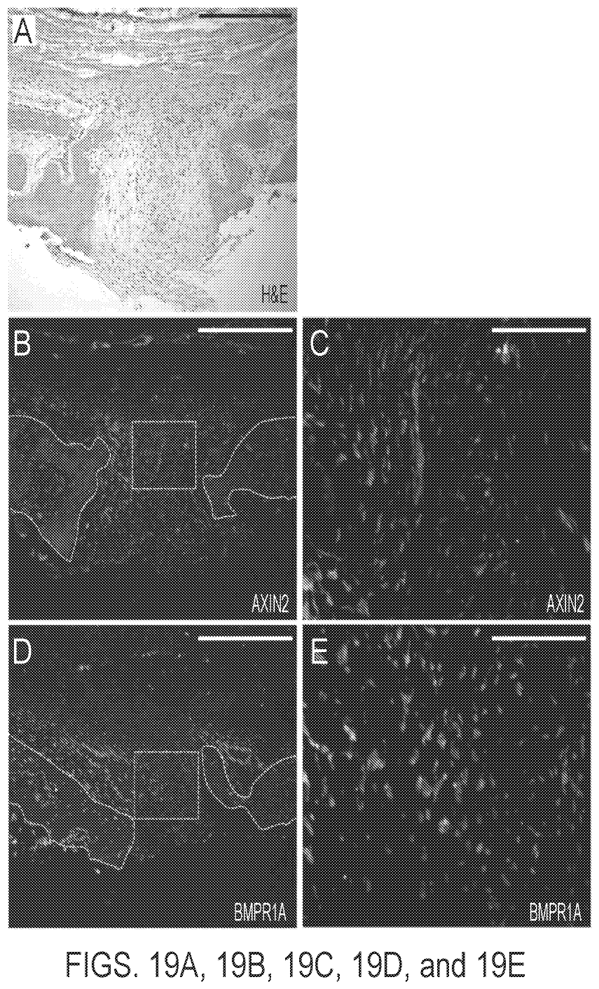

To test for the existence of human SuSCs and the ability to isolate them and maintain them in culture, the inventors obtained discarded tissue containing unfused sutures from craniosynostosis patients undergoing surgical operations. First, the inventors detected AXIN2-positive cells and BMPR1A-positive cells in the midline of human sutures by immunostaining of tissue sections (FIG. 7A-C and FIG. 19). The inventors determined that the isolated human suture cells grow into 1⁰ spheres when cultured in single-cell suspension with very low seeding density (FIG. 7d). the inventors obtained 2⁰ and 3⁰ spheres without notable decreases in number and size after serial re-plating (10⁴ cells for 1⁰-3⁰), indicating the presence of human SuSCs with self-renewing ability (FIG. 7E, F). Human suture spheres stained for AXIN2 and co-labeled with EdU revealed that, in spheres with an AXIN2+ cell, this cell was not actively proliferating (FIG. 7G-I). Some human spheres did not contain an AXIN2+ cell. These results indicated that human SuSCs are quiescent/slow-cycling cells and maintain their stemness through asymmetric division.

Finally, implantation of human cells into mouse kidney capsules revealed the formation of ectopic bones positive for von Kossa staining in whole mounts and sections (FIG. 7J-K) with an 80% success rate (n=5). The findings disclosed herein demonstrated successful isolation and culture of human SuSCs, a major hurdle to overcome for translational study.

### Example 11 Bone Formation from Mouse and Human Bmpr1a-Expressing Cells

The important role of Bmpr1a in stem cell regulation and the overlap in Bmpr1a and Axin2 positivity (FIG. 10E) prompted to test its use as a cell surface marker for SuSC isolation. Using a specific antibody and fluorescence-activated cell sorting (FACS), the inventors purified Bmpr1a^{High} from mouse (BMPR1A^{High} from human) and Bmpr1a/BMPR1A^{Low} cell populations from mouse/human sutures (FIG. 8A and FIG. 20). Mouse suture cells were from P10 C57/BL6 mice; human cells were from discarded calvarial tissues containing unfused suture of craniosynostosis patients. Successful bone formation was evident in the animal recipients with implantation of mouse Bmpr1a^{High} but not Bmpr1a^{Low} mouse suture cells (FIG. 8B-C). Immunostaining of Osterix identified osteoprogenitor cells surrounding the mineralized tissues generated by transplantation of mouse Bmpr1a^{High} (FIG. 8D-F) cells. The inventors achieved the same results with human BMPR1A^{High} suture cells (FIG. 8G-L). The results indicated that Bmpr1a/BMPR1A functions as a SuSC marker. Furthermore, these results confirmed that Bmpr1a not only functionally regulates stem cell sternness that is essential for suture patency and craniosynostosis but also that SuSCs are included in the Bmpr1a^{High} cell population in both mice and humans.

### Discussion

This application provides evidence that Bmpr1a is essential for SuSC regulation. Loss of Bmpr1a in Axin2-expressing cells impaired SuSC self-renewal, clonal expansion, and osteogenic abilities. Thus, Bmpr1a was required for maintaining these functions associated with stem cell sternness, implying a role for this receptor in repressing differentiation or in promoting asymmetric cell division. A suppressive effect of BMP signaling on early osteogenesis is supported by prior reports showing that neonatal disruption of Bmpr1a or its ablation in osteoprogenitor cells increases the osteoblast cell number (35-37). Loss of Bmpr1a reduced Smad phosphorylation and enhanced the activation of p38 and ERK, suggesting that the balance of canonical and noncanonical BMP signaling cascades is altered in SuSCs. Alternatively, the inactivation caused hyperactivation of signaling downstream of the other BMP type I receptors. It has been proposed that Bmpr1a regulates this balance through modulation of Tak1 activity (38). Because Tak1 was not activated in the Bmpr1a^{Ax2} mutant, the findings disclosed herein suggested a noncanonical pathway distinct from Tak1-mediated MAPK signaling is responsible for Bmpr1a-mediated SuSC sternness.

In the kidney capsule transplantation, only suture cells positive but not those negative for Axin2 can generate bones (5). This implies that skeletal stem cells included in the Axin2-positive cell population have bone-forming ability in the kidney capsule. Even though there are osteogenic precursors or osteoblast cells within the Axin2-negative cell population, these cells are unable to form ectopic bones (5). The requirement for Axin2-positive cells for ectopic bone generation may explain why direct engraftment and replacement of damaged tissue are difficult to achieve in most cell-based therapies. The number of Axin2-positive cells is too few in most therapies, and osteoblasts, despite being the bone-forming cells in vivo, are ineffective for bone formation upon transplantation. For therapeutic success, the survival, engraftment, and expansion of the transplanted cells seem highly critical factors. Only stem cells possess these properties, and the inventors found that those properties are preserved by Bmpr1a using both an in vivo ablation model and a culture deletion model. Further elucidation of the regulatory mechanism underlying cell survival and engraftment promises important insight into Bmpr1a-mediated bone regeneration, leading to a new strategy for stem cell-based therapy.

SuSC-specific ablation of Bmpr1a resulted in precocious differentiation and suture fusion. The findings disclosed herein revealed a new etiology for craniosynostosis—stem cell depletion. This pathogenetic mechanism is distinct from other known mechanisms: cell proliferation, differentiation, and apoptosis, any of which cause excessive intramembranous ossification (14). It is also different from the previous report in which suture fusion can be caused by stem cell fate switching: SuSCs undergo chondrogenesis instead of osteoblastogenesis, leading to craniosynostosis mediated by ectopic endochondral ossification (18). Stem cell depletion has previously been associated with ossification deficiency that may be related to patients with Cole-Carpenter syndrome that exhibit wide-open midline sutures containing intra-sutural bones (39). Intra-sutural bone, which is also known as Wormian bone, occurs frequently in disorders with reduced cranial ossification and has been associated with craniosynostosis (40). The stem cell depletion mechanism that the inventors identified herein can be used in synostosis patients without the enhanced ossification phenotype.

Although skeletal stem cells residing in the suture were identified, their role in craniosynostosis had not been investigated (5, 6). Axin2 and Gli1 have been used to identify skeletal stem cells in the calvarium (5, 6) but the deletion of Bmpr1a in Gli1+ cells does not induce craniosynostosis, despite resulting in enhanced osteoblast proliferation and differentiation (41). This discrepancy may be attributed to the presence of Axin2 in a more restricted cell population in the suture midline (5, 6). Also, Bmpr1a co-localizes with Axin2 but not Gli1 (41). The results disclosed herein showed that Axin2+ SuSCs undergo asymmetric division to maintain quiescence. The inventors believe that disruption of Bmpr1a-dependent regulation of SuSC quiescence is likely the trigger for craniosynostosis. The inventors propose that because SuSC stemness is maintained by Bmpr1a, its deletion leads to aberrant ossification initiated at the suture midline. Therefore, craniosynostosis arises from skeletal stem cell deficiency.

Preserving stemness in vitro is critical for engineering bone tissue. Although sphere-forming cells from bone marrow have been reported, there is a lack of evidence regarding their in vivo origin and osteogenic ability (42-44). Whether their stemness is preserved in vitro remains unknown. The inventors developed an ex vivo protocol to culture SuSCs for an extended period. The cultured SuSCs generated bone tissue upon implantation into an ectopic site. Furthermore, the SuSC culture provides an outstanding system for examination of skeletal stem cell characteristics, such as asymmetric division, cell fate determination, generation of skeletal progenitors, and skeletogenic differentiation. Thus, this system represents a tool for advancing stem cell-based therapy for bone regeneration and repair.

For application for humans, the inventors identified human BMPR1A-positive SuSCs capable of generating ectopic bone tissue. Because Axin2 is an intracellular protein, it is essential to identify a surface marker for stem cell purification. The BMP antagonist Gremlin1 labels skeletal stem cells that contribute to endochondral ossification; however, the functional importance of Gremlin1 remains unclear (8). The results disclosed herein demonstrated that Bmpr1a is not only a key regulator of SuSCs but also serves as a marker for their isolation. The inventors showed that BMPR1A, which is also known as CD292 (45), can be used to isolate human or mouse suture cells that possess skeletal stem cell properties for bone formation. The findings provide compelling evidence that BMPR1A positivity can be used for the purification of the human SuSC population.

Inclusion of SuSC niche cells is important for ectopic suture generation. The SuSC study disclosed herein promotes niche cell identification and isolation. The cells, compositions, and methods disclosed herein can be used for the prevention of suture re-synostosis in surgical patients or a preventive procedure for premature suture closure as an alternative to surgery for patients with craniosynostosis. The cells, compositions, and methods disclosed herein can also be used to elucidate the mechanism underlying SuSC regulation and SuSC-mediated regeneration. They can also be used for treating or preventing congenital deformity and for skeletal repair.

### REFERENCES

1. C. Mauffrey, B. T. Barlow, W. Smith, Management of segmental bone defects. J Am Acad Orthop Surg 23, 143-153 (2015).
2. P. Hernigou, A. Poignard, F. Beaujean, H. Rouard, Percutaneous autologous bone-marrow grafting for nonunions. Influence of the number and concentration of progenitor cells. J Bone Joint Surg Am 87, 1430-1437 (2005).
3. D. D. Lo, D. T. Montoro, M. Grova, J. S. Hyun, M. T. Chung, D. C. Wan, M. T. Longaker, Stem Cell-Based Bioengineering of Craniofacial Bone. 379-394 (2013).
4. N. J. Panetta, D. M. Gupta, M. T. Longaker, Bone regeneration and repair. Curr Stem Cell Res Ther 5, 122-128 (2010).
5. T. Maruyama, J. Jeong, T. J. Sheu, W. Hsu, Stem cells of the suture mesenchyme in craniofacial bone development, repair and regeneration. Nature communications 7, 10526 (2016).
6. H. Zhao, J. Feng, T. V. Ho, W. Grimes, M. Urata, Y. Chai, The suture provides a niche for mesenchymal stem cells of craniofacial bones. Nature cell biology 17, 386-396 (2015).
7. C. K. Chan, E. Y. Seo, J. Y. Chen, D. Lo, A. McArdle, R. Sinha, R. Tevlin, J. Seita, J. Vincent-Tompkins, T. Wearda, W. J. Lu, K. Senarath-Yapa, M. T. Chung, O. Marecic, M. Tran, K. S. Yan, R. Upton, G. G. Walmsley, A. S. Lee, D. Sahoo, C. J. Kuo, I. L. Weissman, M. T. Longaker, Identification and specification of the mouse skeletal stem cell. Cell 160, 285-298 (2015).
8. D. L. Worthley, M. Churchill, J. T. Compton, Y. Tailor, M. Rao, Y. Si, D. Levin, M. G. Schwartz, A. Uygur, Y. Hayakawa, S. Gross, B. W. Renz, W. Setlik, A. N. Martinez, X. Chen, S. Nizami, H. G. Lee, H. P. Kang, J. M. Caldwell, S. Asfaha, C. B. Westphalen, T. Graham, G. Jin, K. Nagar, H. Wang, M. A. Kheirbek, A. Kolhe, J. Carpenter, M. Glaire, A. Nair, S. Renders, N. Manieri, S. Muthupalani, J. G. Fox, M. Reichert, A. S. Giraud, R. F. Schwabe, J. P. Pradere, K. Walton, A. Prakash, D. Gumucio, A. K. Rustgi, T. S. Stappenbeck, R. A. Friedman, M. D. Gershon, P. Sims, T. Grikscheit, F. Y. Lee, G. Karsenty, S. Mukherjee, T. C. Wang, Gremlin 1 identifies a skeletal stem cell with bone, cartilage, and reticular stromal potential. Cell 160, 269-284 (2015).
9. P. T. Newton, L. Li, B. Zhou, C. Schweingruber, M. Hovorakova, M. Xie, X. Sun, L. Sandhow, A. V. Artemov, E. Ivashkin, S. Suter, V. Dyachuk, M. El Shahawy, A. Gritli-Linde, T. Bouderlique, J. Petersen, A. Mollbrink, J. Lundeberg, G. Enikolopov, H. Qian, K. Fried, M. Kasper, E. Hedlund, I. Adameyko, L. Savendahl, A. S. Chagin, A radical switch in clonality reveals a stem cell niche in the epiphyseal growth plate. Nature 567, 234-238 (2019).
10. K. Mizuhashi, W. Ono, Y. Matsushita, N. Sakagami, A. Takahashi, T. L. Saunders, T. Nagasawa, H. M. Kronenberg, N. Ono, Resting zone of the growth plate houses a unique class of skeletal stem cells. Nature 563, 254-258 (2018).
11. B. O. Zhou, R. Yue, M. M. Murphy, J. G. Peyer, S. J. Morrison, Leptin-receptor-expressing mesenchymal stromal cells represent the main source of bone formed by adult bone marrow. Cell Stem Cell 15, 154-168 (2014).
12. D. M. Ornitz, P. J. Marie, Fibroblast growth factor signaling in skeletal development and disease. Genes & development 29, 1463-1486 (2015).
13. C. K. Chan, C. C. Chen, C. A. Luppen, J. B. Kim, A. T. DeBoer, K. Wei, J. A. Helms, C. J. Kuo, D. L. Kraft, I. L. Weissman, Endochondral ossification is required for haematopoietic stem-cell niche formation. Nature 457, 490-494 (2009).
14. L. A. Opperman, Cranial sutures as intramembranous bone growth sites. Dev Dyn 219, 472-485 (2000).
15. A. O. Wilkie, G. M. Morriss-Kay, Genetics of craniofacial development and malformation. Nature reviews. Genetics 2, 458-468 (2001).
16. H. M. Yu, B. Jerchow, T. J. Sheu, B. Liu, F. Costantini, J. E. Puzas, W. Birchmeier, W. Hsu, The role of Axin2 in calvarial morphogenesis and craniosynostosis. Development (Cambridge, England) 132, 1995-2005 (2005).
17. E. Yilmaz, E. Mihci, B. Guzel Nur, O. M. Alper, A novel AXIN2 gene mutation in sagittal synostosis. Am J Med Genet A 176, 1976-1980 (2018).
18. T. Maruyama, A. J. Mirando, C. X. Deng, W. Hsu, The balance of WNT and FGF signaling influences mesenchymal stem cell fate during skeletal development. Sci Signal 3, ra40 (2010).
19. A. Kramer, J. Green, J. Pollard, Jr., S. Tugendreich, Causal analysis approaches in Ingenuity Pathway Analysis. Bioinformatics 30, 523-530 (2014).
20. X. Li, X. Cao, BMP signaling and skeletogenesis. Ann N Y Acad Sci 1068, 26-40 (2006).
21. J. Massague, TGF-beta signal transduction. Annu Rev Biochem 67, 753-791 (1998).
22. Y. Mishina, A. Suzuki, N. Ueno, R. R. Behringer, Bmpr encodes a type I bone morphogenetic protein receptor that is essential for gastrulation during mouse embryogenesis. Genes & development 9, 3027-3037 (1995).
23. Z. Gu, E. M. Reynolds, J. Song, H. Lei, A. Feijen, L. Yu, W. He, D. T. MacLaughlin, J. van den Eijnden-van Raaij, P. K. Donahoe, E. Li, The type I serine/threonine kinase receptor ActRIA (ALK2) is required for gastrulation of the mouse embryo. Development (Cambridge, England) 126, 2551-2561 (1999).
24. Y. Mishina, R. Crombie, A. Bradley, R. R. Behringer, Multiple roles for activin-like kinase-2 signaling during mouse embryogenesis. Developmental biology 213, 314-326 (1999).
25. S. E. Yi, A. Daluiski, R. Pederson, V. Rosen, K. M. Lyons, The type I BMP receptor BMPRIB is required for chondrogenesis in the mouse limb. Development (Cambridge, England) 127, 621-630 (2000).
26. H. Williams, Lumps, bumps and funny shaped heads. Arch Dis Child Educ Pract Ed 93, 120-128 (2008).
27. S. Nakata, Relationship between the development and growth of cranial bones and masticatory muscles in postnatal mice. J Dent Res 60, 1440-1450 (1981).
28. B. L. Hogan, Bone morphogenetic proteins: multifunctional regulators of vertebrate development. Genes Dev 10, 1580-1594 (1996).
29. M. S. Rahman, N. Akhtar, H. M. Jamil, R. S. Banik, S. M. Asaduzzaman, TGF-beta/BMP signaling and other molecular events: regulation of osteoblastogenesis and bone formation. Bone Res 3, 15005 (2015).
30. M. B. Greenblatt, J. H. Shim, W. Zou, D. Sitara, M. Schweitzer, D. Hu, S. Lotinun, Y. Sano, R. Baron, J. M. Park, S. Arthur, M. Xie, M. D. Schneider, B. Zhai, S. Gygi, R. Davis, L. H. Glimcher, The p38 MAPK pathway is essential for skeletogenesis and bone homeostasis in mice. J Clin Invest 120, 2457-2473 (2010).
31. Y. Wang, M. Sun, V. L. Uhlhorn, X. Zhou, I. Peter, N. Martinez-Abadias, C. A. Hill, C. J. Percival, J. T. Richtsmeier, D. L. Huso, E. W. Jabs, Activation of p38 MAPK pathway in the skull abnormalities of Apert syndrome Fgfr2(+P253R) mice. BMC Dev Biol 10, 22 (2010).
32. V. Shukla, X. Coumoul, R. H. Wang, H. S. Kim, C. X. Deng, RNA interference and inhibition of MEK-ERK signaling prevent abnormal skeletal phenotypes in a mouse model of craniosynostosis. Nature genetics 39, 1145-1150 (2007).
33. Y. Hu, G. K. Smyth, ELDA: extreme limiting dilution analysis for comparing depleted and enriched populations in stem cell and other assays. Journal of immunological methods 347, 70-78 (2009).
34. E. Pastrana, V. Silva-Vargas, F. Doetsch, Eyes wide open: a critical review of sphere-formation as an assay for stem cells. Cell Stem Cell 8, 486-498 (2011).
35. J. Lim, Y. Shi, C. M. Kamer, S. Y. Lee, W. C. Lee, G. He, F. Long, Dual function of Bmpr1a signaling in restricting preosteoblast proliferation and stimulating osteoblast activity in mouse. Development (Cambridge, England) 143, 339-347 (2016).
36. J. Zhang, C. Niu, L. Ye, H. Huang, X. He, W. G. Tong, J. Ross, J. Haug, T. Johnson, J. Q. Feng, S. Harris, L. M. Wiedemann, Y. Mishina, L. Li, Identification of the haematopoietic stem cell niche and control of the niche size. Nature 425, 836-841 (2003).
37. N. Kamiya, L. Ye, T. Kobayashi, Y. Mochida, M. Yamauchi, H. M. Kronenberg, J. Q. Feng, Y. Mishina, BMP signaling negatively regulates bone mass through sclerostin by inhibiting the canonical Wnt pathway. Development (Cambridge, England) 135, 3801-3811 (2008).
38. H. Y. Kua, H. Liu, W. F. Leong, L. Li, D. Jia, G. Ma, Y. Hu, X. Wang, J. F. Chau, Y. G. Chen, Y. Mishina, S. Boast, J. Yeh, L. Xia, G. Q. Chen, L. He, S. P. Goff, B. Li, c-Abl promotes osteoblast expansion by differentially regulating canonical and non-canonical BMP pathways and p16INK4a expression. Nature cell biology 14, 727-737 (2012).
39. D. J. Amor, R. Savarirayan, A. S. Schneider, A. Bankier, New case of Cole-Carpenter syndrome. American journal of medical genetics 92, 273-277 (2000).
40. P. A. Sanchez-Lara, J. M. Graham, Jr., A. V. Hing, J. Lee, M. Cunningham, The morphogenesis of wormian bones: a study of craniosynostosis and purposeful cranial deformation. Am J Med Genet A 143A, 3243-3251 (2007).
41. Y. Guo, Y. Yuan, L. Wu, T. V. Ho, J. Jing, H. Sugii, J. Li, X. Han, J. Feng, C. Guo, Y. Chai, BMP-IHH-mediated interplay between mesenchymal stem cells and osteoclasts supports calvarial bone homeostasis and repair. Bone Res 6, 30 (2018).
42. J. Isern, B. Martin-Antonio, R. Ghazanfari, A. M. Martin, J. A. Lopez, R. del Toro, A. Sanchez-Aguilera, L. Arranz, D. Martin-Perez, M. Suarez-Lledo, P. Marin, M. Van Pel, W. E. Fibbe, J. Vazquez, S. Scheding, A. Urbano-Ispizua, S. Mendez-Ferrer, Self-renewing human bone marrow mesenspheres promote hematopoietic stem cell expansion. Cell reports 3, 1714-1724 (2013).
43. M. Shiota, T. Heike, M. Haruyama, S. Baba, A. Tsuchiya, H. Fujino, H. Kobayashi, T. Kato, K. Umeda, M. Yoshimoto, T. Nakahata, Isolation and characterization of bone marrow-derived mesenchymal progenitor cells with myogenic and neuronal properties. Experimental cell research 313, 1008-1023 (2007).
44. S. Debnath, A. R. Yallowitz, J. McCormick, S. Lalani, T. Zhang, R. Xu, N. Li, Y. Liu, Y. S. Yang, M. Eiseman, J. H. Shim, M. Hameed, J. H. Healey, M. P. Bostrom, D. A. Landau, M. B. Greenblatt, Discovery of a periosteal stem cell mediating intramembranous bone formation. Nature 562, 133-139 (2018).
45. P. Engel, L. Boumsell, R. Balderas, A. Bensussan, V. Gattei, V. Horejsi, B. Q. Jin, F. Malavasi, F. Mortari, R. Schwartz-Albiez, H. Stockinger, M. C. van Zelm, H. Zola, G. Clark, CD Nomenclature 2015: Human Leukocyte Differentiation Antigen Workshops as a Driving Force in Immunology. J Immunol 195, 4555-4563 (2015).
46. H. M. Yu, B. Liu, F. Costantini, W. Hsu, Impaired neural development caused by inducible expression of Axin in transgenic mice. Mechanisms of development 124, 146-156 (2007).
47. H. M. Yu, B. Liu, S. Y. Chiu, F. Costantini, W. Hsu, Development of a unique system for spatiotemporal and lineage-specific gene expression in mice. Proceedings of the National Academy of Sciences of the United States of America 102, 8615-8620 (2005).
48. Y. Mishina, M. C. Hanks, S. Miura, M. D. Tallquist, R. R. Behringer, Generation of Bmpr/Alk3 conditional knockout mice. Genesis 32, 69-72 (2002).
49. M. Dudas, S. Sridurongrit, A. Nagy, K. Okazaki, V. Kaartinen, Craniofacial defects in mice lacking BMP type I receptor Alk2 in neural crest cells. Mechanisms of development 121, 173-182 (2004).
50. L. Madisen, T. A. Zwingman, S. M. Sunkin, S. W. Oh, H. A. Zariwala, H. Gu, L. L. Ng, R. D. Palmiter, M. J. Hawrylycz, A. R. Jones, E. S. Lein, H. Zeng, A robust and high-throughput Cre reporting and characterization system for the whole mouse brain. Nature neuroscience 13, 133-140 (2010).
51. T. Tumbar, G. Guasch, V. Greco, C. Blanpain, W. E. Lowry, M. Rendl, E. Fuchs, Defining the epithelial stem cell niche in skin. Science (New York, N.Y. 303, 359-363 (2004).
52. W. Hsu, A. J. Mirando, H. M. Yu, Manipulating gene activity in Wnt1-expressing precursors of neural epithelial and neural crest cells. Dev Dyn 239, 338-345 (2010).
53. E. O. Maruyama, H. M. Yu, M. Jiang, J. Fu, W. Hsu, Gpr177 deficiency impairs mammary development and prohibits Wnt-induced tumorigenesis. PLoS ONE 8, e56644 (2013).
54. H. J. Snippert, L. G. van der Flier, T. Sato, J. H. van Es, M. van den Born, C. Kroon-Veenboer, N. Barker, A. M. Klein, J. van Rheenen, B. D. Simons, H. Clevers, Intestinal crypt homeostasis results from neutral competition between symmetrically dividing Lgr5 stem cells. Cell 143, 134-144 (2010).
55. J. Fu, M. Jiang, A. J. Mirando, H. M. Yu, W. Hsu, Reciprocal regulation of Wnt and Gpr177/mouse Wntless is required for embryonic axis formation. Proceedings of the National Academy of Sciences of the United States of America 106, 18598-18603 (2009).
56. A. J. Mirando, T. Maruyama, J. Fu, H. M. Yu, W. Hsu, Beta-catenin/cyclin D1 mediated development of suture mesenchyme in calvarial morphogenesis. BMC Dev Biol 10, 116 (2010).
57. Y. Hu, G. K. Smyth, ELDA: extreme limiting dilution analysis for comparing depleted and enriched populations in stem cell and other assays. J Immunol Methods 347, 70-78 (2009).
58. T. Maruyama, M. Jiang, W. Hsu, Gpr177, a novel locus for bone mineral density and osteoporosis, regulates osteogenesis and chondrogenesis in skeletal development. J Bone Miner Res 28, 1150-1159 (2013).
59. S. Y. Chiu, N. Asai, F. Costantini, W. Hsu, SUMO-Specific Protease 2 Is Essential for Modulating p53-Mdm2 in Development of Trophoblast Stem Cell Niches and Lineages. PLoS biology 6, e310 (2008).
60. J. Fu, W. Hsu, Epidermal Wnt controls hair follicle induction by orchestrating dynamic signaling crosstalk between the epidermis and dermis. J Invest Dermatol 133, 890-898 (2013).
61. H. K. Russell, Jr., A modification of Movat's pentachrome stain. Archives of pathology 94, 187-191 (1972).
62. J. Fu, H. M. Yu, S. Y. Chiu, A. J. Mirando, E. O. Maruyama, J. G. Cheng, W. Hsu, Disruption of SUMO-Specific Protease 2 Induces Mitochondria Mediated Neurodegeneration. PLoS genetics 10, e1004579 (2014).
63. T. Maruyama, M. Jiang, A. Abbott, H. I. Yu, Q. Huang, M. Chrzanowska-Wodnicka, E. I. Chen, W. Hsu, Raplb is an effector of Axin2 regulating crosstalk of signaling pathways during skeletal development. J Bone Miner Res, (2017).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A method for identifying or isolating or enriching a skeletal stem cell, comprising
obtaining a start cell population;
identifying from the start cell population a cell expressing a BMPR1A polypeptide (BMPR1A⁺ cell), or expressing an mRNA encoding the BMPR1A polypeptide.

2. The method of claim 1, further comprising isolating the identified BMPR1A⁺ cell.

3. The method of claim 2, further comprising collecting a plurality of BMPR1A⁺ cells to obtain an enriched skeletal stem cell population.

4. The method of claim 1, wherein the cells express one or more markers selected from the group consisting of Axin2, BMP2, BMP3, BMP4, BMP6, BMP7, BMP8b, and BMP15.

5. The method of claim 2, wherein the BMPR1A⁺ cell or cells are isolated using a protein, a polypeptide, or a composition that specifically binds to BMPR1A.

6. The method of claim 5, wherein the protein comprises an anti-BMPR1A antibody or an antigen-binding fragment thereof.

7. The method of claim 5, wherein the protein, peptide, or composition comprises a ligand of BMPR1A or a BMPR1A-binding fragment thereof.

8. The method of claim 7, wherein the ligand is selected from the group consisting of BMP2, BMP4, BMP6, BMP7, and GDF6.

9. The method of claim 1, wherein the BMPR1A⁺ cell or cells are identified or isolated or enriched by fluorescence activated cell sorting.

10. The method of claim 1, wherein the start cell population is from a tissue of a subject.

11. The method of claim 10, wherein the subject is a mammal.

12. The method of claim 11, wherein the mammal is a human.

13. The method of claim 1, wherein the start cell population comprises one or more selected from the group consisting of bone marrow, cord blood cells, embryonic stem cells or progenies thereof, mesenchymal stem cells or progenies thereof, and induced pluripotent stem cells (iPSCs) or progenies thereof.

14. The method of claim 13, wherein the mesenchymal stem cells are suture mesenchymal stem cells.

15. A method for maintaining suture stem cells or skeletal stem cells in vitro, comprising
(i) providing a population of suture stem cells or skeletal stem cells;
(ii) seeding the cells in a maintaining medium on an ultra-low attachment surface; and
(iii) culturing the cells or the progenies thereof for a period of time to form one or more spheres.

16-24. (canceled)

25. A sphere of cells comprising one or more BMPR1A⁺ cells.

26-30. (canceled)

31. A composition comprising (i) a carrier and (ii) one or more BMPR1A⁺ cells, or one or more spheres of claim 25, or cells derived from the one or more spheres.

32-33. (canceled)

34. A bone regeneration product or formulation comprising (i) the composition of claim 31 and (ii) a scaffold.

35. A method for generating or regenerating cartilage or bone in a subject, comprising administering to a subject in need thereof an effective amount of the composition of claim 31 at a site where regeneration of bone or cartilage is desired.

36. A method of generating skeletal, stromal, or cartilaginous tissue, comprising
(i) providing one or more BMPR1A⁺ cells, or one or more spheres of claim 25, or cells derived from the one or more spheres, and
(ii) inducing differentiation of the bmpr1a⁺ cells, or of the cells from the one or more spheres.
